(19) United States
(12) Patent Application Publication
(10) Pub. No.: US 2008/0103130 A1
(43) Pub. Date: May 1, 2008

(54) BENZAZEPIN-2(1H)-ONE DERIVATIVES

(75) Inventors: Jessica Boyle, Sandwich (GB); Ashley Edward Fenwick, Sandwich (GB); David Morris Gethin, Sandwich (GB); Catherine Frances McCusker, Sandwich (GB)

Correspondence Address: PHARMACIA & UPJOHN, 7000 Portage Road, KZO-300-104, KALAMAZOO, MI, 49001, US

(73) Assignee: PFIZER LIMITED, Sandwich (GB)

(21) Appl. No.: 11/877,861

(22) Filed: Oct. 24, 2007

Related U.S. Application Data
(60) Provisional application No. 60/862,868, filed on Oct. 25, 2006.

(51) Int. Cl. A61K 31/55; A61P 43/00; C07D 487/02

(52) U.S. Cl. 514/214.02; 540/579

(57) ABSTRACT

Compounds of formula (I) 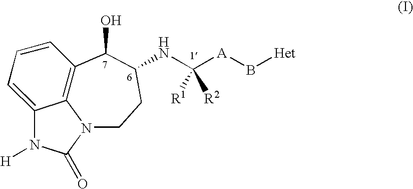 and pharmaceutically acceptable salts thereof are agonists at the beta-2 adrenoceptor. They are useful as feed additives for livestock animals.

### CROSS-REFERENCE TO RELATED APPLICATIONS

This application claims priority to U.S. Provisional Application No. 60/862,868 filed Oct. 25, 2006.

The present invention relates to a series of 6-amino-7-hydroxy-4,5,6,7-tetrahydro-imidazo[4,5,1-jk][1]benzazepin-2(1H)-ones. More particularly it relates to a series of 6-(heteroarylalkyl)amino-7-hydroxy-4,5,6,7-tetrahydroimidazo[4,5,1-jk][1]benzazepin-2(1H)-ones. The compounds act as agonists at the beta-2 adrenoceptor and are useful as anabolic agents for livestock animals.

### BACKGROUND

The primary focus in livestock production remains efficiency via optimising the conversion of feed into lean meat. Feed constitutes a high proportion of the total economic investment in the final stages of livestock production, and hence there is a continued demand for agents which enhance feed conversion ratio (FCR). The most effective way of improving FCR is via metabolic manipulation to enhance the animals' potential to deposit muscle protein, which also provides obvious benefits in yield grade and carcass composition.

One approach to achieving higher quality meat and improving the meat yield is to administer agents that are agonists at the beta-2 adrenoceptor. Examples of agents registered for such use in livestock animals are Zilmax™ (zilpaterol) and Optaflexx™ (ractopamine). Zilpaterol is (±)-trans-6-(isopropylamino)-7-hydroxy-4,5,6,7-tetrahydro-imidazo[4,5,1-jk][1]benzazepin-2(1H)-one. Zilpaterol and similar analogues were first disclosed in FR2534257 and subsequently their use as animal feed additives was discussed in FR2608046 and EP272976. Ractopamine is (±)-4-(3-{[2-hydroxy-2-(4-hydroxyphenyl)ethyl]amino}butyl)phenol and was first disclosed by van Dijk and Moed (Recl. Trav. Chim. Pays Bas, 1973, 92, 1281-12799). Its use as a feed additive was described in GB2133986. Both zilpaterol and ractopamine are administered during the latter stages of a production animal's life and cause an activation of a biological cascade mechanism, starting with interaction at the beta2 adrenoceptor, which promotes and enhances lean muscle growth. A series of aryloxypropanolamines for improving livestock production have been recently disclosed in U.S. Pat. No. 6,841,563.

There is a continuing need for alternative beta-2 adrenoceptor agonists for use as agents to improve meat production in livestock animals, and particularly for agonists with improved properties. For reasons of economy, the agent should preferably provide the desired improvement in meat production at a low dose. It must also not produce any undesired effects in the target animal. Finally, the meat produced by the animal must be safe for human consumption, which implies that the residual levels of the agent in the meat must be minimised. The ideal agent will therefore have a high affinity for, and be a fully efficacious agonist at, the beta-2 adrenoceptor of the target animal species. It will have a high degree of selectivity for this receptor, and it will be rapidly cleared from the animal in order to minimise the presence of residues in the meat without requiring an extended withdrawal period. A zero-day withdrawal period provides the maximum economic benefit to the farmer. Thus it is an aim of this invention to provide compounds which have a high affinity, selectivity, agonist efficacy and/or potency at the beta-2 adrenoceptor of relevant livestock animals, and/or that are rapidly metabolically cleared from the animal.

### SUMMARY OF THE INVENTION

In a first aspect, the present invention provides a compound of formula (I) 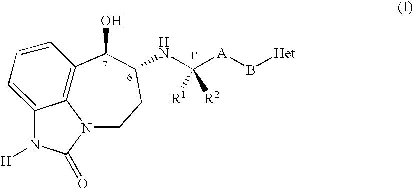
or a pharmaceutically acceptable salt thereof, wherein:
A is CH₂, CH(C₁-C₃ alkyl) or C(C₁-C₃ alkyl)₂; and
B is a covalent bond, —CR^{A}R^{B}—, —CR^{A}R^{B}—CR^{C}R^{D}—, —CR^{A}R^{B}—CR^{C}R^{D}—CR^{E}R^{F}—, —CR^{A}R^{B}—O—, —O—CR^{A}R^{B}, —O—CR^{A}R^{B}—CR^{C}R^{D}, —CR^{A}R^{B}—O—CR^{C}R^{D}, or —CR^{A}R^{B}—CR^{C}R^{D}—O—;
or -A-B— is —CR^{A}═CR^{B}—;
R^{A}, R^{B}, R^{C}, R^{D}, R^{E} and R^{F} are each independently H or C₁-C₃ alkyl;
R¹ and R² are each independently H or C₁-C₃ alkyl, or R¹ and R² together with the carbon atom to which they are attached form a 3- to 6-membered saturated carbocyclic ring; and
Het is a 5- or 6-membered monocyclic or 9- or 10-membered bicyclic heteroaryl group which may optionally be substituted with up to 3 groups independently selected from halo, —CN, C₁-C₄ alkyl, —CH₂Ph, —OH, —O—(C₁-C₄ alkyl), —O—CH₂—(C₃-C₆)cycloalkyl, —O—CH₂Ph, —NH₂, —NH(C₁-C₄ alkyl), —N(C₁-C₄ alkyl)₂, —CONH₂, —CONH(C₁-C₄ alkyl), —CON(C₁-C₄ alkyl)₂, —CO₂H and —CO₂(C₁-C₄ alkyl).

In a further aspect, the present invention provides a feed additive for a livestock animal comprising a compound of formula (I) or a pharmaceutically acceptable salt thereof.

In a yet further aspect, the present invention provides a method of improving meat yield or meat quality in a livestock animal comprising administering to said livestock animal an effective amount of a compound of formula (I) or a pharmaceutically acceptable salt thereof.

In a yet further aspect, the present invention provides the use of a compound of formula (I) or a pharmaceutically acceptable salt thereof as a medicament.

In a yet further aspect, the present invention provides a pharmaceutical composition comprising a compound of formula (I) or a pharmaceutically acceptable salt thereof.

### DETAILED DESCRIPTION OF THE INVENTION

For the purposes of the present document, the following definitions apply.

“Alkyl” means a saturated monovalent hydrocarbon radical CₙH₂ₙ₊₁ which may be linear or branched. C₁-C₄ alkyl includes methyl, ethyl, n-propyl, isopropyl (1-methylethyl), n-butyl, sec-butyl (1-methylpropyl), isobutyl (2-methylpropyl) and tert-butyl (1,1-dimethylethyl).

“Cycloalkyl” means a saturated monovalent monocyclic or bridged or fused polycyclic hydrocarbon radical. C₃-C₅ cycloalkyl includes cyclopropyl, cyclobutyl and cyclopentyl.

“Halo” includes fluoro, chloro, bromo and iodo.

Haloalkyl means an alkyl group as defined above wherein one or more hydrogen atoms is replaced by a halogen atom selected from fluorine, chlorine, bromine and iodine. When the group contains more than one halogen atom then these atoms may be the same or different. Haloalkyl includes perhaloalkyl, i.e. an alkyl group wherein all the hydrogen atoms are replaced by halogen atoms. C₁-C₄ haloalkyl groups include fluoromethyl, difluoromethyl, trifluoromethyl, chlorodifluoromethyl, 2-bromoethyl, 2,2,2-trifluoroethyl, 3-iodopropyl, and 2,2,2-trichloro-1,1-dimethylethyl.

“Heteroaryl” means a monovalent monocyclic or fused bicyclic aromatic radical wherein at least one of the ring atoms is a heteroatom selected from nitrogen, oxygen and sulphur, and the remaining ring atoms are all carbon. The group may be attached through a carbon atom or, where chemically feasible, a nitrogen atom. In heteroaryl ring systems that include a carbonyl group (>C═O), the carbonyl oxygen is considered to be a part of the ring rather than a substituent on the ring. However, the oxygen is not included when counting the number of heteroatoms in the ring. For example, 2(1H)-pyridinone is considered to be an unsubstituted heteroaryl system with one ring heteroatom.

Monocyclic heteroaryl groups generally have no more than one oxygen or sulphur atom. Fused bicyclic heteroaryl groups may have one such atom in each ring, provided that the oxygen or sulphur atom is not shared by the two rings.

Bicyclic heteroaryl groups include bicyclic systems wherein only one of the rings incorporates a heteroatom.

When a heteroaryl group includes a nitrogen atom that has a hydrogen atom attached (i.e. a —NH— moiety) and the group is optionally substituted, then substitution at this nitrogen is permitted. This nitrogen is also available as a point of attachment.

5-Membered monocyclic heteroaryl groups include pyrrolyl (including 1-pyrrolyl, 2-pyrrolyl and 3-pyrrolyl), furyl (including 2-furyl and 3-furyl), thienyl (including 2-thienyl and 3-thienyl), pyrazolyl, imidazolyl (including 1-imidazolyl, 2-imidazolyl and 4-imidazolyl), oxazolyl, isoxazolyl, thiazolyl, isothiazolyl, 1,2,3-triazolyl, 1,2,4-triazolyl, oxadiazolyl and thiadiazolyl.

6-Membered monocyclic heteroaryl groups include pyridyl (including 2-pyridyl, 3-pyridyl and 4-pyridyl), 2(1H)-pyridinonyl (including 2(1H)-pyridinon-1-yl, 2(1H)-pyridinon-3-yl, 2(1H)-pyridinon-4-yl, 2(1H)-pyridinon-5-yl and 2(1H)-pyridinon-6-yl), 4(1H)-pyridinonyl (including 4(1H)-pyridinon-1-yl, 4(1H)-pyridinon-2-yl and 4(1H)-pyridinon-3-yl), pyran-2-onyl, pyran-4-onyl, pyridazinyl, pyrimidinyl and pyrazinyl.

9-Membered fused bicyclic heteroaryl groups include indolyl (including 1-indolyl, 2-indolyl, 3-indolyl, 4-indolyl, 5-indolyl, 6-indolyl and 7-indolyl), isoindolyl, benzofuryl, isobenzofuryl, benzothienyl, isobenzothienyl, benzoxazolyl, benzisoxazolyl, benzothiazolyl, benzisothiazolyl, indazolyl, benzimidazolyl, benzotriazolyl, indolizinyl, 1H-[1]pyrindinyl, 2H-[2]pyrindinyl, pyrrolo[2,3-b]pyridinyl, pyrrolo[3,2-b]pyridinyl, pyrrolo[2,3-c]pyridinyl, pyrrolo[3,2-c]pyridinyl, pyrrolo[3,4-b]pyridinyl, pyrrolo[3,4-c]pyridinyl, imidazo[1,2-a]pyridinyl, imidazo[1,5-a]pyridinyl, pyrazolo[1,5-a]pyridinyl, furo[2,3-b]pyridinyl, furo[3,2-b]pyridinyl, furo[2,3-c]pyridinyl, furo[3,2-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, imidazo[4,5-b]pyridinyl, imidazo[4,5-c]pyridinyl, purinyl and the like.

10-Membered fused bicyclic heteroaryl groups include quinolinyl (including 2-quinolinyl, 3-quinolinyl, 4-quinolinyl, 5-quinolinyl, 6-quinolinyl, 7-quinolinyl and 8-quinolinyl), quinolin-2-onyl, quinolin-4-onyl, isoquinolinyl, isoquinolin-1-onyl, isoquinolin-3-onyl, chromen-2-one, chromen-4-one, isochromen-1-one, isochromen-4-one, cinnolinyl, phthalazinyl, quinazolinyl, quinoxalinyl, [1,5]-naphthyridinyl, [1,6]-naphthyridinyl, [1,7]-naphthyridinyl, [1,8]-naphthyridinyl and the like.

The compounds of formula (I) have two asymmetric carbon atoms (chiral centres), labelled 6 and 7 in the structural formula. When R¹ and R² are different then the atom labelled 1′ is a third asymmetric carbon. Certain embodiments of the groups A and B may include additional chiral centres. Unless otherwise indicated, formula (I) depicts the relative stereochemistry at the three centres C-1′, C-6 and C-7. It is not intended that the representation of formula (I) should be taken as implying the absolute stereochemistry at these centres. Accordingly, the present invention includes individual enantiomers of the compounds of formula (I) and mixtures thereof, including racemates. Where there is an additional chiral centre then the invention includes diastereomeric mixtures as well as individual stereoisomers.

The compounds of formula (I) wherein -A-B— is —CR^{A}═CR^{B}— may exist as geometric isomers. Unless otherwise indicated, no particular geometry is implied by this notation. Accordingly, the present invention encompasses such compounds in the cis (Z-) or trans (E-) configuration, as well as mixtures of these geometric isomers.

Certain compounds of formula (I) may exist in more than one tautomeric form. The present invention encompasses all such tautomers, as well as mixtures thereof.

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

The compounds of formula (I) are able to form addition salts with acids. Certain compounds of formula (I) which have an acidic functional group are able to form salts with suitable bases. Such salts are included within the scope of the present invention to the extent that they are acceptable for veterinary or pharmaceutical use.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent.

The compounds of formula (I) and their salts may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of formula (I) and their salts may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Pharmaceutically acceptable solvates in accordance with the invention include those wherein the solvent of crystallization may be isotopically substituted, e.g. D₂O, d₆-acetone, d₆-DMSO.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of formula (I) and their salts may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The present invention also includes so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985).

Examples of prodrugs in accordance with the invention include
(i) derivatives of the C-7 hydroxyl function such as esters and acyloxymethyl ethers, wherein the hydrogen of the hydroxyl group is replaced by an acyl group such as (C₁-C₆ alkyl)CO— or (optionally substituted aryl)CO—, or by an acyloxymethyl group such as (C₁-C₆ alkyl)CO₂CH₂—; and
(ii) derivatives of the C-6 secondary amine function such as amides and carbamates, wherein the hydrogen of the amine group is replaced by an acyl group such as (C₁-C₆ alkyl)CO— or by an alkyloxycarbonyl group such as (C₁-C₆ alkyl)OCO—.

Certain of the options for the substituents on Het may also be amenable to the formation of prodrugs.

In a further aspect, the present invention provides processes for the preparation of a compound of formula (I), or a pharmaceutically, veterinarily or agriculturally acceptable salt thereof, or a pharmaceutically, veterinarily or agriculturally acceptable solvate (including hydrate) of either entity, as illustrated below.

It will be apparent to those skilled in the art that sensitive functional groups may need to be protected and deprotected during synthesis of a compound of the invention. This may be achieved by conventional methods, for example as described in “Protective Groups in Organic Synthesis” by TW Greene and PGM Wuts, John Wiley & Sons Inc (1999), and references therein.

The following processes are illustrative of the general synthetic procedures which may be adopted in order to obtain the compounds of the invention.

When the heteroaryl substituent contains one or more reactive functional groups then additional protection may be provided, according to standard procedures, during the synthesis of compounds of formula (I). In the processes described below, for all synthetic precursors used in the synthesis of compounds of formula (I), the definitions of Het are intended to optionally include suitably protected variants. Some suitable protecting groups for these functionalities are described in the references listed below and the use of these protecting groups where needed is specifically intended to fall within the scope of the processes described in the present invention for producing compounds of formula (I) and its precursors. When suitable protecting groups are used, then these will need to be removed to yield compounds of formula (I). Deprotection can be effected according to standard literature procedures including those described in the references listed below.

1. Preparation of Compounds of Formula (I)

1.1. Reductive Amination

Compounds of formula (I) wherein R¹, R²═H, C₁-C₃ alkyl, or C₁-C₃ alkyl, H may be synthesised by the reductive amination of the ketones of formula (II), wherein Het, A and B are as defined for formula (I), using the amino-alcohol of formula (III), as illustrated in Scheme A: 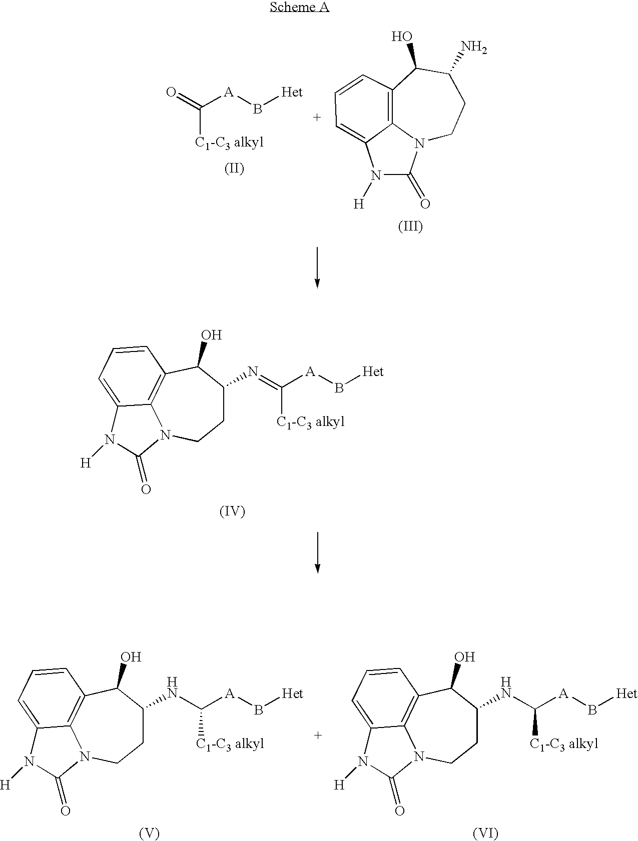
wherein the wedge and dashed bonds indicate the relative stereochemistry of the 6-amino and 7-hydroxy substituents. The skilled person will appreciate that the individual enantiomers or the racemate of formula (III) may be used for the reductive amination reaction.

A variety of reaction conditions may be used. In general, reaction of the amino-alcohol (III) with the ketones of formula (II) yields an imine, (IV), which may be reduced in situ to give compounds of formula (I). Imine formation is achieved by standard methods, for example, by reaction of the amino-alcohol (III) with the ketones (II) in an alcoholic solvent, preferably methanol, in the presence of a base, such as triethylamine or potassium hydroxide. Reaction conditions may vary from room temperature to 50° C. for periods ranging from 10 minutes to 60 hours, optionally under nitrogen and optionally heating in a microwave. Compounds of formula (I) may then be prepared by in situ imine reduction, typically using sodium borohydride or sodium cyanoborohydride, at temperatures ranging from 0° C. to 60° C. for 1-60 hours, typically overnight. The imine reduction proceeds with a range of diastereoselectivities, though no predictive trend has yet been observed.

Similarly, compounds of formula (I) wherein R¹, R²═H, H may be prepared by reductive amination with aldehydes of formula (VII) wherein Het, A and B are as defined for formula (I).

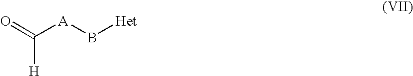

Compounds of formula (I) wherein A-B is CH═CH may be prepared using similar conditions to those described above by reductive amination of the amino-alcohol (III) with the α,β-unsaturated enones of formula (VIII) wherein Het is as defined for formula (I), as illustrated in Scheme B.

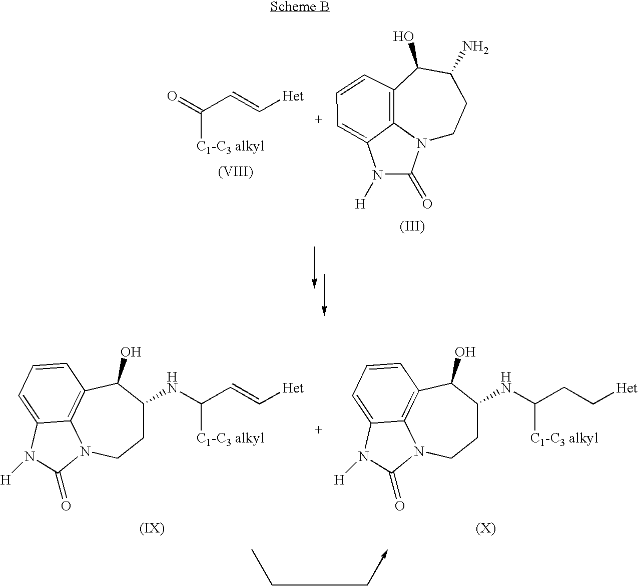

Using excess borohydride reducing agent will also reduce the double bond, so using enones of formula (VIII) may yield compounds of formula (I) wherein A-B is CH₂—CH₂ or A-B is CH═CH, i.e. compounds of formula (X) or compounds of formula (IX).

Compounds of formula (I) wherein A-B is CH₂—CH₂ may also be prepared from compounds of formula (I) wherein A-B is CH═CH using standard reducing agents, such as hydrogen in the presence of a metal catalyst such as Wilkinson's catalyst, palladium on carbon or platinum oxide in a protic solvent, for example methanol, or those described in “Handbook of Reagents for Organic Synthesis—Oxidising and Reducing Agents” edited by S. D. Burke and R. L. Danheiser.
1.2 Alkylation

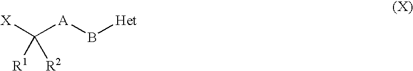

Compounds of formula (I) may also be prepared by reaction of the amino-alcohol of formula (III) with an alkylating agent of formula (X) where X may be any leaving group, typically 1, Br, Cl, OTs, OTf, O-mesylate, or O-trichloromethylsulphonate, in a suitable solvent, e.g. acetone, dichloromethane, acetonitrile, dimethylformamide or N-methylpyrrolidinone, in the presence of base, e.g. potassium carbonate, caesium carbonate, or sodium hydride. Other salts may aid the reaction, for example, sodium iodide or potassium iodide. Reaction conditions may vary from 40′-65° C. for periods ranging from 10 to 30 hours, typically overnight. This reaction is particularly useful when R¹ and R² are both C₁-C₃ alkyl or R¹ and R² together with the carbon atom to which they are attached form a 3- to 6-membered saturated carbocylic ring.

1.3 Reduction of an Alkyne

As shown in Scheme C, compounds of formula (I) wherein R¹ and R² are both C₁-C₃ alkyl and A-B is CH₂—CH₂ may be prepared from alkynes of formula (XIII) using standard reducing agents, such as hydrogen in the presence of a metal catalyst such as Wilkinson's catalyst, palladium on carbon or platinum oxide in a protic solvent, for example methanol, or those described in “Handbook of Reagents for Organic Synthesis—Oxidising and Reducing Agents” edited by S. D. Burke and R. L. Danheiser.

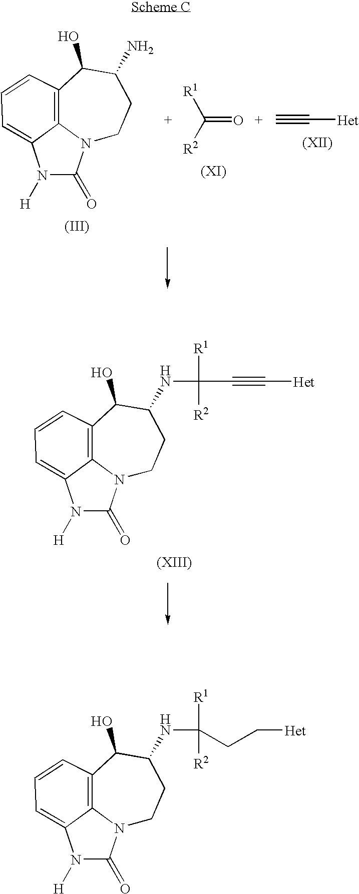

The alkynes of formula (XIII) may be prepared by reaction of the amino-alcohol of formula (III) with ketones of formula (XI) and the alkynes of formula (XII) in a suitable solvent such as methanol in the presence of a base, typically triethylamine, and cuprous bromide by heating in a sealed tube in a microwave oven at temperatures ranging from 100° C. to 125° C. for 0.5 to 3 hours, typically 45 minutes.

2. Preparation of Tricyclic Intermediates

2.1 Aminoalcohol (III)

The amino-alcohol of formula (III) may be prepared as shown in Scheme D. 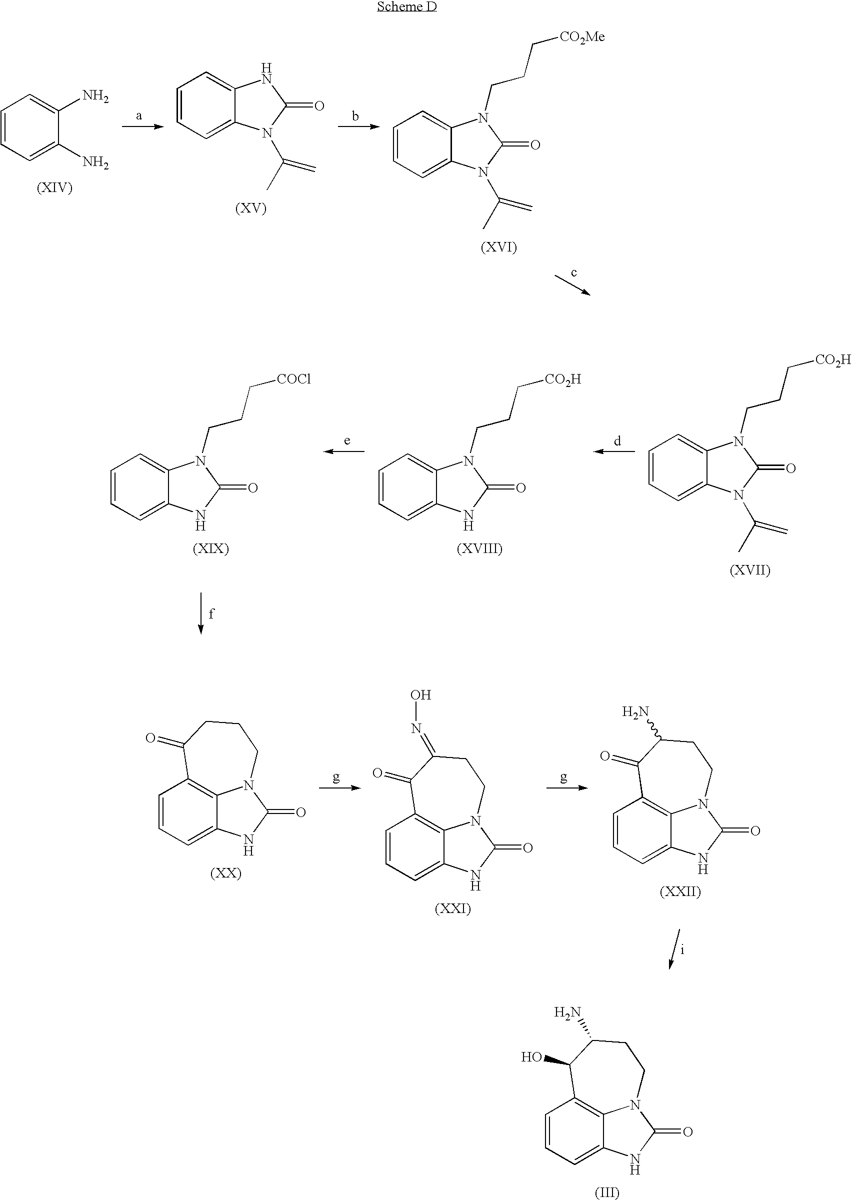
a) Ethyl acetoacetate, xylenes, 150° C.; b) 4-Bromobutyric acid methyl ester, K₂CO₃, acetone, reflux; c) 15% NaOH, THF, reflux; d) Conc. HCl, THF; e) SOCl₂, DCM; f) AlCl₃, DCM, reflux; g) t-BuONO, HCl, AcOH, 40° C.; h) Pd/C, H₂, MeOH, conc. HCl, 1.5 atm; i) NaBH₄, MeOH, 0° C.;

The preparation of the compounds of formula (XV), (XVI), (XVII), (XVIII), (XIX) and (XX) is disclosed in Tetrahedron Letters, 1995, 36, 9, 1387. The preparation of the compounds of formula (XXI) and (III) is disclosed in U.S. Pat. No. 4,585,770.

The enantiomers of the amino-alcohol (III) may be separated by chiral HPLC. N-protection facilitates the separation. Those skilled in the art will appreciate that a variety of N-protected compounds may be used, for example, the t-butyloxycarbamate prepared by reacting the amino-alcohol (III) with t-BOC-anhydride in a suitable solvent such as methanol, in the presence of a base such as triethylamine. Following chiral HPLC separation, the t-BOC protecting group may be removed by acid hydrolysis, for example, stirring in 4N HCl/dioxane at room temperature for several hours, typically 1 hour.

The desired enantiomer of the amino-alcohol (III) may also be prepared by the enantioselective reduction of the keto-oxime (XXI). Those skilled in the art will appreciate that the degree of enantioselectivity will depend on the catalyst, ligand, solvent and reaction temperature. Particularly useful conditions use hydrogen in the presence of a metal catalyst such as rhodium chloro(norbornadiene) dimer complexed with a ligand such as 1-[(S)-ferrocenyl-2-(R)-ethyl-1-dimethylamino)phenyl]-(S)-phosphino-1′-dicyclohexylphosphino-ferrocene (Solvias) in a protic solvent, typically aqueous methanol, at elevated temperatures, normally 80° C., for 10-40 hours, typically 16 hours.

3. Preparation of Ketones (II)

Many of the ketones of formula (II) and aldehydes of formula (VII) used in the reductive amination procedure are commercially available. Those skilled in the art will appreciate that others may be prepared by experimental procedures as described in the literature.

3.1 Compounds wherein A-B is CH═CH, C₁-C₃ alkyl=CH₃

Enones of formula (VI II) wherein C₁-C₃ alkyl ═CH₃, may be prepared according to the method illustrated in Scheme E from aldehydes of formula (XXIV), wherein Het is as defined for formula (I), by a base catalysed condensation with acetone, typically using sodium hydroxide, as base, at 0° C.

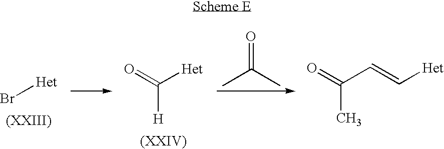

Substituted aldehydes of formula (XXIII), can be obtained by lithiation of the heteroaryl bromides (XXIII) using, for example, n-butyl lithium in tetrahydrofuran, followed by reaction of the aryl lithium reagent with N,N-dimethylformamide. The skilled person will recognise which heterocycles will be compatible with this reaction.

Alternatively, enones of formula (VIII) wherein C₁-C₃ alkyl ═CH₃, may be prepared from aldehydes of formula (XXIV) by reaction with 1-(triphenylphosphoranylidene)acetone in a suitable solvent, such as tetrahydrofuran, at elevated temperatures, normally reflux temperature, for 5-30 hours, typically overnight. Alternatively, enones of formula (VIII) wherein C₁-C₃ alkyl ═CH₃, may be prepared from aldehydes of formula (XXIV) by addition of sodium hydride (60% dispersion in oil) to diethyl (2-oxopropyl)phosphate in a suitable aprotic solvent, such as tetrahydrofuran; followed by dropwise of an aldehyde of formula (XXIV) at reduced temperature, typically 0° C. After reagent addition, the reaction may be stirred at room temperature for 5-30 hours, typically 18 hours.

Many heteroaryl aldehydes of formula (XXIV) are commercially available or may be prepared by procedures well known to those skilled in the art or described in the literature.

3.2 Compounds wherein A-B is CH═CH

Enones of formula (VIII) may be prepared according to the method illustrated in Scheme F by partial hydrogenation of the alkynes of formula (XXVII) using hydrogen in the presence of a Lindlar catalyst or other methods as described in “Handbook of Reagents for Organic Synthesis—Oxidising and Reducing Agents” edited by S. D. Burke and R. L. Danheiser. These alkynes may be prepared, for example, by the reaction of the organolithium reagents of formula (XXVI) with the N,N-dimethylamides of formula (XXV). 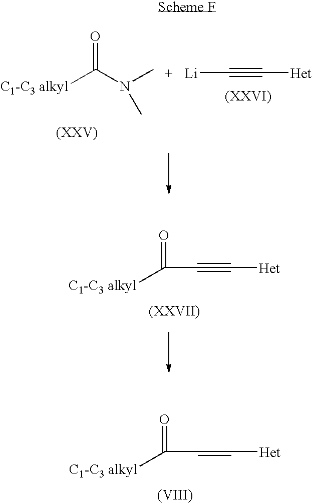
3.3 Compounds wherein A-B is CH₂—CH₂

Ketones of formula (II) wherein A-B is CH₂—CH₂ may be prepared from enones of formula (VIII) wherein Het is as defined for formula (I) using standard reducing agents, such as hydrogen in the presence of a metal catalyst such as Wilkinson's catalyst, palladium on alumina in a suitable solvent, for example ethyl acetate or methanol, or those described in “Handbook of Reagents for Organic Synthesis—Oxidising and Reducing Agents” edited by S. D. Burke and R. L. Danheiser, as illustrated in Scheme G.

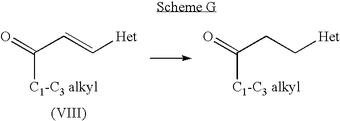

When Het is an optionally substituted fused pyrrole, then a particularly useful reaction is the nucleophilic addition to vinyl ketones as illustrated in Scheme H. Ketones of formula (XXX), wherein R³ and R⁴ are selected from the list of substituents as defined in formula (I) for the substitution of Het, may be prepared by the reaction of compounds of formula (XXVIII) with vinyl ketones of formula (XXIX) in a suitable solvent, such as dichloromethane using a Lewis acid catalyst, such as indium trichloride, at 0-20° C., typically room temperature.

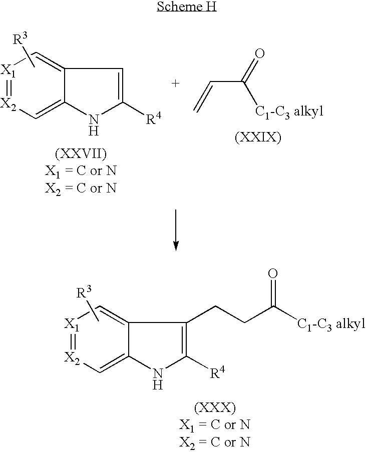

As shown in Scheme I, ketones of formula (XXXII) may be prepared by the reaction of the appropriate heterocycles of formula (XXXI) with 3-buten-2-one in a suitable solvent, such as dichloromethane, in the presence of a metal catalyst, such as zirconium (IV) chloride, at room temperature for 10-25 hours, typically 16 hours.

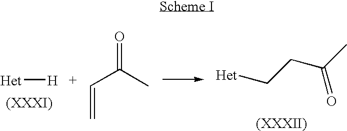

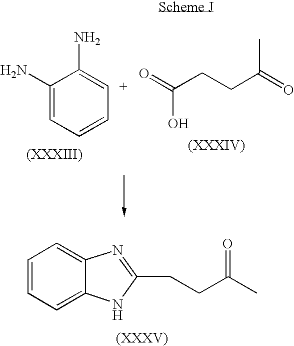

Specifically, as shown in Scheme J, the ketone of formula (XXXV) may be prepared by the reaction of benzene-1,2-diamine with 4-oxo-pentanoic acid by refluxing in 6N hydrochloric acid for 10-25 hours, typically 18 hours.

Ketones of formula (II) wherein A-B is CH₂—CH₂ may also be prepared by Heck coupling of the iodo compounds (XXXVI) with but-3-en-2-ol using Pd(OAc)₂ as catalyst in a suitable solvent, such as N,N-dimethylformamide, in the presence of a base, such as triethylamine, with optionally added inorganic salts, such as lithium chloride, as illustrated in Scheme K. 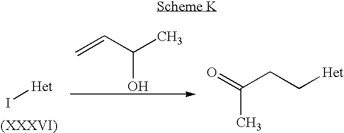
3.4 Compounds wherein B is —CR^{A}R^{B}—O—, —O—CR^{A}R^{B}—, —O—CR^{A}R^{B}—CR^{C}R^{D}—, —CR^{A}R^{B}—O—CR^{C}R^{D}—, or CR^{A}R^{B}—CR^{C}R^{D}—O—

Where chemically possible, the desired ketones or aldehydes may be prepared by reaction sequences illustrated in Scheme L.

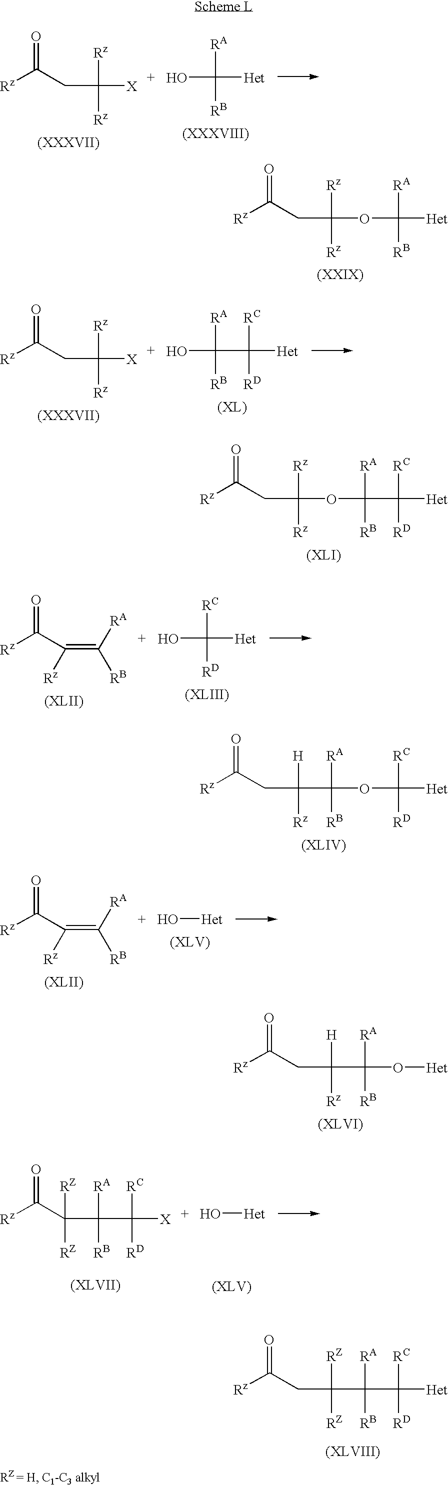

In the reaction sequences in Scheme L, X is a leaving group, typically 1, Br, Cl, OTs, OTf, O-mesylate, or O-trichloromethylsulphonate, preferably Br.

Ketones or aldehydes of formula (XXXIX) may be prepared by the reaction of the alcohols of formula (XXXVIII) (or the corresponding alcoholate anion) with the bromo-ketones or bromoaldehydes of formula (XXXVII) in a suitable solvent, e.g. dichloromethane, acetonitrile, dimethylformamide or N-methylpyrrolidinone, in the presence of base, e.g. triethylamine, potassium carbonate, caesium carbonate, or sodium hydride. Similarly compounds of formula (XLI) may be prepared from compounds of formula (XL).

Ketones or aldehydes of formula (XLIV) may be prepared by the nucleophilic addition of compounds of formula (XLIII) to compounds of formula (XLII). Similarly compounds of formula (XLVI) may be prepared by the nucleophilic addition of compounds of formula (XLV) to compounds of formula (XLII). The skilled person will recognise that a variety of standard literature experimental procedures may be used for these transformations. The skilled person will also recognise the limitations in the scope of these reactions.

4 Preparation of Reactants for Alkylation of Amino-Alcohol (III)

Compounds of formula (X) are required for the alkylation procedures. These can be prepared by the procedures illustrated in Scheme M.

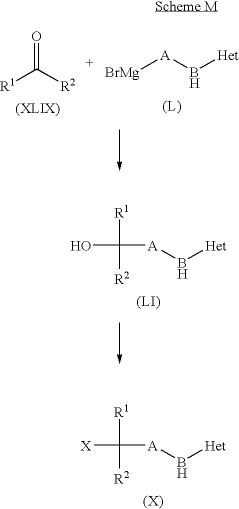

The alcohols of formula (LI) may be prepared by the addition of the Grignard reagents of formula (L) to the ketones/aldehydes of formula (XLIX) using standard literature Grignard reaction conditions. The required leaving group, X, may be prepared from the corresponding alcohol using standard functional group interconversion reactions known to those skilled in the art or as described in the literature. For example, X═Cl may be prepared by reaction with thionyl chloride and X═OMes may be prepared by reaction with mesyl chloride in a suitable solvent, such as dichloromethane, in the presence of a base.

5 Miscellaneous Transformations

Indole aldehydes of formula (LIV), wherein R³ is selected from the list of substituents as defined in formula (I) for the substitution of Het, may be prepared as shown in Scheme N.

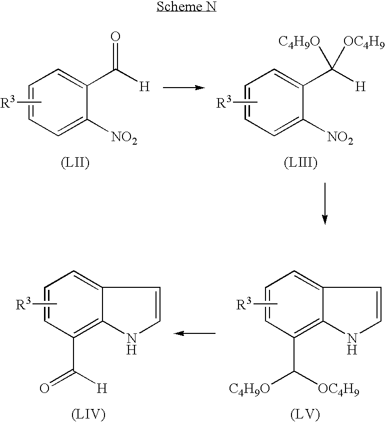

Ortho-nitrobenzaldehydes of formula (LII) can be protected as the acetals, (LIII), by reaction with n-butanol in refluxing toluene with an acid catalyst, such as para-toluenesulphonic acid, for 2-18 hours, typically 4 hours. The indoles of formula (LV) may be obtained by dropwise addition of a solution of vinylmagnesium bromide to the nitroacetals, (LIII), in a suitable solvent, such as tetrahydrofuran, at −70° C.

Deprotection of the acetals, (LV) to give the aldehydes, (LIV) may be achieved using standard conditions, for example, with a suitable acid such as hydrochloric acid in a solvent such as tetrahydrofuran.

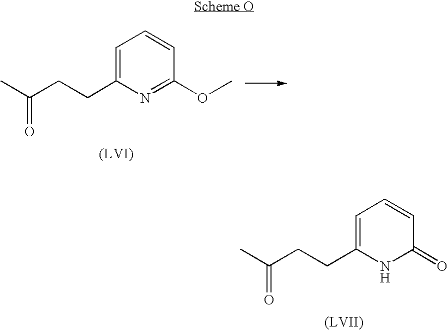

As shown in Scheme O, the ether of formula (LVI) may be demethylated by reaction with trimethylsilyl iodide by refluxing in a suitable solvent, such as trimethylsilyl iodide, for several hours, typically 2 hours.

In the synthesis of compounds of formula (I), wherein one or more of the substituents on the heterocyclic ring, where chemically feasible, is NH₂, the 2,5-dimethylpyrrole moiety is a useful protecting group for the amine during the transformations required in the synthesis of such compounds of formula (I) as depicted in Scheme P.

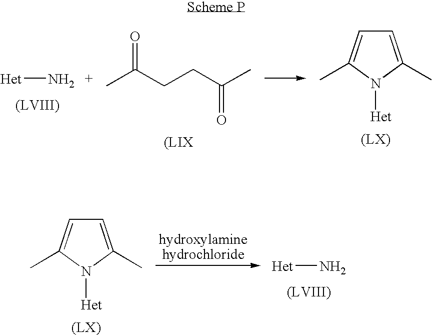

The pyrroles of formula (LX) may be prepared from the heterocyclic amines of formula (LVIII) by reaction with hexane-2,5-dione by heating at reflux using a Dean-Stark apparatus using a suitable solvent, such as toluene in the presence of an acid catalyst, such as p-toluenesulphonic acid, for 10-30 hours, typically 18 hours. The pyrroles of formula (LX) may be deprotected by reaction with hydroxylamine hydrochloride in a suitable solvent, such as ethanol, at elevated temperatures, typically 70° C. for several days, typically 7 days.

Aldehydes of formula (XXIV) may prepared from the acids of formula by the reduction/oxidation sequence shown in Scheme Q.

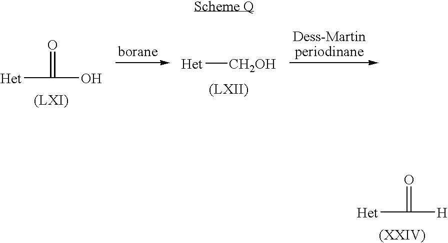

The alcohols of formula (LXII) may be prepared from the acids of formula (LXI) using standard reducing agents, such as borane in a suitable dipolar aprotic solvent, such as tetrahydrofuran, or those described in “Handbook of Reagents for Organic Synthesis—Oxidising and Reducing Agents” edited by S. D. Burke and R. L. Danheiser For reduction using borane, reagent addition is conducted in an inert atmosphere at reduced temperature, normally −5° C., followed by stirring the reaction mixture at room temperature for 10-25 hours, typically 18 hours. The aldehydes of formula (LXII) may be prepared from the alcohols of formula (LXI) using standard oxidising agents, such as Dess-Martin periodinane in a suitable solvent, such as dichloromethane, at room temperature under an inert atmosphere, or those described in “Handbook of Reagents for Organic Synthesis—Oxidising and Reducing Agents” edited by S. D. Burke and R. L. Danheiser.

It will also be appreciated by persons skilled in the art that, within certain of the processes described, the order of the synthetic steps employed may be varied and will depend inter alia on factors such as the nature of other functional groups present in a particular substrate, the availability of key intermediates, and the protecting group strategy (if any) to be adopted. Clearly, such factors will also influence the choice of reagent for use in the said synthetic steps.

The skilled person will appreciate that the compounds of the invention could be made by methods other than those herein described, by adaptation of the methods herein described and/or adaptation of methods known in the art, for example the art described herein, or using standard textbooks such as “Comprehensive Organic Transformations—A Guide to Functional Group Transformations”, R. C. Larock, Wiley-VCH (1999 or later editions).

It is to be understood that the synthetic transformation methods mentioned herein are exemplary only and they may be carried out in various different sequences in order that the desired compounds can be efficiently assembled. The skilled chemist will exercise his judgement and skill as to the most efficient sequence of reactions for synthesis of a given target compound.

In a preferred embodiment of the compounds of formula (I), R^{A}, R^{B}, R^{C}, R^{D}, R^{E} and R^{F} are each independently H or methyl. In another preferred embodiment of the compounds of formula (I), A is CH₂ and B is a covalent bond, CH₂ or C(CH₃)₂, or -A-B— is —CH═CH—. In another preferred embodiment, A is CH₂ and B is CH₂.

When -A-B— is —CH═CH— then the double bond preferably has the trans- (or E-) configuration.

In another preferred embodiment of the compounds of formula (I), R¹ and R² are each independently H or methyl. More preferably, one of R¹ and R² is H and the other is methyl. Yet more preferably, R¹ is H and R² is methyl such that the compound of formula (I) has the 1′R,6R,7R relative configuration. Most preferably the compound of formula (I) has the 1′R,6R,7R absolute configuration.

In another preferred embodiment of the compounds of formula (I), Het is selected from 5-membered monocyclic heteroaryl groups selected from furyl (including 2-furyl), pyrazolyl, imidazolyl (including 1-imidazolyl), oxazolyl, thiazolyl, isothiazolyl, triazolyl (including 1,2,4-triazolyl) and thiadiazolyl; 6-membered monocyclic heteroaryl groups selected from pyridyl (including 2-pyridyl, 3-pyridyl and 4-pyridyl) and pyridinonyl (including 2(1H)-pyridinonyl, such as 2(1H)-pyridinon-3-yl and 2(1H)-pyridinon-6-yl) and 9-membered fused bicyclic heteroaryl groups selected from indolyl (including 3-indolyl, 5-indolyl and 7-indolyl), benzofuryl, indazolyl, benzimidazolyl and pyrrolopyridinyl (including pyrrolo[3,2-b]pyridinyl and pyrrolo[2,3-c]pyridinyl).

In another preferred embodiment of the compounds of formula (I), when Het is substituted, the substituents may independently be selected from halo (including bromo, chloro and fluoro), —CN, (C₁-C₄)alkyl (including methyl), —OH, —O—(C₁-C₄ alkyl) (including O-methyl), —NH(C₁-C₄ alkyl) (including NH-methyl), —CO₂H, —CO₂(C₁-C₄ alkyl) (including CO₂Et), —CH₂Ph, —O—CH₂Ph and —NH₂.

In another preferred embodiment of the compounds of formula (I), Het is selected from furyl, pyrazolyl, imidazolyl, oxazolyl, thiazolyl, isothiazolyl, triazolyl, thiadiazolyl, pyridyl, pyridinonyl, indolyl, benzofuryl, indazolyl, benzimidazolyl and pyrrolopyridinyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

In another preferred embodiment of the compounds of formula (I), Het is selected from pyrazolyl, imidazolyl, thiazolyl, isothiazolyl, pyridyl, indolyl and pyrrolopyridinyl, and especially Het is selected from pyrazolyl, thiazolyl, isothiazolyl and pyridyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

When Het is pyrazolyl, it is preferably substituted with up to three (C₁-C₄)alkyl groups, for example three methyl groups. When Het is thiazolyl or isothiazolyl, it is preferably unsubstituted. When Het is pyridyl, it is preferably substituted with up to three —NH₂ groups, for example one NH₂ group.

In another preferred embodiment of the compounds of formula (I), Het is selected from imidazolyl, thiazolyl, indolyl, azaindolyl (also known as pyrrolopyridinyl) and benzimidazolyl, each of which may optionally be substituted with up to 3 groups independently selected from halo, —CN, (C₁-C₄)alkyl, —OH, —O—(C₁-C₄ alkyl), —O—CH₂—(C₃-C₅)cycloalkyl, —NH(C₁-C₄ alkyl), —CO₂H and —CO₂(C₁-C₄ alkyl).

Another preferred embodiment is a compound of formula (I^{A}) 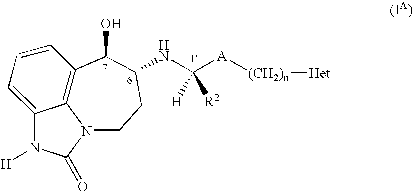
or a pharmaceutically acceptable salt thereof, wherein n is 0, 1 or 2, R² is H or methyl, and Het is selected from imidazolyl, thiazolyl, indolyl, azaindolyl and benzimidazolyl, each of which may optionally be substituted with up to 3 groups independently selected from halo, —CN, (C₁-C₄)alkyl, —OH, —O—(C₁-C₄ alkyl), —O—CH₂—(C₃-C₅)cycloalkyl, —NH(C₁-C₄ alkyl), —CO₂H and —CO₂(C₁-C₄ alkyl).

Another preferred embodiment is a compound of formula (I^{A}) or a pharmaceutically acceptable salt thereof, wherein n is 0, 1 or 2, R² is H or methyl, and Het is selected from furyl, pyrazolyl, imidazolyl, oxazolyl, thiazolyl, isothiazolyl, triazolyl, thiadiazolyl, pyridyl, pyridinonyl, indolyl, benzofuryl, indazolyl, benzimidazolyl and pyrrolopyridinyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

Another preferred embodiment is a compound of formula (I^{A}) or a pharmaceutically acceptable salt thereof, wherein n is 0, 1 or 2, R² is H or methyl, and Het is selected from pyrazolyl, imidazolyl, thiazolyl, isothiazolyl, pyridyl, indolyl and pyrrolopyridinyl, and especially Het is selected from pyrazolyl, thiazolyl, isothiazolyl and pyridyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

When Het is pyrazolyl, it is preferably substituted with up to three (C₁-C₄)alkyl groups, for example three methyl groups. When Het is thiazolyl or isothiazolyl, it is preferably unsubstituted. When Het is pyridyl, it is preferably substituted with up to three —NH₂ groups, for example one NH₂ group.

Another preferred embodiment is a compound of formula (I^{A}) or a pharmaceutically acceptable salt thereof that has the 6R,7R absolute configuration.

Another preferred embodiment is a compound of formula (I^{B}) 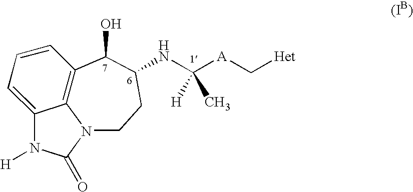
or a pharmaceutically acceptable salt thereof, wherein Het is indolyl optionally substituted by one or two groups selected from halo, —CN, (C₁-C₄)alkyl, —CH₂Ph, —OH, —O—(C₁-C₄ alkyl), —O—CH₂—(C₃-C₆)cycloalkyl, —O—CH₂Ph, —CO₂H and —CO₂(C₁-C₄ alkyl).

Another preferred embodiment is a compound of formula (I^{B}) or a pharmaceutically acceptable salt thereof, wherein Het is selected from furyl, pyrazolyl, imidazolyl, oxazolyl, thiazolyl, isothiazolyl, triazolyl, thiadiazolyl, pyridyl, pyridinonyl, indolyl, benzofuryl, indazolyl, benzimidazolyl and pyrrolopyridinyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

Another preferred embodiment is a compound of formula (I^{B}) or a pharmaceutically acceptable salt thereof, wherein Het is selected from pyrazolyl, imidazolyl, thiazolyl, isothiazolyl, pyridyl, indolyl and pyrrolopyridinyl, and especially Het is selected from pyrazolyl, thiazolyl, isothiazolyl and pyridyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂. When Het is pyrazolyl, it is preferably substituted with up to three (C₁-C₄)alkyl groups, for example three methyl groups. When Het is thiazolyl or isothiazolyl, it is preferably unsubstituted. When Het is pyridyl, it is preferably substituted with up to three —NH₂ groups, for example one NH₂ group. Another preferred embodiment is a compound of formula (I^{B}) or a pharmaceutically acceptable salt thereof that has the 1′R,6R,7R absolute configuration.

In embodiments of the compounds of formula (I), (I^{A}) and (I^{B}) wherein a substituent on Het is halo then preferably it is fluoro or chloro. In embodiments of the compounds of formula (I), (I^{A}) and (I^{B}) wherein a substituent on Het is (C₁-C₄)alkyl then preferably it is methyl, ethyl, propyl or isopropyl, and more preferably it is methyl. In embodiments of the compounds of formula (I), (I^{A}) and (I^{B}) wherein a substituent on Het is —O—(C₁-C₄)alkyl then preferably it is methoxy, ethoxy, propoxy or isopropoxy, and more preferably it is methoxy. In embodiments of the compounds of formula (I), (I^{A}) and (I^{B}) wherein a substituent on Het is —O—CH₂—(C₃-C₅)cycloalkyl then preferably it is cyclopropylmethoxy.

Preferred individual compounds of formula (I) are:
(6R*,7R*)-7-hydroxy-6-{[(1R*)-3-(1H-indol-3-yl)-1-methylpropyl]amino}-4,5,6,7-tetrahydroimidazo[4,5,1-jk][1]benzazepin-2(1H)-one;
(6R*,7R*)-7-hydroxy-6-{[(1S*)-3-(1H-indol-3-yl)-1-methylpropyl]amino}-4,5,6,7-tetrahydroimidazo[4,5,1-jk][1]benzazepin-2(1H)-one;
(6R,7R)-7-hydroxy-6-{[(1RS)-3-(1H-indol-3-yl)-1-methylpropyl]amino}-4,5,6,7-tetrahydroimidazo[4,5,1-jk][1]benzazepin-2(1H)-one;
(6R,7R)-7-hydroxy-6-{[(1R)-3-(1H-indol-3-yl)-1-methylpropyl]amino}-4,5,6,7-tetrahydroimidazo[4,5,1-jk][1]benzazepin-2(1H)-one;
(6R,7R)-7-hydroxy-6-{[(1S)-3-(1H-indol-3-yl)-1-methylpropyl]amino}-4,5,6,7-tetrahydroimidazo[4,5,1-jk][1]benzazepin-2(1H)-one;
(6R*,7R*)-6-{[(1R*)-3-(5-fluoro-1H-indol-3-yl)-1-methylpropyl]amino}-7-hydroxy-4,5,6,7-tetrahydroimidazo[4,5,1-jk][1]benzazepin-2(1H)-one;
(6R*,7R*)-6-{[(1S*)-3-(5-fluoro-1H-indol-3-yl)-1-methylpropyl]amino}-7-hydroxy-4,5,6,7-tetrahydroimidazo[4,5,1-jk][1]benzazepin-2(1H)-one;
(6R,7R)-6-{[(1RS)-3-(5-fluoro-1H-indol-3-yl)-1-methylpropyl]amino}-7-hydroxy-4,5,6,7-tetrahydroimidazo[4,5,1-jk][1]benzazepin-2(1H)-one;
(6R,7R)-6-{[(1R)-3-(5-fluoro-1H-indol-3-yl)-1-methylpropyl]amino}-7-hydroxy-4,5,6,7-tetrahydroimidazo[4,5,1-jk][1]benzazepin-2(1H)-one;
(6R,7R)-6-{[(1S)-3-(5-fluoro-1H-indol-3-yl)-1-methylpropyl]amino}-7-hydroxy-4,5,6,7-tetrahydroimidazo[4,5,1-jk][1]benzazepin-2(1H)-one;
(6R*,7R*)-6-{[(1R*)-3-(5-fluoro-1H-indol-7-yl)-1-methylpropyl]amino}-7-hydroxy-4,5,6,7-tetrahydroimidazo[4,5,1-jk][1]benzazepin-2(1H)-one;
(6R*,7R*)-6-{[(1S*)-3-(5-fluoro-1H-indol-7-yl)-1-methylpropyl]amino}-7-hydroxy-4,5,6,7-tetrahydroimidazo[4,5,1-jk][1]benzazepin-2(1H)-one;
(6R,7R)-6-{[(1RS)-3-(5-fluoro-1H-indol-7-yl)-1-methylpropyl]amino}-7-hydroxy-4,5,6,7-tetrahydroimidazo[4,5,1-jk][1]benzazepin-2(1H)-one;
(6R,7R)-6-{[(1R)-3-(5-fluoro-1H-indol-7-yl)-1-methylpropyl]amino}-7-hydroxy-4,5,6,7-tetrahydroimidazo[4,5,1-jk][1]benzazepin-2(1H)-one; and
(6R,7R)-6-{[(1S)-3-(5-fluoro-1H-indol-7-yl)-1-methylpropyl]amino}-7-hydroxy-4,5,6,7-tetrahydroimidazo[4,5,1-jk][1]benzazepin-2(1H)-one.

Especially preferred individual compounds of formula (I) are:
(6R*,7R*)-7-hydroxy-6-{[(1R*)-1-methyl-3-(1,3-thiazol-5-yl)propyl]amino}-4,5,6,7-tetrahydroimidazo[4,5,1-jk][1]benzazepin-2(1H)-one;
(6R*,7R*)-7-hydroxy-6-{[(1S*)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1RS)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1R)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1S)-1-methyl-3-(1,3-thiazol-5-yl)propyl]amino}-4,5,6,7-tetrahydroimidazo[4,5,1-jk][1]benzazepin-2(1H)-one;
(6R*,7R*)-7-hydroxy-6-{[(1R*)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R*,7R*)-7-hydroxy-6-{[(1S*)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1RS)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1R)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1S)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R*,7R*)-7-hydroxy-6-{[(1R*)-3-isothiazol-4-yl-1-methylpropyl]amino}-4,5,6,7-tetrahydroimidazo[4,5,1-jk][1]benzazepin-2(1H)-one;
(6R*,7R*)-7-hydroxy-6-{[(1S*)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1RS)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1R)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1S)-3-isothiazol-4-yl-1-methylpropyl]amino}-4,5,6,7-tetrahydroimidazo[4,5,1-jk][1]benzazepin-2(1H)-one;
(6R*,7R*)-6-{[(1R*)-3-(2-aminopyridin-3-yl)-1-methylpropyl]amino}-7-hydroxy-4,5,6,7-tetrahydroimidazo[4,5,1-jk][1]benzazepin-2(1H)-one;
(6R*,7R*)-6-{[(1S*)-3-(2-aminopyridin-3-yl)-1-methylpropyl]amino}-7-hydroxy-4,5,6,7-tetrahydroimidazo[4,5,1-jk][1]benzazepin-2(1H)-one;
(6R,7R)-6-{[(1RS)-3-(2-aminopyridin-3-yl)-1-methylpropyl]amino}-7-hydroxy-4,5,6,7-tetrahydroimidazo[4,5,1-jk][1]benzazepin-2(1H)-one;
(6R,7R)-6-{[(1R)-3-(2-aminopyridin-3-yl)-1-methylpropyl]amino}-7-hydroxy-4,5,6,7-tetrahydroimidazo[4,5,1-jk][1]benzazepin-2(1H)-one; and
(6R,7R)-6-{[(1S)-3-(2-aminopyridin-3-yl)-1-methylpropyl]amino}-7-hydroxy-4,5,6,7-tetrahydroimidazo[4,5,1-jk][1]benzazepin-2(1H)-one.

The compounds of formula (I) are agonists at the beta-2 adrenoceptor. In particular they have good efficacy at the bovine and/or porcine beta-2 adrenoceptor, as demonstrated in the assays set out below in the Examples.

The compounds of formula (I) may be used to improve meat production in livestock animals. Examples of livestock animals include ruminants such as cows, bulls, heifers, steers, goats, sheep and minor species such as buffalo, bison and antelopes. Other examples include pigs, boars, gilts, sows and avians such as chickens, ducks, geese and turkeys. A preferred use is in the improvement of meat production in cattle, swine and poultry.

Beta-2 agonists have also been reported to improve muscle production and feed efficiency in farmed fish. Accordingly, the compounds of formula (I) may find use in the production of fish such as, for example, tuna, salmon and trout.

The compounds of formula (I) may be administered to the animal by any suitable route. A preferred route of administration for improving meat production in livestock animals is the oral route. For such administration, the compounds of formula (I) may be added to the animals' food, drinking water, or any other material ingested by the animals, such as a salt lick.

The compounds of formula (I) may be added directly to the feed or drinking water, or may be presented as a concentrate for addition to the feed or drinking water.

The concentrate may be a solid or a liquid. Solid concentrates include simple mixtures of the compounds with a solid diluent such as corn starch, and compositions wherein the compounds are adsorbed onto the diluent. Examples of other diluents include alfalfa meal, rice hulls, corncob grits, bone meal, soybean meal, ground corn; inorganic diluents such as limestone, sodium chloride; vitamin and mineral mixes. Liquid concentrates include solutions and suspensions in water or another suitable vehicle, such as an oil, especially a vegetable oil.

A suitable concentrate for addition to feed comprises:

| | | | |
|---|---|---|---|
| Active agent | 0.1 to 2 | wt % | for example 0.5 wt % |
| Crushed limestone | 0.5 to 9 | wt % | for example 4.5 wt % |
| Rice hulls | 90 to 99 | wt % | for example 94.5 wt % |
| Mineral oil | 0.1 to 3 | wt % | for example 1 wt % |

The concentration of the compound of formula I in the feed or water should be adjusted such that each animal receives a maximally effective amount. For cattle, an intake of between 0.1 and 1000 mg/animal/day, particularly 0.1 to 100 mg/animal/day, may be suitable. Preferably the amount may be between 0.5 and 50 mg/animal/day, and more preferably between 1 and 25 mg/animal/day. For cattle consuming 10 kg of feed per day, this administration rate can be achieved by adding the compounds of formula I to the feed at an inclusion rate of 0.01 to 100 ppm, 0.01 to 10 ppm, 0.05 to 5 ppm, and 0.1 to 2.5 ppm.

Compounds of the present invention may be administered alone or in combination with one or more other compounds of the invention or in combination with one or more other drugs (or as any combination thereof).

For example, compounds of formula I may be used in combination with other feed additives used in livestock production; for example, polyether ionophores such as monensin, salinomycin, narasin, lasalocid and laidlomycin; antibiotics such as the tetracyclines, bacitracin, tylosin, tiamulin, lincomycin, virginiamycin, quinolone antibacterials and carbadox; melengesterol acetate; agents for the prevention or treatment of sub-acute rumen acidosis such as sodium bicarbonate, acarbose and other amylase or glucosidase inhibitors; carcass quality/anabolic agents such as ractopamine, salbutamol, almeterol and other beta adrenergic ligands; enzymes, minerals, vitamins and other supplements. The man skilled in the art will recognise that the agents listed above are examples of a wide range of feed additives which may be used in combination with compounds of formula I. Other examples are referred to in “2006 Feed Additive Companion” and “Handbook of Feed Additives 2006”.

Compounds of formula I may also be used in combination with anabolic agents such as zearanol, trenbolone acetate and oestradiol; and growth hormones such as bovine somatotropin and porcine somatotropin. Compounds of formula I may also be used in combination with agents used in animal welfare; for example endectocides such as ivermectin, doramectin, moxidectin, abamectin and other macrocyclic lactones; anthelmintics such as levamisole, albendazole and other benzimidazole carbamates, morantel, pyrantel; ectoparasiticides such as pyrethroids, arylpyrazoles, neonicotinoids.

Compounds of formula (I) may also be administered to livestock using other modes of oral administration, for example, as a bolus. Other agents, as listed above, may also be incorporated into the bolus. The bolus may be designed to reside in the rumen of a ruminant animal or in the stomach of a non-ruminant animal. The amount of active ingredient in such a bolus can be varied such that performance benefits may be observed over a part or the full lifetime of the animal and may also take into account any appropriate withholding periods.

Compounds of formula (I) may also be administered to livestock sub-cutaneously, for example, as an injectable implant. Such implants may also contain other agents such as an anabolic steroid together with suitable excipients. Preferably the site of injection will be in non-edible tissue, for example, in the ear in cattle.

The compounds of formula (I) may also be used in the treatment of diseases of animals in which beta-2 agonists have, or may have, a beneficial effect. In particular, the compounds of formula (I) may be used in the treatment of respiratory diseases of animals, including the treatment of heaves in horses.

The compounds of formula (I) also have agonist activity at the human beta-2 adrenoceptor and so are potentially useful in human medicine.

Beta-2 agonists are currently used to treat allergic and non-allergic airways diseases such as asthma and chronic obstructive airways disease (COPD). Treatment guidelines for these diseases include both short and long acting inhaled beta-2 agonists. Short acting, rapid onset beta-2 agonists are used for “rescue” bronchodilation, whereas, long-acting forms provide sustained relief and are used as maintenance therapy.

Bronchodilation is mediated via agonism of the beta-2 adrenoceptor expressed on airway smooth muscle cells, which results in relaxation and hence bronchodilation. Thus, as functional antagonists, beta-2 agonists can prevent and reverse the effects of all bronchoconstrictor substances, including leukotriene D4 (LTD4), acetylcholine, bradykinin, prostaglandins, histamine and endothelins. Because beta-2 receptors are so widely distributed in the airway, beta-2 agonists may also affect other types of cells that play a role in asthma. For example, it has been reported that beta-2 agonists may stabilize mast cells. The inhibition of the release of bronchoconstrictor substances may be how beta-2 agonists block the bronchoconstriction induced by allergens, exercise and cold air. Furthermore, beta-2 agonists inhibit cholinergic neurotransmission in the human airway, which can result in reduced cholinergic-reflex bronchoconstriction.

Therefore, a further aspect of the present invention relates to the compounds of formula (I), or pharmaceutically acceptable salts thereof, for use in the treatment of diseases, disorders, and conditions in which the beta-2 receptor is involved. More specifically, the present invention also concerns the compounds of formula (I), or pharmaceutically acceptable salts thereof, for use in the treatment of diseases, disorders, and conditions selected from the group consisting of:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension,
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
acute lung injury,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis.

In addition to the airways, it has also been established that beta-2 adrenoceptors are also expressed in other organs and tissues and thus the compounds of formula (I) may have application in the treatment of other diseases such as, but not limited to those of the nervous system, premature labor, congestive heart failure, depression, inflammatory and allergic skin diseases, psoriasis, proliferative skin diseases, glaucoma and in conditions where there is an advantage in lowering gastric acidity, particularly in gastric and peptic ulceration.

When used in human therapy, the compounds of formula (I) and their pharmaceutically acceptable salts will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLApoly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of formula (I). The overall daily dose will typically be in the range 0.001 mg to 40 mg which may be administered in a single dose or, more usually, as divided doses throughout the day.

The compounds of formula (I) are particularly suitable for an administration by inhalation.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 0.001 mg to 5000 mg depending, of course, on the mode of administration. For example, an intravenous daily dose may only require from 0.001 mg to 40 mg. The total daily dose may be administered in single or divided doses and may, at the physician's discretion, fall outside of the typical range given herein.

These dosages are based on an average human subject having a weight of about 65 kg to 70 kg. The physician will readily be able to determine doses for subjects whose weight falls outside this range, such as infants and the elderly.

When used for the treatment of human airway disease, the compounds of formula (I) and their pharmaceutically acceptable salts may advantageously be used in combination with a second pharmacologically active agent. Examples of such agents include: H3 antagonists, muscarinic M3 receptor antagonists, PDE4 inhibitors, glucocorticosteroids, adenosine A2a receptor agonists, modulators of cytokine signalling pathways such as p38 MAP kinase or syk kinase, and leukotriene antagonists (LTRAs) including antagonists of LTB₄, LTC₄, LTD₄, and LTE₄.

Particularly preferred agents for such combination therapy are:
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate, and
muscarinic M3 receptor antagonists or anticholinergic agents including in particular ipratropium salts, namely bromide, tiotropium salts, namely bromide, oxitropium salts, namely bromide, perenzepine, and telenzepine.

### EXAMPLES

The following non-limiting Examples illustrate the preparation of compounds of the formula (I).

In the following experimental details, nuclear magnetic resonance spectral data were obtained using Varian Inova 300, Varian Inova 400, Varian Mercury 400, Varian Unityplus 400, Bruker AC 300 MHz, Bruker AM 250 MHz or Varian T60 MHz spectrometers, the observed chemical shifts being consistent with the proposed structures. Key n.m.r. chemical shifts are quoted in p.p.m. downfield from tetramethylsilane. In the following Examples, where an Example is indicated as being a mixture of diastereoisomers, then the n.m.r. integrals shown refer to the relative ratio of integrals for the quoted chemical shift. Mass spectral data were obtained on a Finnigan Masslab Navigator, a Fisons Instrument Trio 1000, or a Hewlett Packard GCMS System Model 5971 spectrometer. The calculated and observed ions quoted refer to the isotopic composition of lowest mass. HPLC means high performance liquid chromatography. Where indicated, the following analytical HPLC methods have been used:

HPLC Method A:

Gilson system, 150×4.6 mm Gemini C18 5 μm column. HPLC linear gradient:

| | Pump A | Pump B | |
|---|---|---|---|
| | Acetonitrile/ | Acetonitrile/ | |
| Time | water (containing | water (containing | |
| minutes | 0.1% ammonia) (5:95) | 0.1% ammonia) (95:5) | Flow rate |
| 0 | 100% | 0% | 1 ml/min |
| 3 | 100% | 0% | 1 ml/min |
| 20 | 0% | 100% | 1 ml/min |
| 30 | 0% | 100% | 1 ml/min |

HPLC Method B:

Gilson system, 150×4.6 mm LUNA C18(2) 5 μm column. HPLC linear gradient:

| | Pump A | Pump B | |
|---|---|---|---|
| | Acetonitrile/ | Acetonitrile/ | |
| Time | ammonium formate | ammonium formate | |
| minutes | 20 mM (5:95) | 20 mM (98:2) | Flow rate |
| 0 | 100% | 0% | 1 ml/min |
| 1 | 100% | 0% | 1 ml/min |
| 10 | 0% | 100% | 1 ml/min |
| 30 | 0% | 100% | 1 ml/min |

HPLC Method C:
Gilson system, 250×4.6 mm Chiralcel OD-H 5 μm column;
Ethanol:hexane [20:80], 1 ml/min.
   HPLC Method D:
Gilson system, 250×4.6 mm ID Chiralpak AD-H 5 μm column;
Methanol:ethanol:hexane [5:15:80] with 0.1% v/v triethylamine, 1 ml/min.
   HPLC Method E:
Gilson system, 250×4.6 mm ID Chiralpak OD-H 5 μm column;
Ethanol: hexane [20:80] with 0.1% v/v triethylamine, 1 ml/min
   HPLC Method F:
Gilson system, 250×4.6 mm ID Chiralpak OD-H 5 μm column;
Ethanol:hexane [20:80], 1 ml/min.
   Biological Test

Compounds of the present invention have been found to display activity in cAMP assays selective for the bovine and porcine beta-2 adrenoceptors.

CHO cells transfected with the bovine or porcine beta-2 adrenceptors were maintained in culture in DMEM/HAMS F12+10% FBS+2 mM glutamine+500 μg/ml geneticin (for the porcine receptor the medium was supplement with 1.5 mM HEPES) at 37° C. with a 5% CO₂ atmosphere.

Cells were plated into 96 well viewplates in medium and incubated overnight at 37° C. with a 5% CO₂ atmosphere. The cells were pre-incubated with 0.5 mM IBMX in PBS for 30 minutes prior to incubation with increasing concentrations of experimental compound (5×10⁻¹² to 10⁻⁵M) for 30 minutes at 37° C. with a 5% CO₂ atmosphere. At the end of the incubation time the compound was removed and the cells assayed for cAMP using the DiscoveRx Hit Hunter cAMP II™ assay kit.

Duplicate samples were run for each experimental compound and the data generated was analysed using EC₅₀ analysis software in Graphpad Prism.

Room temperature means 20 to 25° C. N/A indicates no data available.

In the following Examples, structures are depicted as follows:

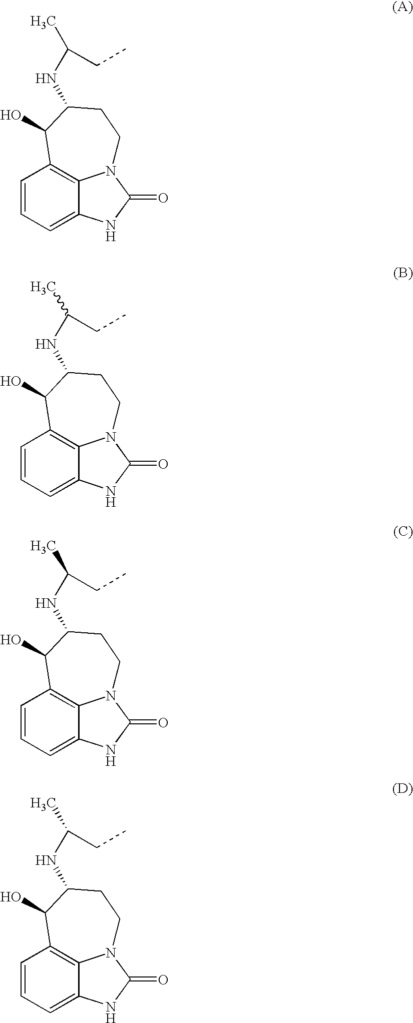

Unless specified otherwise, the wedge and dashed bonds indicate relative stereochemistry only. In particular, the 7-hydroxyl and the 6-N-substituent are oriented in a trans configuration, but the structures encompass both the 6R,7R and 6S,7S stereoisomers. Formula (A) represents a compound which is a mixture of epimers at the carbon atom bearing the methyl substituent. Formula (B) represents a compound which is a single, unidentified epimer at the carbon atom bearing the methyl substituent. Formulae (C) and (D) represent single epimers of known relative configuration. Thus, formula (A) represents a compound that is a mixture of (C) and (D), while (B) represents a compound that is either (C) or (D).

### Example 1

### 6-{[3-(5-Fluoro-1H-indol-7-yl)-1-methylpropyl]amino}-7-hydroxy-4,5,6,7-tetrahydroimidazo[4,5,1-jk][1]benzazepin-2(1H)-one

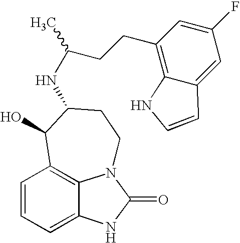

To a mixture of the compound of Preparation 1 (973 mg, 3.8 mmol) and the compound of Preparation 13 (820 mg, 4.0 mmol) in methanol (10 ml) was added triethylamine (0.2 ml, 1.1 mmol). After stirring for 30 min, sodium cyanoborohydride (359 mg, 5.7 mmol) was added and the reaction mixture was stirred at 50° C. for 5 h. The mixture was concentrated in vacuo and to the residue was added dichloromethane:methanol (9:1, 1 ml). This solution was purified by automated flash chromatography (Biotage™ 40M cartridge) with gradient elution, dichloromethane:2% methanolic ammonia [98:2 to 90:10]. The appropriate fractions were combined and concentrated to give the compound of Example 1a (290 mg) as a pair of enantiomers. HPLC Method A—retention time 15.13 min.

To a solution of the compound of Example 1a (468 mg, 1.2 mmol) in methanol (6 ml) was added dropwise hydrogen chloride in diethyl ether (1M, 3.5 ml). After stirring for 2 h, diethyl ether (20 ml) was added dropwise and the precipitate was collected by filtration. The resulting solid was washed with diethyl ether (2×20 ml) and dried in a vacuum oven to give the hydrochloride salt, the compound of Example 1b (436 mg), as a pair of enantiomers. HPLC Method A—retention time 15.05 min.

| Exam- | Structure | MH⁺ | MH⁺ | Bovine | Porcine |
|---|---|---|---|---|---|
| ple | Comment | found | expected | EC₅₀ nM | EC₅₀ nM |
| 1a | Second eluting pair | N/A | | 0.5 | 1.4 |
| | of enantiomers | | | | |
| | HPLC Method A | | | | |
| 1b | Second eluting pair | 409.3 | 409.2 | 0.7 | 1.0 |
| | of enantiomers - | | | | |
| | HPLC Method A - | | | | |
| | hydrochloride salt | | | | |

### Example 1a

¹H-NMR (CD₃OD): 1.15-1.20 (3H), 4.62-4.66 (1H), 6.35-6.39 (1H), 6.67-6.72 (1H), 6.97-7.03 (2H), 7.03-7.09 (1H), 7.16-7.24 (2H)

### Example 1b

¹H-NMR (CD₃OD): 1.47-1.51 (3H), 2.03-2.14 (2H), 4.92-4.95 (1H), 6.42-6.45 (1H), 6.77-6.83 (1H), 7.03-7.14 (3H), 7.28-7.32 (2H)

### Example 2

### 5-(3-{[7-Hydroxy-2-oxo-1,2,4,5,6,7-hexahydroimidazo[4,5,1-jk][1]benzazepin-6-yl]amino}butyl)-2-furoic acid

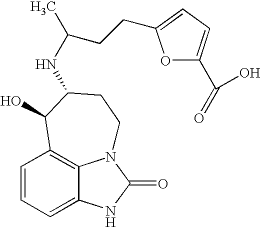

A mixture of the compound of Preparation 1 (117 mg, 0.5 mmol), triethylamine (100 μl, 0.7 mmol) and the compound of Preparation 156 (199 mg, 1.0 mmol) in methanol (2 ml) was heated at 80° C. in a microwave oven (300 W) for 40 min. The reaction mixture was stirred overnight at room temperature, before addition of sodium borohydride (120 mg, 3.2 mmol). After stirring at room temperature for 18 h, the mixture was diluted with methanol (8 ml) and Amberlyst® 15 ion-exchange resin (4 g, prepared according to J. Org. Chem. 1998, 63, 3471-3473) was added. The mixture was shaken overnight and the solution was filtered off. The resin was washed with methanol (3×20 ml) and treated with ammonia in methanol (2N, 15 ml). After shaking for 2 h, the solution was filtered off and the resin was washed with ammonia in methanol (2N, 2×15 ml). The combined methanol/ammonia washings were concentrated in vacuo and the residue was re-dissolved in methanol (5 ml). This solution was filtered and concentrated in vacuo. The residue was dissolved in acetonitrile:water (1:1, 1.4 ml) and purified by automated preparative liquid chromatography (Gilson system, 150 mm×19 mm XTERRA C18 5 μm column, 20 ml/min) using an acetonitrile: 0.1% aqueous ammonia (1:9): acetonitrile:0.1% aqueous ammonia (9:1) gradient [1:0 to 0:1 (over 20 min) then at 0:1 (for 5 min)]. The appropriate fractions were concentrated in vacuo to give the compound of Example 2 (26 mg) as a mixture of 4 diastereoisomers.

Experimental MH⁺ 385.5; expected 386.2

¹H-NMR (CD₃OD): 1.10-1.18 (3H), 4.62-4.66 (1H), 6.13-6.20 (1H), 6.94-7.00 (2H), 7.05-7.10 (1H), 7.15-7.20 (1H)

Bovine EC₅₀—171 nM; Porcine EC₅₀—31 nM

### Example 3

### 7-Hydroxy-6-{[1-methyl-3-(2-methyl-1H-indol-3-yl)propyl]amino}-4,5,6,7-tetrahydroimidazo[4,5,1-jk][1]benzazepin-2(1H)-one

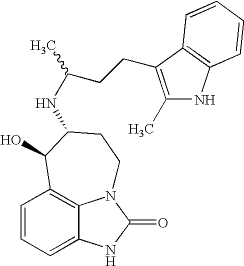

To a mixture of the compound of Preparation 1 (838 mg, 3.3 mmol) and the compound of Preparation 49 (770 mg, 3.8 mmol) in methanol (40 ml) was added triethylamine (0.2 ml, 1.1 mmol). After stirring for 1 h, sodium cyanoborohydride (361 mg, 5.7 mmol) was added and the reaction mixture was stirred at 50° C. for 60 h. The mixture was concentrated in vacuo and to the residue was added dichloromethane (20 ml) and methanol (0.5 ml). This solution was purified by automated flash chromatography (Biotage™ 65i cartridge conditioned with dichloromethane:2% methanolic ammonia [98:2]) with gradient elution, dichloromethane:2% methanolic ammonia [98:2 to 90:10]. The appropriate fractions were combined and concentrated to give the compound of Example 3a (533 mg) as a pair of enantiomers. HPLC Method A—retention time 14.74 min.

To a solution of the compound of Example 3a (530 mg, 1.3 mmol) in methanol (7.5 ml), at 0° C., was added dropwise hydrogen chloride in diethyl ether (1M, 1.3 ml). After stirring at 0° C. for 1 h, diethyl ether (42 ml) was added dropwise and the precipitate was collected by filtration. The resulting solid was washed with 15% methanol/diethyl ether (15 ml), followed by diethyl ether (2×15 ml), and dried in a vacuum oven at 50° C. to give the hydrochloride salt, the compound of Example 3b (499 mg), as a mixture of enantiomers. HPLC Method A—retention time 14.75 min.

Experimental MH⁺ 405.3; expected 405.2

¹H-NMR (CD₃OD): 1.42-1.46 (3H), 1.86-2.00 (2H), 2.35-2.37 (3H), 4.77-4.81 (1H), 6.90-7.02 (3H), 7.04-7.09 (1H), 7.16-7.20 (2H), 7.41-7.45 (1H)

Bovine EC₅₀—5.5 nM; Porcine EC₅₀—3.0 nM

### Example 4

### 7-Hydroxy-6-{[3-(1H-indol-3-yl)-1-methylpropyl]amino}-4,5,6,7-tetrahydroimidazo[4,5,1-jk][1]benzazepin-2(1H)-one

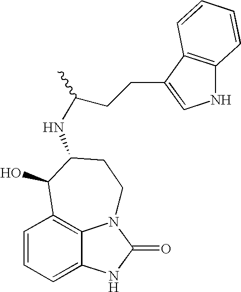

To a mixture of the compound of Preparation 1 (1.4 g, 5.3 mmol) and the compound of Preparation 43 (1.0 g, 5.3 mmol) in methanol (30 ml) was added triethylamine (1.9 ml, 13.4 mmol). After stirring for 60 h, the mixture was cooled to 0° C. and sodium borohydride (808 mg, 21.4 mmol) was added. After stirring for 15 min, the mixture was quenched with water (2 ml) and concentrated in vacuo. The residue was pre-absorbed on to silica (6 g) and purified by column chromatography (Isolute™ cartridge, 50 g) with gradient elution, dichloromethane:2% methanolic ammonia [100:0 to 90:10]. The appropriate fractions were combined and concentrated to give the compound of Example 4a (88 mg) as a mixture of 4 diastereoisomers.

To a solution of the compound of Example 4a (880 mg, 2.3 mmol) in methanol (10 ml) was added dropwise hydrogen chloride in diethyl ether (1M, 4.5 ml). After stirring for 18 h, diethyl ether (50 ml) was added dropwise and the precipitate was collected by filtration. The resulting solid was washed with 20% methanol/diethyl ether (2×30 ml), followed by diethyl ether (2×30 ml), and dried in a vacuum oven at 50° C. to give the hydrochloride salt, the compound of Example 4b (997 mg), as a mixture of 4 diastereoisomers.

A solution of the compound of Example 4a (3.2 g, 7.5 mmol) in 20% methanol dichloromethane (24 ml) was purified by automated flash chromatography (Biotage™ 65i cartridge conditioned with dichloromethane:2% methanolic ammonia [98:2]) with gradient elution, dichloromethane:2% methanolic ammonia) [98:2 to 80:20]. The appropriate fractions were combined and concentrated to give the compound of Example 4c (860 mg) as a pair of enantiomers. HPLC Method A—retention time 13.73 min. Other appropriate fractions were combined and concentrated to give the compound of Example 4d (746 mg) as a pair of enantiomers. HPLC Method A—retention time 14.45 min.

The compound of Example 4d (approximately 1.2 g, 3.1 mmol) was dissolved in ethanol (15 ml) and the enantiomers were separated by automated preparative liquid chromatography (Gilson system, 500×50 mm Chiralcel OD, 20 μm column, 50 ml/min) using methanol:ethanol:hexane [5:15:80] as the mobile phase. The appropriate fractions were combined and concentrated to give the compound of Example 4e (542 mg) as a single enantiomer. HPLC Method C—retention time 34.44 min.

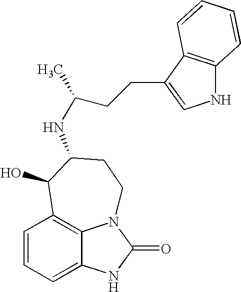

The compound of Example 4e—absolute stereochemistry

To a solution of the compound of Example 4e (524 mg, 1.3 mmol) in methanol (8 ml), at 0° C., was added dropwise hydrogen chloride in diethyl ether (1M, 1.5 ml). After stirring for 2 h, diethyl ether (40 ml) was added dropwise and the precipitate was collected by filtration. The resulting solid was washed with diethyl ether (40 ml), and dried in a vacuum oven at 50° C. to give the hydrochloride salt, the compound of Example 4f (480 mg). HPLC Method C—retention time 36.2 min.

| Exam- | Structure | MH⁺ | MH⁺ | Bovine | Porcine |
|---|---|---|---|---|---|
| ple | Comment | found | expected | EC₅₀ nM | EC₅₀ nM |
| 4a | Mixture of 4 | 391.1 | 391.2 | 3.2 | 6.9 |
| | diastereoisomers | | | | |
| 4b | Mixture of 4 | 391.1 | 391.2 | 3.8 | 6.9 |
| | diastereoisomers - | | | | |
| | hydrochloride salt | | | | |
| 4c | First eluting pair | 391.2 | 391.2 | 103 | 143 |
| | of enantiomers - | | | | |
| | HPLC Method A | | | | |
| 4d | Second eluting pair | 391.1 | 391.2 | 2.1 | 3.0 |
| | of enantiomers - | | | | |
| | HPLC Method A | | | | |
| 4e | Single enantiomer | 391.1 | 391.2 | 1.1 | 3.7 |
| 4f | Single enantiomer - | 391.1 | 391.2 | 1.7 | 2.5 |
| | hydrochloride salt | | | | |

### Example 4a

¹H-NMR (CD₃OD): 1.11-1.19 (3H), 1.58-1.81 (2H), 4.54-4.61 (1H), 6.85-7.05 (5H), 7.06-7.16 (1H), 7.23-7.29 (1H), 7.42-7.49 (1H)

### Example 4b

¹H-NMR (CD₃OD): 1.41-1.48 (3H), 1.90-2.10 (2H), 4.67-4.72 (1H), 6.93-7.10 (5H), 7.20-7.32 (2H), 7.44-7.56 (1H)

### Example 4c

¹H-NMR (CD₃OD): 1.23-1.28 (3H), 2.00-2.23 (2H), 4.60-4.64 (1H), 6.87-6.91 (1H), 6.97-7.06 (4H), 7.12-7.16 (1H), 7.29-7.33 (1H), 7.47-7.50 (1H)

### Example 4d

¹H-NMR (CD₃OD): 1.12-1.16 (3H), 1.64-1.81 (2H), 4.58-4.61 (1H), 6.90-6.98 (3H), 6.99-7.05 (2H), 7.12-715(1H), 7.24-7.28 (1H), 7.45-7.49 (1H)

### Example 4e

¹H-NMR (d₆-DMSO): 1.00-1.07 (3H), 4.51-4.56 (1H), 6.82-6.88 (1H), 6.89-6.95 (2H), 7.00-7.10 (3H), 7.25-7.30 (1H), 7.43-7.49 (1H)

### Example 4f

¹H-NMR (CD₃OD): 1.20-1.22 (3H), 1.90-2.05 (2H), 4.82-4.84 (1H), 6.92-7.01 (2H), 7.02-7.08 (3H), 7.20-7.23 (1H), 7.26-7.29 (1H), 7.55-7.57 (1H)

### Example 5

### Hydrochloride salt of 6-{[3-(5-Fluoro-1H-indol-3-yl)-1-methylpropyl]amino}-7-hydroxy-4,5,6,7-tetrahydroimidazo[4,51-jk][1]benzazepin-2(1H)-one

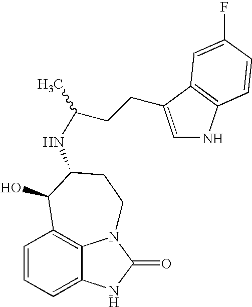

To a solution of the compound of Preparation 1 (1.0 g, 3.9 mmol) in methanol (20 ml) was added the compound of Preparation 50 (802 mg, 3.9 mmol), followed by triethylamine (0.2 ml, 1.2 mmol). After stirring for 30 min, sodium cyanoborohydride (614 mg, 9.8 mmol) was added and the reaction mixture was heated at 50° C. for 18 h. The mixture was concentrated in vacuo and the residue was dissolved in 10% methanol:dichloromethane (20 ml) and purified by automated flash chromatography (Biotage™ 65i cartridge, conditioned with dichloromethane:2.5% methanolic ammonia [97:3]) with gradient elution, dichloromethane:2.5% methanolic ammonia) [97:3 to 85:15]. The appropriate fractions were combined and concentrated to give the compound of Example 5a (725 mg) as a pair of enantiomers. HPLC Method A—retention time 14.48 min.

To a solution of the compound of Example 5a (718 mg, 1.8 mmol) in methanol (11 ml), at 0° C., was added dropwise hydrogen chloride in diethyl ether (1M, 2.0 ml). After stirring for 30 min, diethyl ether (65 ml) was added and the solution was allowed to stand for 30 min. The precipitate was collected by filtration and the resulting solid was washed with diethyl ether (4×25 ml) and dried in a vacuum oven at 50° C. to give the hydrochloride salt, the compound of Example 5b (671 mg), as a pair of enantiomers. HPLC Method A—retention time 14.47 min.

¹H-NMR (CD₃OD): 1.41-1.45 (3H), 1.96-2.08 (2H), 4.85-4.89 (1H), 6.80-6.86 (1H), 7.00-7.11 (2H), 7.14-7.16 (1H), 7.20-7.28 (3H)

Bovine EC₅₀—1.1 nM; Porcine EC₅₀—2.5 nM

### Example 6

### 7-Hydroxy-6-{[1-methyl-3-(1,3-thiazol-5-yl)propyl]amino}-4,5,6,7-tetrahydroimidazo[4,5,1-jk][1]benzazepin-2(1H)-one

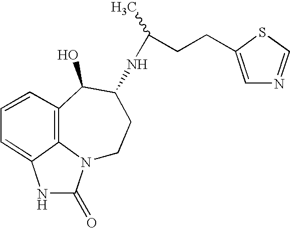

To a solution of the compound of Preparation 1 (300 mg, 1.2 mmol) in methanol (20 ml) was added the compound of Preparation 14 (219 mg, 1.4 mmol), followed by triethylamine (49 μl, 0.4 mmol). After stirring for 1 h, sodium cyanoborohydride (111 mg, 1.8 mmol) was added and the reaction mixture was heated at 60° C. for 18 h. The mixture was concentrated in vacuo and the residue was dissolved in acetonitile:water (9:1, 5 ml) and purified by automated preparative liquid chromatography (Gilson system, 150×21.4 mm Gemini C18(2) 5 μm column, 20 ml/min) using an acetonitrile:0.1% aqueous ammonia (5:95): acetonitrile:0.1% aqueous ammonia (95:5) gradient [90:10 to 70:30 (from 3 to 15 min) to 50:50 (from 20 to 25 min) to 5:95 (from 25 to 26 min)]. The appropriate fractions were combined and concentrated to give the compound of Example 6a (35 mg) as a pair of enantiomers. HPLC Method A—retention time 10.75 min. Other appropriate fractions were combined and concentrated to give the compound of Example 6b (83 mg) as a pair of enantiomers. HPLC Method A—retention time 11.06 min.

| Exam- | Structure | MH⁺ | MH⁺ | Bovine | Porcine |
|---|---|---|---|---|---|
| ple | Comment | found | expected | EC₅₀ nM | EC₅₀ nM |
| 6a | First eluting pair of | 359.4 | 359.2 | 22.9 | >10000 |
| | enantiomers - | | | | |
| | HPLC Method A | | | | |
| 6b | Second eluting pair | 359.4 | 359.2 | 3.6 | >10000 |
| | of enantiomers - | | | | |
| | HPLC Method A | | | | |

### Example 6a

¹H-NMR (CD₃OD): 1.15-1.19 (3H), 1.79-1.86 (2H), 4.64-4.66 (1H), 6.99-7.07 (2H), 7.17-7.19 (1H), 7.58-7.59 (1H), 8.80-8.81 (1H)

### Example 6b

¹H-NMR (CD₃OD): 1.14-1.18 (3H), 1.79-1.86 (2H), 4.63-4.66 (1H), 6.99-7.06 (2H), 7.20-7.23 (1H), 7.61-7.62 (1H), 8.80-8.81 (1H)

### Example 7

### 7-Hydroxy-6-{[1-methyl-3-(6-oxo-1,6-dihydropyridin-2-yl)propyl]amino}-4,5,6,7-tetrahydroimidazo[4,5,1-jk][1]benzazepin-2(1H)-one

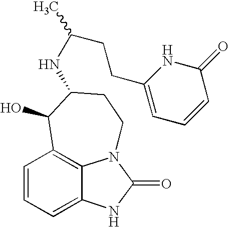

A mixture of the compound of Preparation 1 (117 mg, 0.5 mmol), triethylamine (100 μl, 0.7 mmol) and the compound of Preparation 67 (100 mg, 1.0 mmol) in methanol (2 ml) was heated at 80° C. in a microwave oven (300 W) for 40 min. The reaction mixture was stirred overnight at room temperature, before addition of sodium borohydride (120 mg, 3.2 mmol). After stirring at room temperature for 18 h, the mixture was diluted with methanol (8 ml) and Amberlyst® 15 ion-exchange resin (4 g, prepared according to J. Org. Chem. 1998, 63, 3471-3473) was added. The mixture was shaken overnight and the solution was filtered off. The resin was washed with methanol (3×20 ml) and treated with ammonia in methanol (2N, 15 ml). After shaking for 2 h, the solution was filtered off and the resin was washed with ammonia in methanol (2N, 2×15 ml). The combined methanol/ammonia washings were concentrated in vacuo and the residue was re-dissolved in methanol (5 ml). This solution was filtered and concentrated in vacuo. The residue was dissolved in acetonitrile:water (1:1, 1.2 ml) and purified by automated preparative liquid chromatography (Gilson system, 150 mm×21.2 mm Gemini 5 μm column, 20 ml/min) using an acetonitrile: 0.1% aqueous ammonia (1:9):acetonitrile:aqueous ammonia (9:1) gradient [100:0 to 20:80 (from 2 to 20 min) to 0:100 (from 20 to 25 min)]. The appropriate fractions were combined and concentrated to give the compound of Example 7a (35 mg) as a mixture of 4 diastereoisomers.

Alternative Synthesis

To a solution of the compound of Preparation 1 (212 mg, 0.8 mmol) in methanol (5 ml) was added the compound of Preparation 67 (192 mg, 1.2 mmol), followed by potassium hydroxide (56 mg, 1.0 mmol). The reaction mixture was stirred at room temperature for 10 min, before addition of sodium cyanoborohydride (78 mg, 1.2 mmol). After stirring for 60 h, the reaction mixture was cooled to 0° C. and sodium borohydride (47 mg, 1.2 mmol) was added. The mixture was stirred for a further 1 h and silica (1.5 g) was added, before the mixture was concentrated in vacuo. The silica/product mix was purified by automated flash chromatography (Biotage™ 40M cartridge conditioned with dichloromethane) with gradient elution, dichloromethane 2% methanolic ammonia [100:0 to 90:10]. The appropriate fractions were combined and concentrated to give the compound of Example 7b (98 mg) as a pair of enantiomers.

To a solution of the compound of Example 7b (96 mg, 0.3 mmol) in methanol (2 ml) was added dropwise hydrogen chloride in diethyl ether (1M, 0.3 ml). After stirring for 30 min, diethyl ether (10 ml) was added dropwise and the precipitate was collected by filtration. The resulting solid was washed with 20% methanol:diethyl ether (2×10 ml), followed by diethyl ether (2×10 ml) and dried in a vacuum oven at 50° C. to give the hydrochloride salt, the compound of Example 7c (98 mg), as a pair of enantiomers. HPLC Method A—retention time 10.24 min.

| Exam- | Structure | MH⁺ | MH⁺ | Bovine | Porcine |
|---|---|---|---|---|---|
| ple | Comment | found | expected | EC₅₀ nM | EC₅₀ nM |
| 7a | Mixture of 4 | 369.4 | 369.2 | 4.6 | 5.5 |
| | diastereoisomers | | | | |
| 7c | Second eluting pair | 369.2 | 369.2 | 3.8 | 5.5 |
| | of enantiomers - | | | | |
| | HPLC Method A - | | | | |
| | hydrochloride salt | | | | |

### Example 7a

¹H-NMR (CD₃OD): 1.10-1.19 (3H), 4.65-4.71 (1H), 6.10-6.22 (1H), 6.33-6.40 (1H), 7.00-7.12 (2H), 7.18-7.29 (1H), 7.40-7.52 (1H)

### Example 7c

¹H-NMR (CD₃OD): 1.40-1.44 (3H), 2.09-2.21 (2H), 4.95-4.99 (1H), 6.41-6.50 (2H), 7.01-7.04 (1H), 7.08-7.13 (1H), 7.29-7.32 (1H), 7.59-7.64 (1H)

### Example 8

### 7-Hydroxy-6-{[3-(1H-indol-3-yl)-1,3-dimethylbutyl]amino}-4,5,6,7-tetrahydroimidazo[4,5,1-jk][1]benzazepin-2(1H)-one

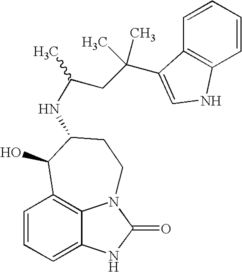

To a solution of the compound of Preparation 1 (232 mg, 0.9 mmol) in methanol (5 ml) was added triethylamine (0.3 ml, 1.9 mmol), followed by the compound of Preparation 53 (215 mg, 1.0 mmol) in methanol (5 ml). The reaction mixture was stirred at room temperature for 18 h, heated at reflux for 2 h and then stirred at room temperature for a further 60 h. To the mixture was added sodium borohydride (52 mg, 1.4 mmol) and the reaction mixture was stirred at room temperature for 14 days. The mixture was quenched with water, diluted with methanol and concentrated in vacuo. The residue was triturated with dichloromethane:methanol (1:9), filtered and concentrated in vacuo to give the crude product. The crude product was dissolved in acetonitrile:water (8:2, 4 ml) and purified by automated preparative liquid chromatography (Gilson system, 150×21.6 mm Gemini C18(2) 5 μm column, 20 ml/min) using an acetonitrile: 0.1% aqueous ammonia (90:10): acetonitrile: 0.1% aqueous ammonia (10:90) gradient [35:65 (for 25 min) to 95:5 (from 25 to 26 min) then at 95:5 (for 4 min)]. The appropriate fractions were combined and concentrated to give the compound of Example 8 (63 mg) as a pair of enantiomers. HPLC Method A—retention time 15.38 min.

Experimental MH⁺ 419.5; expected 419.2

¹H-NMR (CD₃OD): 0.96-1.00 (3H), 1.30-1.41 (6H), 4.25-4.28 (1H), 6.60-6.64 (1H), 6.77-6.86 (2H), 6.90-6.93 (3H), 7.14-7.16 (1H), 7.43-7.46 (1H)

Bovine EC₅₀—114 nM; Porcine EC₅₀—6.1 nM

### Example 9

### 7-Hydroxy-6-{[1-methyl-3-(1,3,5-trimethyl-1H-pyrazol-4-yl)propyl]amino}-4,5,6,7-tetrahydroimidazo[4,5,1-jk][1]benzazepin-2(1H)-one

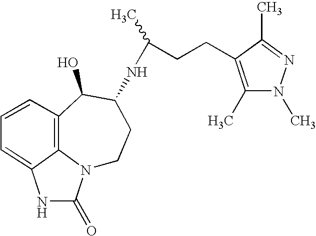

To a mixture of the compound of Preparation 1 (220 mg, 0.9 mmol) and the compound of Preparation 70 (189 mg, 1.1 mmol) in methanol (5 ml) was added triethylamine (36 ml, 0.3 mmol). After stirring for 1 h, sodium cyanoborohydride (81 mg, 1.3 mmol) was added and the reaction mixture was heated at 60° C. for 5 days. The mixture was concentrated in vacuo and to the residue was added dichloromethane (30 ml) and water (30 ml). The two layers were separated and the aqueous layer was extracted with dichloromethane (15 ml). The combined organic phases were washed with brine (20 ml), dried (MgSO₄) and concentrated in vacuo. The residue was dissolved in acetonitrile:water (9:1, 2 ml) and purified by automated preparative liquid chromatography (Gilson system, 150 mm×21.4 mm Gemini C18 5 μm column, 20 ml/min) using an acetonitrile:0.1% aqueous ammonia (5:95): acetonitrile:0.1% aqueous ammonia (95:5) gradient [90:10 to 80:20 (from 3 to 15 min) to 50:50 (from 20 to 25 min) to 5:95 (from 25 to 26 min)]. The appropriate fractions were combined and concentrated to give the compound of Example 9a (25 mg) as a pair of enantiomers. HPLC Method A—retention time 11.11 min. Other appropriate fractions were combined and concentrated to give the compound of Example 9b (36 mg) as a pair of enantiomers. HPLC Method A—retention time 11.34 min.

| Exam- | Structure | MH⁺ | MH⁺ | Bovine | Porcine |
|---|---|---|---|---|---|
| ple | Comment | found | expected | EC₅₀ nM | EC₅₀ nM |
| 9a | First eluting pair of | N/A | N/A | 402 | 268 |
| | enantiomers - | | | | |
| | HPLC Method A | | | | |
| 9b | Second eluting pair | N/A | N/A | 4.2 | 2.8 |
| | of enantiomers - | | | | |
| | HPLC Method A | | | | |

### Example 9a

¹H-NMR (CD₃OD): 1.17-1.20 (3H), 2.07-2.09 (3H), 2.11-2.13 (3H), 3.61-3.63 (3H), 4.66-4.68 (1H), 6.99-7.01 (1H), 7.02-7.05 (1H), 7.17-7.19 (1H)

### Example 9b

¹H-NMR (CD₃OD): 1.14-1.17 (3H), 2.09-2.11 (3H), 2.14-2.16 (3H), 3.62-3.64 (3H), 4.63-4.65 (1H), 6.99-7.01 (1H), 7.03-7.06 (1H), 7.18-7.20 (1H)

The following Examples were prepared by similar methods to those described above for Examples 1-9:

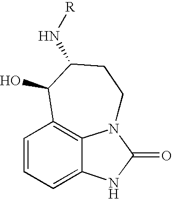

| | | | MH⁺ | EC₅₀ (nM) | From the |
|---|---|---|---|---|---|
| | | Structure | Found/ | Bovine/ | compound of |
| Example | R | Comment | Expected | Porcine | Preparation: |
| 10 | 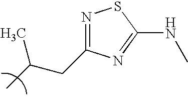 | Mixture of 4 diastereoisomers | 375.5 375.2 | 497 399 | 126 |
| 11 | 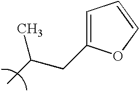 | Mixture of 4 diastereoisomers | 328.4 328.2 | 108 64 | 97 |
| 12 | 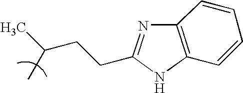 | Mixture of 4 diastereoisomers | 392.6 392.2 | 34 53 | 69 |
| 13 | 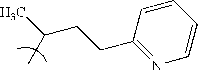 | Mixture of 4 diastereoisomers | 353.6 353.2 | 356 184 | 160 |
| 14a | 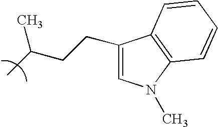 | Mixture of 4 diastereoisomers | 405.4 405.2 | 18 26 | 154 |
| 14b | 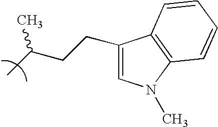 | Second eluting pair of enantiomers- HPLC method A | 405.6 405.2 | 8.0 5.8 | 154 |
| 14c | 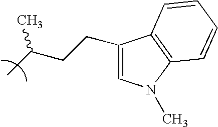 | Second eluting pair of enantiomers- HPLC method A- hydrochloride salt | 405.0 405.2 | 5.5 4.2 | 154 |
| 15 | 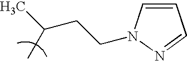 | Mixture of 4 diastereoisomers | 342.5 342.4 | 161 98 | 92 |
| 16 | 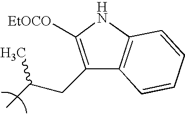 | Second eluting pair of enantiomers- HPLC method A | 449.5 449.2 | 26 10 | 159 |
| 17 | 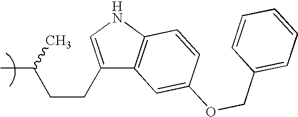 | Second eluting pair of enantiomers- HPLC method A | 497.5 497.3 | 25 33 | 158 |
| 18 | 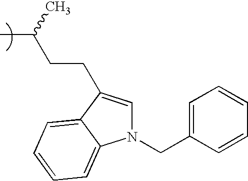 | Second eluting pair of enantiomers- HPLC method A | 481.5 481.3 | 218 98 | 157 |
| 19 | 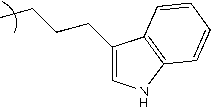 | Pair of enantiomers- HPLC method A | N/A | 50 65 | 153 |
| 20 | 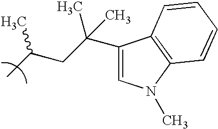 | Second eluting pair of enantiomers- HPLC method A | 433.4 433.3 | 157 36 | 52 |
| 21a | 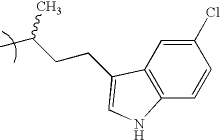 | First eluting pair of enantiomers- HPLC method A | 424.9 425.2 | 1830 <10000 | 42 |
| 21b | 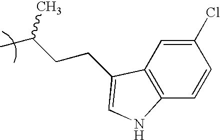 | Second eluting pair of enantiomers- HPLC method A | 424.9 425.2 | 15 11 | 42 |
| 22 | 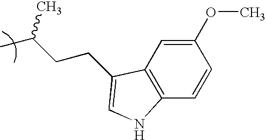 | Second eluting pair of enantiomers- HPLC method A | 421.4 421.2 | 50 53 | 44 |
| 23a | 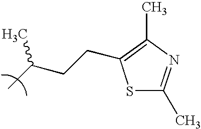 | First eluting pair of enantiomers- HPLC method A | N/A | 153 121 | 17 |
| 23b | 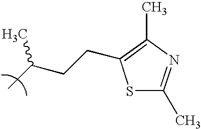 | Second eluting pair of enantiomers- HPLC method A | N/A | 12 4.9 | 17 |
| 24a | 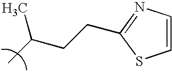 | Mixture of 4 diastereoisomers | 359.3 359.2 | 409 464 | 16 |
| 24b | 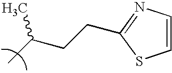 | Second eluting pair of enantiomers- HPLC method A | 359.4 359.2 | 92 79 | 16 |
| 25 | 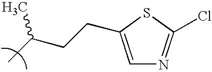 | Second eluting pair of enantiomers- HPLC method A | 393.3 393.1 | 2.9 4.3 | 116 |
| 26 | 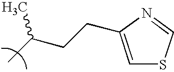 | Second eluting pair of enantiomers- HPLC method A | 359.4 359.2 | 345 742 | 18 |
| 27 | 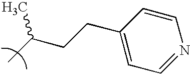 | Second eluting pair of enantiomers- HPLC method A | 353.4 353.2 | 77 53 | 22 |
| 28a | 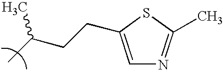 | First eluting pair of enantiomers- HPLC method A | 373.0 373.2 | 129 104 | 19 |
| 28b | 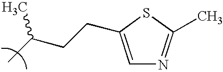 | Second eluting pair of enantiomers- HPLC method A | 373.4 373.2 | 6.6 <10000 | 19 |
| 29 | 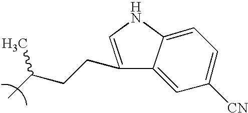 | Second eluting pair of enantiomers- HPLC method A | 416.4 416.2 | 83 127 | 45 |
| 30 | 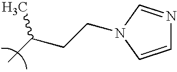 | Second eluting pair of enantiomers- HPLC method A | 342.4 342.2 | 48 3.8 | 161 |
| 31a | 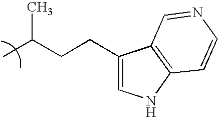 | Mixture of 4 diastereoisomers | 392.1 392.2 | 197 146 | 48 |
| 31b | 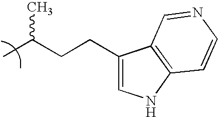 | First eluting pair of enantiomers- HPLC method A | 392.5 392.2 | 58 43 | 48 |
| 31c | 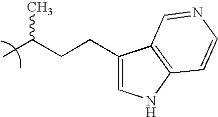 | Second eluting pair of enantiomers- HPLC method A | 392.5 392.2 | 366 19 | 48 |
| 32a | 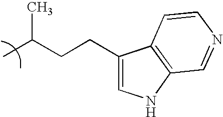 | Mixture of 4 diastereoisomers | 392.1 392.2 | 52 92 | 47 |
| 32b | 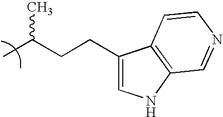 | First eluting pair of enantiomers- HPLC method A | 392.5 392.2 | 96 1090 | 47 |
| 32c | 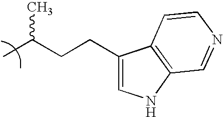 | Second eluting pair of enantiomers- HPLC method A | 392.5 392.2 | 18 8.7 | 47 |
| 33 | 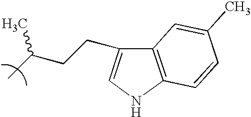 | Second eluting pair of enantiomers- HPLC method A | 405.5 405.2 | 74 24 | 46 |
| 34a | 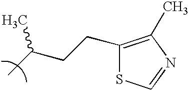 | First eluting pair of enantiomers- HPLC method A | 373.4 373.2 | 23 45 | 21 |
| 34b | 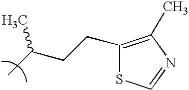 | Second eluting pair of enantiomers- HPLC method A | 373.4 373.2 | 1.4 2.1 | 21 |
| 35 | 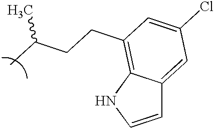 | Second eluting pair of enantiomers- HPLC method A | N/A | 0.8 2.0 | 20 |
| 36 | 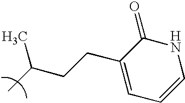 | Mixture of 4 diastereoisomers | 369.5 369.2 | 59 56 | 23 |
| 37 | 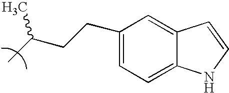 | Second eluting pair of enantiomers- HPLC method A | 391.1 391.2 | 16 28 | 24 |

### Example 10

### 7-Hydroxy-6-({1-methyl-2-[5-(methylamino)-1,2,4-thiadiazol-3-yl]ethyl}amino)-4,5,6,7-tetrahydroimidazo[4,5,1-jk][1]benzazepin-2(1H)-one

¹H-NMR (CD₃OD): 1.11-1.18 (3H), 2.39-2.44 (2H), 2.83-2.84 (3H), 4.70-4.76 (1H), 6.99-7.08 (2H), 7.17-7.19 (1H)

### Example 11

### 6-{[2-(2-Furyl)-1-methylethyl]amino}-7-hydroxy-4,5,6,7-tetrahydroimidazo[4,5,1-jk][1]benzazepin-2(1H)-one

¹H-NMR (CD₃OD): 1.05-1.11 (3H), 2.61-2.74 (2H), 4.61-4.70 (1H), 6.00-6.06 (1H), 6.22-6.24 (1H), 6.99-7.19 (3H), 7.32-7.34 (1H)

### Example 12

### 6-{[3-(1H-Benzimidazol-2-yl)-1-methylpropyl]amino}-7-hydroxy-4,5,6,7-tetrahydroimidazo[4,5,1-jk][1]benzazepin-2(1H)-one

¹H-NMR (CD₃OD): 1.16-1.22 (3H), 1.70-1.84 (2H), 4.62-4.68 (1H), 6.97-7.08 (2H), 7.16-7.21 (3H), 7.40-7.45 (2H)

### Example 13

### 7-Hydroxy-6-[(1-methyl-3-pyridin-2-ylpropyl)amino]-4,5,6,7-tetrahydroimidazo[4,5,1-jk][1]benzazepin-2(1H)-one

¹H-NMR (CD₃OD): 1.10-1.20 (3H), 1.68-1.81 (2H), 4.65-4.68 (1H), 6.99-7.07 (2H), 7.18-7.31 (3H), 7.70-7.75 (1H), 8.38-8.41 (1H)

### Example 14a

### 7-Hydroxy-6-{[1-methyl-3-(1-methyl-1H-indol-3-yl)propyl]amino}-4,5,6,7-tetrahydroimidazo[4,5,1-jk][1]benzazepin-2(1H)-one

¹H-NMR (CD₃OD): 1.02-1.10 (3H), 3.67-3.70 (3H), 4.50-4.54 (1H), 6.83-7.00 (4H), 7.02-7.12 (2H), 7.31-7.35 (1H), 7.43-7.49 (1H)

### Example 14b

### 7-Hydroxy-6-{[1-methyl-3-(1-methyl-1H-indol-3-yl)propyl]amino}-4,5,6,7-tetrahydroimidazo[4,5,1-jk][1]benzazepin-2(1H)-one

¹H-NMR (CD₃OD): 1.10-1.20 (3H), 3.64-3.70 (3H), 4.59-4.65 (1H), 6.85-6.92 (1H), 6.94-7.10 (3H), 7.10-7.20 (2H), 7.45-7.51 (1H)

### Example 14c

### 7-Hydroxy-6-{[1-methyl-3-(1-methyl-1H-indol-3-yl)propyl]amino}-4,5,6,7-tetrahydroimidazo[4,5,1-jk][1]benzazepin-2(1H)-one

No n.m.r. data available

### Example 15

### 7-Hydroxy-6-{[1-methyl-3-(1H-pyrazol-1-yl)propyl]amino}-4,5,6,7-tetrahydroimidazo[4,5,1-jk][1]benzazepin-2(1H)-one

¹H-NMR (CD₃OD): 1.05-1.15 (3H), 4.62-4.69 (1H), 6.19-7.25 (1H), 6.98-7.10 (2H), 7.19-7.24 (1H), 7.40-7.46 (1H), 7.59-7.61 (1H)

### Example 16

### Ethyl 3-(2-{[7-hydroxy-2-oxo-1,2,4,5,6,7-hexahydroimidazo[4,5,1-jk][1]benzazepin-6-yl]amino}propyl)-1H-indole-2-carboxylate

¹H-NMR (d₆-DMSO): 0.85-0.94 (3H), 1.15-1.23 (3H), 4.20-4.31 (2H), 4.52-4.56 (1H), 6.80-6.87 (2H), 6.95-7.02 (2H), 7.19-7.23 (1H), 7.36-7.40 (1H), 7.59-7.63 (1H)

### Example 17

### 6-([3-[5-(Benzyloxy)-1H-indol-3-yl]-1-methylpropyl]amino)-7-hydroxy-4,5,6,7-tetrahydroimidazo[4,5,1-jk][1]benzazepin-2(1H)-one

¹H-NMR (CD₃OD): 1.14-1.17 (3H), 2.70-2.81 (2H), 4.60-4.62 (1H), 5.05-5.06 (2H), 6.80-6.83 (1H), 6.98-7.02 (2H), 7.04-7.06 (2H), 7.17-7.20 (2H), 7.22-7.24 (1H), 7.30-7.36 (2H), 7.42-7.45 (2H)

### Example 18

### 6-{[3-(1-Benzyl-1H-indol-3-yl)-1-methylpropyl]amino}-7-hydroxy-4,5,6,7-tetrahydroimidazo[4,5,1-jk][1]benzazepin-2(1H)-one

¹H-NMR (CD₃OD): 1.12-1.16 (3H), 1.75-1.95 (2H), 4.59-4.62 (1H), 5.25-5.27 (2H), 6.98-7.03 (4H), 7.05-7.11 (4H), 7.16-7.24 (4H), 7.51-7.53 (1H)

### Example 19

### 7-Hydroxy-6-{[3-(1H-indol-3-yl)propyl]amino}-4,5,6,7-tetrahydroimidazo[4,5,1-jk][1]benzazepin-2(1H)-one

¹H-NMR (CD₃OD): 4.65-4.69 (1H), 6.96-7.07 (5H), 7.18-7.21 (1H), 7.29-7.32 (1H), 7.50-7.54 (1H)

### Example 20

### 6-{[3-Dimethyl-3-(1-methyl-1H-indol-3-yl)butyl]amino}-7-hydroxy-4,5,6,7-tetrahydroimidazo[4,5,1-jk][1]benzazepin-2(1H)-one

¹H-NMR (CD₃OD): 0.96-1.00 (3H), 1.25-1.39 (6H), 3.66-3.68 (3H), 4.35-4.38 (1H), 6.59-6.61 (1H), 6.78-6.82 (3H), 6.90-6.92 (2H), 7.02-7.05 (1H), 7.42-7.44 (1H)

### Example 21a

### 6-{[3-(5-Chloro-1H-indol-3-yl)-1-methylpropyl]amino}-7-hydroxy-4,5,6,7-tetrahydroimidazo[4,5,1-jk][1]benzazepin-2(1H)-one

¹H-NMR (CD₃OD): 1.24-1.29 (3H), 1.68-1.79 (2H), 4.61-4.65 (1H), 6.95-7.05 (4H), 7.11-7.15 (1H), 7.22-7.27 (1H), 7.42-7.45 (1H)

### Example 21b

### 6-{[3-(5-Chloro-1H-indol-3-yl)-1-methylpropyl]amino}-7-hydroxy-4,5,6,7-tetrahydroimidazo[4,5,1-jk][1]benzazepin-2(1H)-one

¹H-NMR (CD₃OD): 1.12-1.17 (3H), 1.69-1.79 (2H), 4.59-4.63 (1H), 6.95-7.05 (4H), 7.14-7.18 (1H), 7.22-7.25 (1H), 7.44-7.47 (1H)

### Example 22

### 7-Hydroxy-6-{[3-(5-methoxy-1H-indol-3-yl)-1-methylpropyl]amino}-4,5,6,7-tetrahydroimidazo[4,5,1-jk][1]benzazepin-2(1H)-one

¹H-NMR (CD₃OD): 1.15-1.18 (3H), 3.80-3.81 (3H), 4.60-4.63 (1H), 6.70-6.73 (1H), 6.98-7.05 (3H), 7.15-7.20 (2H)

### Example 23a

### 6-{[3-(2,4-Dimethyl-1,3-thiazol-5-yl)-1-methylpropyl]amino}-7-hydroxy-4,5,6,7-tetrahydroimidazo[4,5,1-jk][1]benzazepin-2(1H)-one

¹H-NMR (CD₃OD): 1.20-1.24 (3H), 2.19-2.22 (3H), 2.54-2.58 (3H), 4.70-4.74 (1H), 6.98-7.01 (1H), 7.02-7.07 (1H), 7.16-7.20 (1H)

### Example 23b

### 6-{[3-(2,4-Dimethyl-1,3-thiazol-5-yl)-1-methylpropyl]amino}-7-hydroxy-4,5,6,7-tetrahydroimidazo[4,5,1-jk][1]benzazepin-2(1H)-one

¹H-NMR (CD₃OD): 1.13-1.16 (3H), 2.22-2.25 (3H), 2.56-2.59 (3H), 4.65-4.68 (1H), 6.98-7.02 (1H), 7.04-7.09 (1H), 7.19-7.23 (1H)

### Example 24a 7-Hydroxy-6-{[1-methyl-3-(1,3-thiazol-2-yl)propyl]amino}-4,5,6,7-tetrahydroimidazo[4,5,1-jk][1]benzazepin-2(1H)-one

¹H-NMR (CD₃OD): 1.15-1.22 (3H), 1.76-1.91 (2H), 4.67-4.71 (1H), 6.98-7.08 (2H), 7.17-7.23 (1H), 7.40-7.43 (1H), 7.60-7.64 (1H)

### Example 24b

### 7-Hydroxy-6-{[1-methyl-3-(1,3-thiazol-2-yl)propyl]amino}-4,5,6,7-tetrahydroimidazo[4,5,1-jk][1]benzazepin-2(1H)-one

¹H-NMR (CD₃OD): 1.14-1.17 (3H), 1.82-1.96 (2H), 4.63-4.66 (1H), 6.99-7.08 (2H), 7.20-7.22 (1H), 7.40-7.41 (1H), 7.60-7.62 (1H)

### Example 25

### 6-{[3-(2-Chloro-1,3-thiazol-5-yl)-1-methylpropyl]amino}-7-hydroxy-4,5,6,7-tetrahydroimidazo[4,5,1-jk][1]benzazepin-2(1H)-one

¹H-NMR (CD₃OD): 1.20-1.26 (3H), 4.75-4.78 (1H), 7.00-7.02 (1H), 7.04-7.09 (1H), 7.24-7.27 (1H), 7.36-7.38 (1H)

### Example 26

### 7-Hydroxy-6-{[1-methyl-3-(1,3-thiazol-4-yl)propyl]amino}-4,5,6,7-tetrahydroimidazo[4,5,1-jk][1]benzazepin-2(1H)-one

¹H-NMR (CD₃OD): 1.20-1.25 (3H), 1.71-1.85 (2H), 4.71-4.74 (1H), 7.00-7.09 (2H), 7.19-7.25 (2H), 8.91-8.93 (1H)

### Example 27

### 7-Hydroxy-6-[(1-methyl-3-pyridin-4-ylpropyl)amino]-4,5,6,7-tetrahydroimidazo[4,5,1-jk][1]benzazepin-2(1H)-one

¹H-NMR (CD₃OD): 1.16-1.20 (3H), 1.78-1.86 (2H), 4.63-4.66 (1H), 6.99-7.08 (2H), 7.20-7.23 (1H), 7.30-7.33 (2H), 8.38-8.41 (2H)

### Example 28a

### 7-Hydroxy-6-{[(1-methyl-3-(2-methyl-1,3-thiazol-5-yl)propyl]amino}-4,5,6,7-tetrahydroimidazo[4,5,1-jk][1]benzazepin-2(1H)-one

¹H-NMR (CD₃OD): 1.15-1.18 (3H), 1.75-1.84 (2H), 4.64-4.66 (1H), 7.00-7.08 (2H), 7.17-7.19 (1H), 7.24-7.25 (1H)

### Example 28b

### 7-Hydroxy-6-{[1-methyl-3-(2-methyl-1,3-thiazol-5-yl)propyl]amino}-4,5,6,7-tetrahydroimidazo[4,5,1-jk][1]benzazepin-2(1H)-one

¹H-NMR (CD₃OD): 1.11-1.14 (3H), 1.78-1.85 (2H), 4.63-4.65 (1H), 6.99-7.07 (2H), 7.20-7.22 (1H), 7.30-7.31 (1H)

### Example 29

### 3-(3-{[7-Hydroxy-2-oxo-1,2,4,5,6,7-hexahydroimidazo[4,5,1-jk][1]benzazepin-6-yl]amino}butyl)-1H-indole-5-carbonitrile

¹H-NMR (CD₃OD): 1.18-1.21 (3H), 1.72-1.81 (2H), 4.61-4.63 (1H), 6.99-7.06 (2H), 7.19-7.20 (1H), 7.35-7.37 (1H), 7.42-7.44 (1H), 7.98-7.99 (1H)

### Example 30

### 7-Hydroxy-6-{[3-(1H-imidazol-1-yl)-1-methylpropyl]amino}-4,5,6,7-tetrahydroimidazo[4,5,1-jk][1]benzazepin-2(1H)-one

¹H-NMR (CD₃OD): 1.10-1.13 (3H), 1.85-1.91 (2H), 4.62-4.64 (1H), 6.96-6.98 (1H), 7.00-7.09 (2H), 7.11-7.13 (1H), 7.21-7.23 (1H), 7.61-7.62 (1H)

### Example 31a

### 7-Hydroxy-6-{[1-methyl-3-(1H-pyrrolo[3,2-c]pyridin-3-yl)propyl]amino}-4,5,6,7-tetrahydroimidazo[4,5,1-jk][1]benzazepin-2(1H)-one

¹H-NMR (CD₃OD): 1.09-1.19 (3H), 1.89-1.98 (2H), 4.58-4.63 (1H), 6.59-6.62 (1H), 6.96-7.06 (3H), 7.37-7.41 (1H), 8.01-8.04 (1H), 8.70-8.72 (1H)

### Example 31b

### 7-Hydroxy-6-{[1-methyl-3-(1H-pyrrolo[3,2-c]pyridin-3-yl)propyl]amino}-4,5,6,7-tetrahydroimidazo[4,5,1-jk][1]benzazepin-2(1H)-one

¹H-NMR (CD₃OD): 1.15-1.17 (3H), 1.90-1.96 (2H), 4.60-4.62 (1H), 6.60-6.62 (1H), 7.00-7.04 (2H), 7.18-7.20 (1H), 7.37-7.39 (1H), 7.40-7.42 (1H), 8.08-8.10 (1H)

### Example 31c

### 7-Hydroxy-6-{[1-methyl-3-(1H-pyrrolo[3,2-c]pyridin-3-yl)propyl]amino}-4,5,6,7-tetrahydroimidazo[4,5,1-jk][1]benzazepin-2(1H)-one

¹H-NMR (CD₃OD): 1.13-1.16 (3H), 1.91-1.97 (2H), 4.60-4.62 (1H), 6.61-6.62 (1H), 6.99-7.01 (1H), 7.03-7.05 (1H), 7.18-7.20 (1H), 7.37-7.39 (1H), 7.40-7.42 (1H), 8.08-8.10 (1H)

### Example 32a

### 7-Hydroxy-6-{[1-methyl-3-(1H-pyrrolo[2,3-c]pyridin-3-yl)propyl]amino}-4,5,6,7-tetrahydroimidazo[4,5,1-jk][1]benzazepin-2(1H)-one

¹H-NMR (CD₃OD): 1.10-1.25 (3H), 1.95-2.10 (2H), 4.61-4.64 (1H), 6.55-6.58 (1H), 6.98-7.20 (2H), 7.48-7.61 (2H), 8.01-8.04 (1H), 8.70-8.81 (1H)

### Example 32b

### 7-Hydroxy-6-{[1-methyl-3-(1H-pyrrolo[2,3-c]pyridin-3-yl)propyl]amino}-4,5,6,7-tetrahydroimidazo[4,5,1-jk][1]benzazepin-2(1H)-one

No n.m.r. data available

### Example 32c

### 7-Hydroxy-6-{[(1-methyl-3-(1H-pyrrolo[2,3-c]pyridin-3-yl)propyl]amino}-4,5,6,7-tetrahydroimidazo[4,5,1-jk][1]benzazepin-2(1H)-one

¹H-NMR (CD₃OD): 1.10-1.13 (3H), 1.95-2.00 (2H), 4.60-4.63 (1H), 6.51-6.53 (1H), 6.99-7.06 (2H), 7.19-7.21 (1H), 7.50-7.52 (1H), 7.55-7.57 (1H), 8.02-8.04 (1H)

### Example 33

### 7-Hydroxy-6-{[(1-methyl-3-(5-methyl-1H-indol-3-yl)propyl]amino}-4,5,6,7-tetrahydroimidazo[4,5,1-jk][1]benzazepin-2(1H)-one

¹H-NMR (CD₃OD): 1.15-1.18 (3H), 1.65-1.80 (2H), 1.38-1.39 (3H), 4.60-4.63 (1H), 6.89-6.96 (2H), 7.00-7.09 (2H), 7.15-7.19 (2H), 7.17-7.18 (1H)

### Example 34a

### 7-Hydroxy-6-{[(1-methyl-3-(4-methyl-1,3-thiazol-5-yl)propyl]amino}-4,5,6,7-tetrahydroimidazo[4,5,1-jk][1]benzazepin-2(1H)-one

¹H-NMR (CD₃OD): 1.18-2.00 (3H), 2.28-2.29 (3H), 4.64-4.66 (1H), 6.99-7.06 (2H), 7.17-7.19 (1H), 8.65-8.67 (1H)

### Example 34b

### 7-Hydroxy-6-{[(1-methyl-3-(4-methyl-1,3-thiazol-5-yl)propyl]amino}-4,5,6,7-tetrahydroimidazo[4,5,1-jk][1]benzazepin-2(1H)-one

¹H-NMR (CD₃OD): 1.15-1.18 (3H), 2.36-2.38 (3H), 4.62-4.64 (1H), 699-7.07 (2H), 7.20-7.23 (1H), 8.70-8.71 (1H)

### Example 35

### 6-{[3-(5-Chloro-1H-indol-7-yl)-1-methylpropyl]amino}-7-hydroxy-4,5,6,7-tetrahydroimidazo[4,5,1-jk][1]benzazepin-2(1H)-one

¹H-NMR (CD₃OD): 1.17-1.20 (3H), 4.63-4.66 (1H), 6.36-6.38 (1H), 6.89-6.90 (1H), 7.00-7.03 (1H), 7.05-7.10 (1H), 7.18-7.23 (2H), 7.32-7.33 (1H)

### Example 36

### 7-Hydroxy-6-{[1-methyl-3-(2-oxo-1,2-dihydropyridin-3-yl)propyl]amino}-4,5,6,7-tetrahydroimidazo[4,5,1-jk][1]benzazepin-2(1H)-one

¹H-NMR (CD₃OD): 1.15-1.19 (3H), 1.72-1.85 (2H), 4.62-4.64 (1H), 6.29-6.35 (1H), 6.98-7.09 (2H), 7.20-7.30 (2H), 7.41-7.46 (1H)

### Example 37

### 7-Hydroxy-6-{[3-(1H-indol-5-yl)-1-methylpropyl]amino}-4,5,6,7-tetrahydroimidazo[4,5,1-jk][1]benzazepin-2(1H)-one

¹H-NMR (CD₃OD): 1.10-1.13 (3H), 4.59-4.61 (1H), 6.31-6.33 (1H), 6.89-6.91 (1H), 6.98-7.02 (2H), 7.16-7.18 (2H), 7.21-7.23 (1H), 7.27-7.29 (1H)

### Example 38

### 7-Hydroxy-6-{[3-(1H-indol-7-yl)-1-methylpropyl]amino}-4,5,6,7-tetrahydroimidazo[4,5,1-jk][1]benzazepin-2(1H)-one

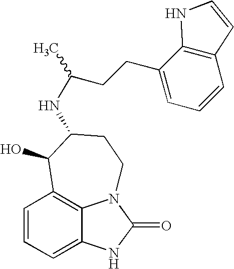

To a mixture of the compound of Preparation 1 (1.2 g, 4.6 mmol) and the compound of Preparation 33 (850 mg, 4.6 mmol) in methanol (45 ml), at 0° C., was added triethylamine (0.4 ml, 2.8 mmol). After stirring for 1 h, sodium cyanoborohydride (721 mg, 11.5 mmol) was added and the reaction mixture was stirred at room temperature for 60 h and then at 60° C. for 18 h. The mixture was quenched with water (1 ml) and concentrated in vacuo. The residue was azeotroped with methanol and then pre-absorbed on to silica (5 g). The silica/product mix was purified by automated flash chromatography (Biotage™ 65i cartridge conditioned with dichloromethane:2% methanolic ammonia with gradient elution, dichloromethane:2% methanolic ammonia [98:2 to 90:10]. The appropriate fractions were combined and concentrated to give the compound of Example 38a (331 mg) as a pair of enantiomers. HPLC Method A—retention time 14.67 min. Other appropriate fractions were combined and concentrated to give the compound of Example 38b (167 mg) as a pair of enantiomers. HPLC Method A—retention time 14.93 min.

To a solution of the compound of Example 38a (330 mg, 0.9 mmol) in methanol (5 ml), at 0° C., was added dropwise hydrogen chloride in diethyl ether (1M, 0.9 ml). After stirring for 1 h, diethyl ether (25 ml) was added dropwise and the precipitate was collected by filtration. The resulting solid was washed with 15% methanol/diethyl ether (25 ml), followed by diethyl ether (3×15 ml), and dried in a vacuum oven at 50° C. The solid was re-crystallised from hot isopropanol:water and washed with cold isopropanol (3×5 ml) and diethyl ether (3×15 ml), before drying in a vacuum oven at 50° C. to give the hydrochloride salt, the compound of Example 38c (84 mg), as a pair of enantiomers. HPLC Method A—retention time 14.69 min.

To a solution of the compound of Example 38b (63 mg, 0.2 mmol) in methanol (1 ml), at 0° C., was added dropwise hydrogen chloride in diethyl ether (1M, 0.2 ml). After stirring for 30 min, diethyl ether (5 ml) was added dropwise and the precipitate was collected by filtration. The resulting solid was washed with diethyl ether (2×5 ml), and dried in a vacuum oven at 50° C. to give the hydrochloride salt, the compound of Example 38d (69 mg), as a pair of enantiomers. HPLC Method A—retention time 14.69 min.

| Exam- | Structure | MH⁺ | MH⁺ | Bovine | Porcine |
|---|---|---|---|---|---|
| ple | Comment | found | expected | EC₅₀ nM | EC₅₀ nM |
| 38a | First eluting pair of | 391.3 | 391.2 | 44 | 79 |
| | enantiomers - | | | | |
| | HPLC method A | | | | |
| 38b | Second eluting pair | 391.3 | 391.2 | 0.8 | 0.9 |
| | of enantiomers - | | | | |
| | HPLC method A | | | | |
| 38c | First eluting pair of | 391.3 | 391.2 | 137 | 174 |
| | enantiomers - | | | | |
| | HPLC method A - | | | | |
| | hydrochloride salt | | | | |
| 38d | Second eluting pair | 391.1 | 391.2 | 0.6 | 1.1 |
| | of enantiomers - | | | | |
| | HPLC method A - | | | | |
| | hydrochloride salt | | | | |

### Example 38a

¹H-NMR (CD₃OD): 1.19-1.24 (3H), 1.62-1.78 (2H), 4.61-4.64 (1H), 6.35-6.37 (1H), 6.79-6.87 (2H), 6.94-7.04 (2H), 7.08-7.14 (2H), 7.32-7.36 (1H)

### Example 38b

¹H-NMR (CD₃OD): 1.13-1.18 (3H), 1.76-1.93 (2H), 4.59-4.62 (1H), 6.35-6.38 (2H), 6.83-6.86 (1H), 6.97-7.00 (1H), 7.02-7.06 (1H), 7.10-7.12 (1H), 7.14-7.19 (1H), 7.29-7.34 (1H)

### Example 38c

¹H-NMR (CD₃OD): 1.43-1.48 (3H), 1.90-2.03 (2H), 4.79-4.81 (1H), 6.37-6.39 (1H), 6.91-6.93 (2H), 7.00-7.02 (1H), 7.04-7.09 (2H), 7.20-7.22 (1H), 7.39-7.42 (1H)

### Example 38d

¹H-NMR (CD₃OD): 1.43-1.47 (3H), 1.94-2.12 (2H), 4.85-4.89 (1H), 6.40-6.43 (1H), 6.92-6.96 (2H), 7.00-7.03 (1H), 7.06-7.10 (1H), 7.21-7.26 (2H), 7.37-7.41 (1H)

The following was prepared analogously:

### Example 39

### 7-Hydroxy-6-{[3-(3-hydroxypyridin-2-yl)-1-methylpropyl]amino}-4,5,6,7-tetrahydroimidazo[4,5,1-jk][1]benzazepin-2(1H)-one

From the compound of Preparation 32, as a pair of enantiomers.

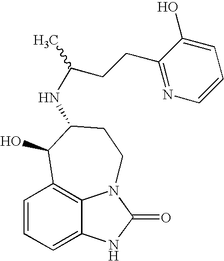

HPLC Method A—retention time 8.35 min.

Experimental MH⁺ 369.5; expected 369.2

¹H-NMR (CD₃OD): 1.17-1.19 (3H), 4.70-4.72 (1H), 6.99-7.01 (1H), 7.03-7.09 (2H), 7.16-7.18 (1H), 7.22-7.24 (1H), 7.81-7.83 (1H)

Bovine EC₅₀—330 nM; Porcine EC₅₀—159 nM

### Example 40

### 7-Hydroxy-6-{[2-(1H-indol-3-yl)ethyl]amino}-4,5,6,7-tetrahydroimidazo[4,5,1-jk][1]benzazepin-2(1H)-one

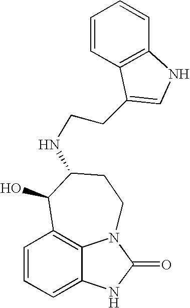

To a mixture of the compound of Preparation 1 (2.0 g, 7.8 mmol) and Preparation 122 (1.8 g, 7.8 mmol) in N,N-dimethyl formamide (20 ml) was added sodium carbonate (2.5 g, 23.5 mmol) and the reaction mixture was stirred at 50° C. for 18 h. The mixture was concentrated in vacuo and the residue was azeotroped with methanol. To the residue was added dichloromethane (20 ml) and methanol (4 ml) and the solid material was removed by filtration. The solution was concentrated in vacuo and the residue was dissolved in dichloromethane (20 ml) and methanol (2 ml) and purified by automated flash chromatography (Biotage™ 65i cartridge conditioned with dichloromethane:2% methanolic ammonia with gradient elution, dichloromethane:2% methanolic ammonia [98:2 to 80:20]. The appropriate fractions were combined and concentrated to give the compound of Example 40a (1.1 g) as a racemic mixture.

To a solution of the compound of Example 40a (1.1 g, 3.1 mmol) in methanol (15 ml), at 0° C., was added dropwise hydrogen chloride in diethyl ether (1M, 3.1 ml). After stirring at 0° C. for 30 min, diethyl ether (85 ml) was added dropwise and the precipitate was collected by filtration. The resulting solid was washed with 15% methanol/diethyl ether (30 ml), followed by diethyl ether (2×30 ml), and dried in a vacuum oven at 50° C. to give the hydrochloride salt, the compound of Example 40b (1.1 g) as a racemic mixture.

| Exam- | Structure | MH⁺ | MH⁺ | Bovine | Porcine |
|---|---|---|---|---|---|
| ple | Comment | found | expected | EC₅₀ nM | EC₅₀ nM |
| 40a | Racemic mixture | 363.4 | 363.2 | 459 | 20 |
| 40b | Racemic mixture - | 363.3 | 363.2 | 550 | 28 |
| | hydrochloride salt | | | | |

### Example 40a

¹H-NMR (CD₃OD): 1.79-1.89 (1H), 2.34-2.43 (1H), 2.93-3.17 (5H), 3.77-3.85 (1H), 3.94-4.02 (1H), 4.64-4.68 (1H), 6.94-7.02 (3H), 7.04-7.10 (3H), 7.31-7.34 (1H), 7.52-7.55 (1H)

### Example 40b

¹H-NMR (CD₃OD): 2.03-2.14 (1H), 2.46-2.55 (1H), 3.19-3.23 (2H), 3.38-3.61 (3H), 3.70-3.78 (1H), 4.15-4.22 (1H), 4.95-4.98 (1H), 6.99-7.12 (4H), 7.19-7.21 (1H), 7.25-7.28 (1H). 7.33-7.36 (1H), 7.56-7.60 (1H)

The following was prepared analogously:

### Example 41

### Ethyl-6-chloro-3-(2-{[7-hydroxy-2-oxo-1,2,4,5,6,7-hexahydroimidazo[4,5,1-jk][1]benzazepin-6-yl]amino}ethyl)-1H-indole-2-carboxylate

From the compound of Preparation 162, as a racemic mixture.

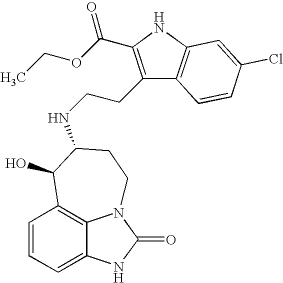

Experimental MH⁺ 469.5; expected 469.2

¹H-NMR (CD₃OD): 1.39-1.42 (3H), 4.35-4.41 (2H), 4.64-4.67 (1H), 6.97-7.03 (3H), 7.05-7.07 (1H), 7.39-7.41 (1H), 7.60-7.63 (1H)

Bovine EC₅₀—546 nM; Porcine EC₅₀—26 nM

### Example 42

### 6-{[3-(1H-Benzimidazol-5-yl)-1-methylprop-2-en-1-yl]amino}-7-hydroxy-4,5,6,7-tetrahydroimidazo[4,5,1-jk][1]benzazepin-2(1H)-one

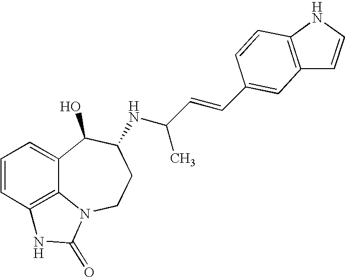

To a solution of the compound of Preparation 155 (1.0 g, 6.8 mmol) in acetone (4 ml), at 0° C., was added dropwise aqueous sodium hydroxide solution (4 ml) and the reaction mixture was allowed to warm to room temperature and stirred for 2 h. The mixture was adjusted to pH 7 by addition of concentrated hydrochloric acid and extracted with ethyl acetate. The combined extracts were dried (MgSO₄) and concentrated in vacuo to give 4-(1H-benzimidazol-6-yl)but-3-en-2-one (1.3 g) which was used directly.

A mixture of the compound of Preparation 1 (200 mg, 0.9 mmol) and 4-(1H-benzimidazol-6-yl)but-3-en-2-one (339 mg, 1.8 mmol) in methanol (10 ml) was stirred at room temperature for 18 h. Sodium borohydride (104 mg, 2.7 mmol) was added carefully and the reaction mixture was stirred at room temperature for 1 h. The mixture was diluted with methanol (8 ml) and Amberlyst® 15 ion-exchange resin (3.5 g, prepared according to J. Org. Chem. 1998, 63, 3471-3473) was added. The mixture was shaken overnight and the solution was filtered off. The resin was washed with methanol (5×20 ml) and treated with ammonia in methanol (2N, 15 ml) to release the captured product. After shaking for 2 h, the solution was filtered off and the resin was washed with ammonia in methanol (2N, 2×15 ml). The combined methanol/ammonia washings were concentrated in vacuo and the residue was dissolved in acetonitrile:water (1:1,1 ml) and purified by automated preparative liquid chromatography (Gilson system, 150 mm×21.4 mm Gemini 5 μm column, 20 ml/min) using an acetonitrile: 0.1% aqueous ammonia (1:9): acetonitrile:0.1% aqueous ammonia (9:1) gradient [1:0 to 2:8 (from 2 to 20 min) to 0:1 (from 20 to 21 min) then at 0:1 (for 4 min)]. The appropriate fractions were combined and concentrated to give the compound of Example 42 (3 mg) as a mixture of 4 diastereoisomers.

Experimental MH⁺ 390.5; expected 390.2

Bovine EC₅₀—23 nM; Porcine EC₅₀—22 nM

### Example 43

### 6-{[3-(1-Benzofuran-5-yl)-1-methylpropyl]amino}-7-hydroxy-4,5,6,7-tetrahydroimidazo[4,5,1-jk][1]benzazepin-2(1H)-one

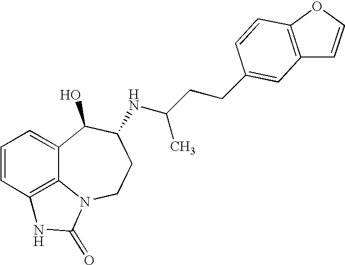

To a solution of the compound of Preparation 120 (1.0 g, 6.8 mmol) in acetone (4 ml), at 0° C., was added dropwise aqueous sodium hydroxide solution (4 ml) and the reaction mixture was allowed to warm to room temperature and stirred for 2 h. The mixture was adjusted to pH 7 by addition of concentrated hydrochloric acid and extracted with ethyl acetate. The combined extracts were dried (MgSO₄) and concentrated in vacuo to give 4-(1-benzofuran-5-yl)but-3-en-2-one (1.2 g) which was used directly.

A mixture of the compound of Preparation 1 (100 mg, 0.4 mmol), triethylamine (0.2 ml, 1.2 mmol) and 4-(1-benzofuran-5-yl)but-3-en-2-one (146 mg, 0.8 mmol) in methanol (3 ml) was stirred at room temperature for 18 h. Sodium borohydride (44 mg, 1.2 mmol) was then added and the reaction mixture was stirred at room temperature for 1 h. The mixture was diluted with methanol (8 ml) and Amberlyst® 15 ion-exchange resin (4 g, prepared according to J. Org. Chem. 1998, 63, 3471-3473) was added. The mixture was shaken overnight and the solution was filtered off. The resin was washed with methanol (3×20 ml) and treated with ammonia in methanol (2N, 15 ml). After shaking for 2 h, the solution was filtered off and the resin was washed with ammonia in methanol (2N, 2×15 ml). The combined methanolic ammonia washings were concentrated in vacuo and the residue was re-dissolved in methanol (5 ml). This solution was filtered and the filtrate was concentrated in vacuo. The residue was dissolved in acetonitrile:water (1:1, 1.5 ml) and purified by automated preparative liquid chromatography (Gilson system, 150 mm×21.4 mm Gemini 5 μm column, 20 ml/min) using an acetonitrile:0.1% aqueous ammonia (1:9): acetonitrile: 0.1% aqueous ammonia (9:1) gradient [1:0 to 2:8 (from 2 to 20 min) to 0:1 (from 20 to 21 min) then at 0:1 (for 4 min)]. The appropriate fractions were combined and concentrated in vacuo.

A solution of the residue (20 mg, 51 μmol) and platinum dioxide (10 mol %, 1 mg) in methanol (1 ml) was shaken under hydrogen (60 psi) for 30 min. The mixture was filtered through Arbocel®, washing through with methanol, and the filtrate was concentrated in vacuo to give the compound of Example 43 (20 mg) as a mixture of 4 diastereoisomers.

Experimental MH⁺ 392.4; expected 392.2

¹H-NMR (CD₃OD): 1.11-1.20 (3H), 4.62-4.66 (1H), 6.69-6.81 (1H), 6.98-7.20 (4H), 7.35-7.42 (2H), 7.65-7.70 (1H)

Bovine EC₅₀—16 nM; Porcine EC₅₀—11 nM

### Example 44

### (6R,7R)-6-{[(1R)-3-(2-Aminopyridin-3-yl)-1-methylpropyl]amino}-7-hydroxy-4,5,6,7-tetrahydroimidazo[4,5,1-jk][1]benzazepin-2(1H)-one

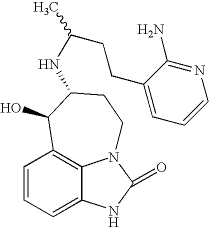

A mixture of the compound of Preparation 77 (566 mg, 1.4 mmol) and hydroxylamine hydrochloride (486 mg, 7.0 mmol) in ethanol (8 ml) was heated at 70° C. for 7 days. The reaction mixture was loaded on to an SCX-cartridge and eluted with methanol, followed by ammonia in methanol (2M). The filtrate was concentrated in vacuo and the residue was purified by automated preparative liquid chromatography (Gilson system, 250 mm×50 mm Gemini C18 10 μm column, 120 ml/min) using an acetonitrile: 0.1% aqueous:ammonia (5:95): acetonitrile:0.1% aqueous ammonia (95:5) gradient [90:10 to 80:20 (from 2 to 6 min) to 60:40 (from 15 to 16 min) to 5:95 (from 16 to 17 min)]. The appropriate fractions were combined and concentrated to give the compound of Example 44 (52 mg) as a single enantiomer. HPLC Method A—retention time 11.26 min.

Experimental MH⁺ 368.2; expected 368.2

¹H-NMR (CD₃OD): 1.16-1.18 (3H), 2.55-2.59 (2H), 4.66-4.68 (1H), 6.57-6.60 (1H), 7.00-7.02 (1H), 7.04-7.07 (1H), 7.22-7.24 (1H), 7.31-7.33 (1H), 7.74-7.76 (1H)

Bovine EC₅₀—1.3 nM; Porcine EC₅₀—1.8 nM

Similarly prepared were:

### Example 45

### (6R,7R)-6-{[(1R)-3-(5-Aminopyridin-3-yl)-1-methylpropyl]amino}-7-hydroxy-4,5,6,7-tetrahydroimidazo[4,5,1-jk][1]benzazepin-2(1H)-one

From the compound of Preparation 78, as a single enantiomer.

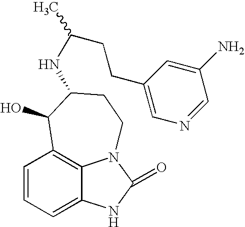

HPLC Method A—retention time 10.25 min.

Experimental MH⁺ 368.2; expected 368.2

¹H-NMR (CD₃OD): 1.16-1.18 (3H), 2.60-2.64 (2H), 4.66-4.68 (1H), 6.95-6.96 (1H), 6.99-7.01 (1H), 7.04-7.07 (1H), 7.20-7.22 (1H), 7.65-7.66 (1H), 7.77-7.79 (1H)

Bovine EC₅₀—1.4 nM; Porcine EC₅₀—1.1 nM

### Example 46

### (6R,7R)-6-{[(1R)-3-(6-Aminopyridin-3-yl)-1-methylpropyl]amino}-7-hydroxy-4,5,6,7-tetrahydroimidazo[4,5,1-jk][1]benzazepin-2(1H)-one

From the compound of Preparation 79, as a single enantiomer.

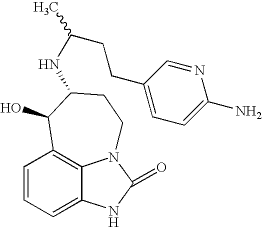

HPLC Method A—retention time 10.47 min.

Experimental MH⁺ 368.2; expected 368.2

¹H-NMR (CD₃OD): 1.13-1.16 (3H), 2.52-2.57 (2H), 4.64-4.66 (1H), 6.53-6.55 (1H), 6.99-7.01 (1H), 7.03-7.07 (1H), 7.20-7.22 (1H), 7.35-7.37 (1H), 7.60-7.62 (1H)

Bovine EC₅₀—3 nM; Porcine EC₅₀—2.8 nM

### Example 47

### (6R,7R)-7-Hydroxy-6-{[(1R)-3-isothiazol-4-yl-1-methylpropyl]amino}-4,5,6,7-tetrahydroimidazo[4,5,1-jk][1]benzazepin-2(1H)-one

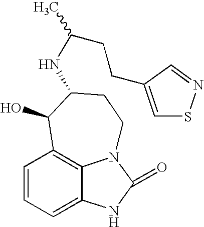

To a mixture of the compound of Preparation 9 (108 mg, 0.4 mmol) and triethylamine (18 μl, 0.1 mmol) in methanol (5 ml) was added the compound of Preparation 15 (79 mg, 0.5 mmol), followed by sodium cyanoborohydride (40 mg, 0.6 mmol) and the reaction mixture was heated at 50° C. for 18 h. After cooling, the mixture was quenched by addition of water (3 ml) and citric acid was added, followed by excess sodium hydrogen carbonate. The mixture was stirred at room temperature for 30 min and then concentrated in vacuo. The residue was passed through a silica plug, eluting with dichloromethane:2.5% methanolic ammonia [4:1] and the filtrate was concentrated in vacuo. The residue was purified by automated preparative liquid chromatography (Gilson system, 150 mm×21 mm Gemini C18 5 μm column, 25 ml/min) using an acetonitrile: 0.1% aqueous ammonia gradient [5:95 to 20:80 (from 0 to 6 min) to 98:2 (from 8 to 8.5 min)]. The appropriate fractions were combined and concentrated to give the compound of Example 47 (30 mg) as a single enantiomer. HPLC Method A—retention time 11.63 min.

Experimental MH⁺ 359.1; expected 359.2

¹H-NMR (CD₃OD): 1.14-1.16 (3H), 4.64-4.66 (1H), 6.99-7.01 (1H), 7.02-7.05 (1H), 7.10-7.12 (1H), 8.39-8.40 (1H), 8.56-8.57 (1H)

Bovine EC₅₀—0.6 nM; Porcine EC₅₀—0.9 nM

### Example 48

### (6R,7R)-7-Hydroxy-6-{[(1R)-1-methyl-3-(1,3,5-trimethyl-1H-pyrazol-4-yl)propyl]amino}-4,5,6,7-tetrahydroimidazo[4,5,1-jk][1]benzazepin-2(1H)-one

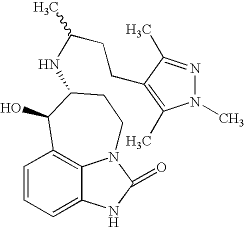

To a mixture of the compound of Preparation 9 (472 mg, 1.9 mmol) and triethylamine (77 μl, 0.6 mmol) in methanol (15 ml) was added the compound of Preparation 70 (400 mg, 2.2 mmol), followed by sodium cyanoborohydride (174 mg, 2.8 mmol) and the reaction mixture was heated at 50° C. for 18 h. After cooling, the mixture was quenched by addition of water (3 ml) and citric acid was added, followed by excess sodium hydrogen carbonate. The mixture was stirred at room temperature for 30 min and then concentrated in vacuo. To the residue was added methanol (250 ml) and silica and the mixture was concentrated in vacuo. The product/silica mix was dry loaded on to silica and eluted with dichloromethane:2.5% methanolic ammonia [4:1]. The appropriate fractions were concentrated in vacuo and the residue was purified by automated flash chromatography (Biotage™, 40+M silica cartridge) with gradient elution, dichloromethane:2.5% methanolic ammonia [96:4 to 91:9]. The appropriate fractions were combined and concentrated and the residue was dissolved in acetonitrile:water (9:1, 2 ml) and further purified by automated preparative liquid chromatography (Gilson system, 150 mm×21.4 mm Gemini C18 5 μm column, 20 ml/min) using an acetonitrile: 0.1% aqueous ammonia (5:95): acetonitrile: 0.1% aqueous ammonia (95:5) gradient [90:10 to 78:22 (from 2 to 15 min) to 88:22 (from 15 to 20 min) to 50:50 (from 20 to 25 min) to 5:95 (from 25 to 26 min]. The appropriate fractions were combined and concentrated to give the compound of Example 48 (100 mg) as a single enantiomer.

HPLC Method A—retention time 11.84 min.

Experimental MH⁺ 384.5; expected 384.2

¹H-NMR (CD₃OD): 1.12-1.15 (3H), 2.08-2.10 (2H), 2.14-2.16 (2H), 3.62-3.64 (3H), 4.61-4.63 (1H), 6.99-7.01 (1H), 7.02-7.05 (1H), 7.18-7.20 (1H)

Bovine EC₅₀—3.4 nM; Porcine EC₅₀—3.1 nM

### Example 49

### (6R,7R)-7-Hydroxy-6-{[(1R)-1-methyl-3-(1,3-thiazol-5-yl)propyl]amino}-4,5,6,7-tetrahydroimidazo[4,5,1-jk][1]benzazepin-2(1H)-one

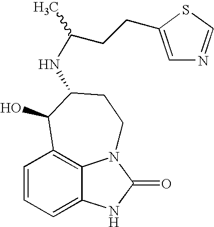

To a mixture of the compound of Preparation 9 (821 mg, 3.2 mmol) and the compound of Preparation 14 (498 mg, 3.2 mmol) in methanol (25 ml) was added triethylamine (134 μl, 1.0 mmol) and the mixture was heated at 50° C. After 10 min, sodium cyanoborohydride (303 mg, 4.8 mmol) was added and the reaction mixture was heated at 50° C. for 18 h. After cooling, the mixture was concentrated in vacuo and to the residue was added methanol (50 ml). The solution was concentrated in vacuo and the process was repeated with methanol (2×50 ml) a further two times. The residue was dissolved in dichloromethane (10 ml) and methanol (1 ml) and purified by automated flash chromatography (Biotage™, 40M silica cartridge) with gradient elution, dichloromethane:2.5% methanolic ammonia [92:8 to 88:12]. The appropriate fractions were combined and concentrated and the residue was dissolved in acetonitrile:water (9:1, 4 ml) and further purified by automated preparative liquid chromatography (Gilson system, 150 mm×21.4 mm Gemini C18 5 μm column, 20 ml/min) using an acetonitrile: 0.1% aqueous ammonia (5:95): acetonitrile:0.1% aqueous ammonia (95:5) gradient [90:10 to 75:25 (from 2 to 8 min) to 50:50 (from 24 to 26 min) to 5:95 (from 26 to 27 min]. The appropriate fractions were combined and concentrated to give the compound of Example 49 (266 mg) as a single enantiomer. HPLC Method A—retention time 11.03 min.

Experimental MH⁺ 359.1; expected 359.2

¹H-NMR (CD₃OD): 1.16-1.18 (3H), 2.97-3.01 (2H), 4.63-4.65 (1H), 6.98-7.00 (1H), 7.02-7.05 (1H), 7.10-7.12 (1H), 7.61-7.62 (1H), 8.80-8.81 (1H)

Bovine EC₅₀—4.7 nM; Porcine EC₅₀—6.4 nM

### Example 50

### (6R,7R)-7-Hydroxy-6-{[3-(1H-indol-3-yl)-1-methylpropyl]amino}-4,5,6,7-tetrahydroimidazo[4,5,1-jk][1]benzazepin-2(1H)-one

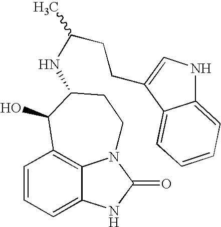

To a mixture of the compound of Preparation 9 (10.0 g, 39.0 mmol) and the compound of Preparation 43 (8.8 g, 46.9 mmol) in methanol (90 ml), under nitrogen, was added triethylamine (1.6 ml, 11.7 mmol) and the mixture was stirred at room temperature. After 20 min, sodium cyanoborohydride (3.7 g, 58.7 mmol) was added and the reaction mixture was heated at 60° C., under nitrogen, for 18 h. The mixture was distilled to remove the solvent and to the residue was added methanol (100 ml). The mixture was filtered, washing through with methanol, and the filtrate was concentrated in vacuo to give the compound of Example 50 (18.0 g) as a mixture of two non-racemic diastereoisomers.

¹H-NMR (CD₃OD): 1.17-1.23 (3H), 4.59-4.63 (1H), 6.90-6.95 (1H), 6.98-7.08 (3H), 7.10-7.13 (1H), 7.29-7.32 (1H), 7.46-7.50 (1H)

Bovine EC₅₀—N/A; Porcine EC₅₀—N/A

The following Examples were prepared by similar methods to those described above for Examples 47-50:

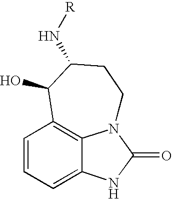

| | | | MH⁺ | EC₅₀ (nM) | From the |
|---|---|---|---|---|---|
| | | Structure | Found/ | Bovine/ | compound of |
| Example | R | Comment | Expected | Porcine | Preparation: |
| 51 | 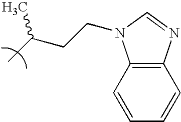 | Single Enantiomer | 392.2 392.2 | 0.6 0.9 | 84 |
| 52 | 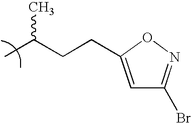 | Single Enantiomer | N/A | 1.2 1.3 | 35 |
| 53 | 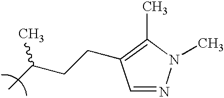 | Single Enantiomer | 370.2 370.2 | 2 2.8 | 25 |
| 54 | 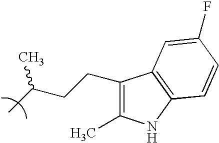 | Single Enantiomer | 423.3 423.2 | 2.7 3.1 | 85 |
| 55 | 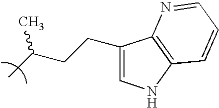 | Single Enantiomer | 392.2 392.2 | 4.4 9.9 | 86 |
| 56 | 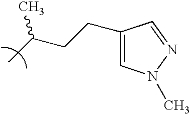 | Single Enantiomer | 356.2 356.2 | 5.2 2.7 | 26 |
| 57 | 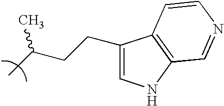 | Single Enantiomer | 392.2 392.2 | 5.7 13 | 47 |
| 58 | 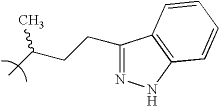 | Single Enantiomer | 392.4 392.2 | 8.3 33 | 51 |
| 59 | 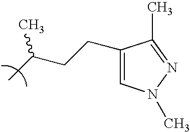 | Single Enantiomer | 370.1 370.2 | 20 17 | 27 |
| 60 | 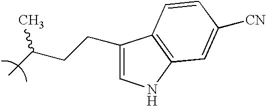 | Single Enantiomer | 416.2 416.2 | 23 13 | 87 |
| 61 | 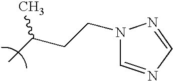 | Single Enantiomer | 343.2 343.2 | 28 18 | 88 |
| 62 | 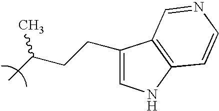 | Single Enantiomer | 392.1 392.2 | 35 28 | 48 |
| 63 | 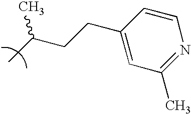 | Single Enantiomer | 367.2 367.2 | 81 25 | 28 |
| 64 | 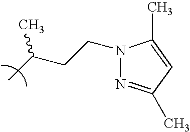 | Single Enantiomer | 370.2 370.2 | 169 188 | 89 |
| 65 | 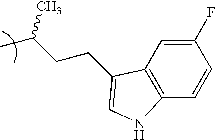 | Mixture of 2 non- racemic diastereoisomers | N/A | N/A | 50 |

### Example 51

### (6R,7R)-6-{[(1R)-3-(1H-Benzimidazol-1-yl)-1-methylpropyl]amino}-7-hydroxy-4,5,6,7-tetrahydroimidazo[4,5,1-jk][1]benzazepin-2(1H)-one

¹H-NMR (CD₃OD): 1.15-1.18 (3H), 4.38-4.44 (2H), 4.64-4.66 (1H), 6.99-7.01 (1H), 7.02-7.05 (1H), 7.10-7.12 (1H), 7.13-7.17 (2H), 7.52-7.54 (1H), 7.64-7.66 (1H), 8.15-8.16(1H)

HPLC Method A—retention time 11.69 min.

### Example 52

### (6R,7R)-6-{[(1R)-3-(3-Bromoisoxazol-5-yl)-1-methylpropyl]amino}-7-hydroxy-4,5,6,7-tetrahydroimidazo[4,5,1-jk][1]benzazepin-2(H)-one

¹H-NMR (d₆-DMSO): 0.99-1.02 (3H), 4.48-4.51 (1H), 6.83-6.85 (1H), 6.88-6.92 (2H), 7.01-7.03 (1H)

HPLC Method A—retention time 11.71 min.

### Example 53

### (6R,7R)-6-{[(1R)-3-(1,5-Dimethyl-1H-pyrazol-4-yl)-1-methylpropyl]amino}-7-hydroxy-4,5,6,7-tetrahydroimidazo[4,5,1-jk][1]benzazepin-2(H)-one

¹H-NMR (CD₃OD): 1.10-1.13 (3H), 2.17-2.18 (3H), 3.70-3.72 (3H), 4.62-4.64 (1H), 6.99-7.01 (1H), 7.03-7.07 (1H), 7.18-7.21 (2H)

HPLC Method A—retention time 11.19 min.

### Example 54

### (6R,7R)-6-{[(1R)-3-(5-Fluoro-2-methyl-1H-indol-3-yl)-1-methylpropyl]amino}-7-hydroxy-4,5,6,7-tetrahydroimidazo[4,5,1-jk][1]benzazepin-2(1H)-one

No n.m.r data available

HPLC Method A—retention time 14.91 min.

### Example 55

### (6R,7R)-7-Hydroxy-6-{[(1R)-1-methyl-3-(1H-pyrrolo[3,2-b]pyridin-3-yl)propyl]amino}-4,5,6,7-tetrahydroimidazo[4,51-jk][1]benzazepin-2(1H)-one

¹H-NMR (CD₃OD): 1.11-1.13 (3H), 1.90-1.96 (2H), 4.60-4.62 (1H), 6.57-6.59 (1H), 6.99-7.01 (1H), 7.02-7.05 (1H), 7.10-7.13 (1H), 7.17-7.19 (1H), 7.56-7.58 (1H), 7.83-7.85 (1H)

HPLC Method A—retention time 11.33 min.

### Example 56

### (6R,7R)-7-Hydroxy-6-{[(1R)-1-methyl-3-(1-methyl-1H-pyrazol-4-yl)propyl]amino}-4,5,6,7-tetrahydroimidazo[4,5,1-jk][1]benzazepin-2(H)-one

¹H-NMR (CD₃OD): 1.11-1.13 (3H), 3.79-3.80 (3H), 4.62-4.64 (1H), 6.99-7.01 (1H), 7.02-7.05 (1H), 7.19-7.21 (1H), 7.26-7.28 (1H), 7.36-7.38 (1H)

HPLC Method A—retention time 10.73 min.

### Example 57

### (6R,7R)-7-Hydroxy-6-{[(1R)-1-methyl-3-(1H-pyrrolo[2,3-c]pyridin-3-yl)propyl]amino}-4,5,6,7-tetrahydroimidazo[4,51-jk][1]benzazepin-2(1H)-one

¹H-NMR (CD₃OD): 1.14-1.17 (3H), 4.39-4.44 (2H), 4.60-4.62 (1H), 6.50-6.52 (1H), 6.98-7.00 (1H), 7.02-7.05 (1H), 7.18-7.20 (1H), 7.50-7.52 (1H), 7.56-7.58 (1H), 8.03-8.05 (1H)

HPLC Method A—retention time 11.72 min.

### Example 58

### (6R,7R)-7-Hydroxy-6-{[(1R)-3-(1H-indazol-3-yl)-1-methylpropyl]amino}-4,5,6,7-tetrahydroimidazo[4,5,1-jk][1]benzazepin-2(1H)-one

¹H-NMR (CD₃OD): 1.11-1.14 (3H), 2.05-2.10 (2H), 4.62-4.64 (1H), 6.99-7.01 (1H), 7.02-7.06 (2H), 7.17-7.19 (1H), 7.23-7.27 (1H), 7.54-7.56 (1H), 7.63-7.65 (1H)

HPLC Method A—retention time 12.47 min.

### Example 59

### (6R,7R)-6-{[(1R)-3-(1,3-Dimethyl-1H-pyrazol-4-yl)-1-methylpropyl]amino}-7-hydroxy-4,5,6,7-tetrahydroimidazo[4,5,1-jk][1]benzazepin-2(H)-one

¹H-NMR (CD₃OD): 1.11-1.13 (3H), 2.09-2.10 (3H), 3.72-3.74 (3H), 4.62-4.64 (1H), 6.99-7.01 (1H), 7.03-7.06 (1H), 7.19-7.21 (1H), 7.24-7.25 (1H)

HPLC Method A—retention time 10.90 min.

### Example 60

### 3-[(3R)-3-{[(6R,7R)-7-Hydroxy-2-oxo-1,2,4,5,6,7-hexahydroimidazo[4,5,1-jk][1]benzazepin-6-yl]amino}butyl]-1H-indole-6-carbonitrile

¹H-NMR (CD₃OD): 1.17-1.19 (3H), 1.75-1.80 (2H), 4.62-4.64 (1H), 6.98-7.00 (1H), 7.02-7.05 (1H), 7.16-7.18 (1H), 7.21-7.23 (1H), 7.28-7.29 (1H), 7.64-7.66 (1H), 7.70-7.71 (1H)

HPLC Method A—retention time 13.50 min.

### Example 61

### (6R,7R)-7-Hydroxy-6-{[(1R)-1-methyl-3-(1H-1,2,4-triazol-1-yl)propyl]amino}-4,5,6,7-tetrahydroimidazo[4,5,1-jk][1]benzazepin-2(1H)-one

¹H-NMR (CD₃OD): 1.14-1.16 (3H), 4.63-4.65 (1H), 6.98-7.00 (1H), 7.02-7.05 (1H), 7.20-7.22 (1H), 7.93-7.94 (1H)

HPLC Method A—retention time 9.39 min.

### Example 62

### (6R,7R)-7-Hydroxy-6-{[(1R)-1-methyl-3-(1H-pyrrolo[3,2-c]pyridin-3-yl)propyl]amino}-4,5,6,7-tetrahydroimidazo[4,51-jk][1]benzazepin-2(1H)-one

¹H-NMR (CD₃OD): 1.13-1.15 (3H), 4.28-4.35 (2H), 4.61-4.63 (1H), 6.61-6.63 (1H), 6.99-7.01 (1H), 7.02-7.05 (1H), 7.18-7.20 (1H), 7.36-7.38 (1H), 7.40-7.42 (1H), 8.06-8.08 (1H)

HPLC Method A—retention time 11.19 min.

### Example 63

### (6R,7R)-7-Hydroxy-6-{[(1R)-1-methyl-3-(2-methylpyridin-4-yl) propyl]amino}-4,5,6,7-tetrahydroimidazo[4,5,1-jk][1]benzazepin-2(1H)-one

¹H-NMR (CD₃OD): 1.16-1.18 (3H), 2.46-2.48 (3H), 4.63-4.65 (1H), 6.99-7.01 (1H), 7.03-7.09 (2H), 7.17-7.18 (1H), 7.19-7.21 (1H), 8.22-8.24 (1H)

HPLC Method A—retention time 11.42 min.

### Example 64

### (6R,7R)-6-{[(1R)-3-(3,5-Dimethyl-1H-pyrazol-1-yl)-1-methylpropyl]amino}-7-hydroxy-4,5,6,7-tetrahydroimidazo[4,51-jk][1]benzazepin-2(1H)-one

¹H-NMR (CD₃OD): 1.11-1.13 (3H), 2.10-2.12 (3H), 2.18-2.20 (3H), 4.62-4.64 (1H), 5.78-5.79 (1H), 6.99-7.01 (1H), 7.02-7.06 (1H), 7.18-7.20 (1H)

HPLC Method A—retention time 11.53 min.

### Example 65

### (6R,7R)-6-{[3-(5-Fluoro-1H-indol-3-yl)-1-methylpropyl]amino}-7-hydroxy-4,5,6,7-tetrahydroimidazo[4,5,1-jk][1]benzazepin-2(1H)-one

¹H-NMR (d₆-DMSO): 1.00-1.08 (3H), 4.80-4.83 (1H), 7.05-7.10 (2H), 7.27-7.30 (2H), 7.37-7.39 (1H), 7.40-7.44 (1H), 7.70-7.72 (1H)

Mixture of non-racemic diastereoisomers.

### Preparation 1

Hydrochloride salt of 6-Amino-7-hydroxy-4,5,6,7-tetrahydroimidazo[4,5,1-jk][1]benzazepin-2(1H)-one

To a solution of the compound of Preparation 2 (53.5 g, 211.0 mmol) in methanol (2600 ml), at 0° C., was added sodium borohydride (8.8 g, 232.1 mmol), over 30 min. The reaction mixture was stirred at room temperature for 18 h, before addition of hydrochloric acid (2N, 120 ml). The mixture was concentrated in vacuo and the residue was re-crystallised from isopropyl alcohol:water (3:1, 700 ml). The solid was washed with diethyl ether and dried in a vacuum oven overnight to give the title compound (33.8 g).

¹H-NMR (d₆-DMSO): 2.00-2.10 (1H), 2.30-2.40 (1H), 3.60-3.70 (1H), 4.10-4.20 (1H), 4.85-4.95 (1H), 6.45-6.50 (1H), 6.90-6.95 (1H), 6.95-7.00 (1H), 7.15-7.20 (1H)

### Preparation 2

### Hydrochloride salt of 6-Amino-5,6-dihydroimidazo[4,5,1-jk][1]benzazepine-2,7(1H,4H)-dione

A mixture of the compound of Preparation 3 (35.3 g, 153.0 mmol), palladium (10% on carbon, 11.0 g) and concentrated hydrochloric acid (25.5 ml) in methanol (300 ml) was stirred at room temperature under hydrogen (22 psi) for 3 h. The reaction mixture was filtered through Arbocel®, washing through with methanol and water, and ensuring the catalyst did not dry out. The filtrate was concentrated in vacuo and the residue was triturated with acetone to give the title compound (30.0 g).

¹H-NMR (d₆-DMSO): 2.20-2.30 (1H), 2.40-2.50 (1H), 3.70-3.80 (1H), 4.30-4.40 (1H), 4.60-4.70 (1H), 7.10-7.15 (1H), 7.25-7.30 (1H), 7.60-7.65 (1H)

### Preparation 3

### 4,5-Dihydroimidazo[4,5,1-jk][1]benzazepine-2,6,7(1H)-trione 6-oxime

To a solution of the compound of Preparation 4 (10.3 g, 51.0 mmol) in acetic acid (150 ml) was added tert-butyl nitrite (16 ml, 135.0 mmol), followed by hydrochloric acid (4N in dioxane, 33.4 ml). The reaction mixture was stirred at room temperature for 3 h and then filtered. The solid material was dried in a vacuum oven to give the title compound (10.0 g).

Experimental MH⁺ 232.1; expected 232.1

### Preparation 4

### 5,6-Dihydroimidazo[4,5,1-jk][1]benzazepine-2,7(1H,4H)-dione

To a solution of the compound of Preparation 5 (45.0 g, 0.2 mol) in dichloromethane (150 ml) was added thionyl chloride (30 ml, 0.4 mol) and the reaction mixture was stirred at room temperature for 2 h. The mixture was concentrated in vacuo and to the residue was added dichloromethane (1000 ml) and aluminium chloride (84.0 g, 0.6 mol), added portionwise. After stirring at room temperature overnight, the reaction mixture was heated at reflux for 2 h and then concentrated in vacuo. To the residue was added ice water (2000 ml) and concentrated hydrochloric acid (50 ml), followed by additional ice water (2000 ml). The resulting precipitate was collected by filtration, washed with water (4×250 ml) and dissolved in aqueous sodium hydroxide solution (1N, 600 ml). The solution was washed with dichloromethane (2×150 ml) and cyclohexane (150 ml) and adjusted to pH 10 by addition of dry ice. The solid material was collected by filtration, washed with water (3×50 ml) and dried overnight at 40° C. to give the title compound (30.0 g).

¹H-NMR (d₆-DMSO): 2.03-2.11 (2H), 2.90-3.00 (2H), 3.85-3.95 (2H), 7.02-7.10 (1H), 7.17-7.24 (1H), 7.50-7.58 (1H)

### Preparation 5

### 4-(2-Oxo-2,3-dihydro-1H-benzimidazol-1-yl)butanoic acid

To a solution of the compound of Preparation 6 (152.0 g, 0.6 mol) in tetrahydrofuran (600 ml) was added concentrated hydrochloric acid (75 ml). The reaction mixture was stirred for 2 h and then poured into water (700 ml). The mixture was filtered, washing through with water (750 ml), and the solid material was dried overnight at 40° C. to give the title compound (156.0 g).

¹H-NMR (d₆-DMSO): 1.80-1.89 (2H), 2.20-2.25 (2H), 3.74-3.82 (2H), 6.96-7.01 (3H), 7.05-7.10 (1H)

### Preparation 6

### 4-(3-Isopropenyl-2-oxo-2,3-dihydro-1H-benzimidazol-1-yl)butanoic acid

To a solution of the compound of Preparation 7 (223.8 g, 0.7 mol) in tetrahydrofuran (500 ml) was added aqueous sodium hydroxide solution (15% w/w, 500 ml). The reaction mixture was heated at reflux for 4 h, cooled to room temperature and stirred overnight. The tetrahydrofuran was removed by vacuum distillation (38° C.) and the aqueous layer was extracted with dichloromethane (2×400 ml) and cyclohexane (2×300 ml). To the aqueous layer was added glacial acetic acid (250 ml) and the solution was cooled to 2° C. After stirring for 30 min, the product was collected by filtration, washing through with water (3×250 ml), at 2° C. The solid was dried overnight at 40° C. to give the title compound (307.5 g).

Experimental MH⁺ 261.2; expected 261.1

### Preparation 7

### Ethyl 4-(3-isopropenyl-2-oxo-2,3-dihydro-1H-benzimidazol-1-yl)butanoate

A mixture of the compound of Preparation 8 (114.0 g, 0.7 mol), potassium carbonate (136 mg, 1.0 mol) and the compound of Preparation 130 (167.4 g, 0.9 mol) in acetone (500 ml) was heated at reflux for 18 h. The reaction mixture was then cooled to room temperature and filtered, washing through with acetone (250 ml). The filtrate was concentrated in vacuo and the residue was dried overnight at 40° C. to give the title compound (223.8 g).

¹H-NMR (d₆-DMSO): 1.10-1.20 (3H), 2.10-2.15 (3H), 3.95-4.07 (2H), 5.10-5.12 (1H), 5.35-5.39 (1H), 7.00-7.10 (3H), 7.20-7.26 (1H)

### Preparation 8

### 1-Isopropenyl-1,3-dihydro-2H-benzimidazol-2-one

To a solution of the compound of Preparation 102 (98.0 g, 0.9 mol) in xylene (420 ml), at 120° C., was added 1,8-diazobicylo[5.4.0]undec-7-ene (1.5 ml), followed by the compound of Preparation 125 (130.0 g, 1.0 mol), added over 30 min. The reaction mixture was heated at 150° C., using a Dean-Stark apparatus, for 60 h and then cooled to room temperature. The solid product was isolated by filtration, washing with cold xylene (250 ml), and dried in a vacuum oven to give the title compound (208.4 g).

¹H-NMR (d₆-DMSO): 2.08-2.11 (3H), 5.05-5.11 (1H), 5.34-5.37 (1H), 6.98-7.01 (3H), 7.01-7.06 (1H), 10.90-11.00 (1H)

### Preparation 9

### Hydrochloride salt of (6R,7R)-6-Amino-7-hydroxy-4,5,6,7-tetrahydroimidazo[4,5,1-jk][1]benzazepin-2(1H)-one

To the compound of Preparation 10 (160 mg, 0.5 mmol) was added hydrogen chloride (4N in dioxane, 1.3 ml, 5.0 mmol) and the mixture was stirred at room temperature for 1 h. The mixture was concentrated in vacuo and to the residue was added dioxane (10 ml). The solution was re-concentrated in vacuo to give the title compound (135 mg) as a single enantiomer.

¹H-NMR (CD₃OD): 2.07-2.13 (1H), 2.41-2.44 (1H), 3.50-3.54 (1H), 3.78-3.82 (1H), 4.20-4.26 (1H), 7.01-7.04 (1H), 7.10-7.14 (1H), 7.35-7.37 (1H)

Preferred Route

A mixture of the compound of Preparation 3 (11.0 g, 48.0 mmol), rhodium chloro(norbornadiene) dimer (55 mg, 0.1 mmol) and 1-[(S)-ferrocenyl-2-(R)-ethyl-1-dimethylamino)phenyl]-(S)-phosphino-1′-dicyclohexylphosphino-ferrocene (Solvias) (187 mg, 0.3 mmol) in methanol (165 ml) and water (11 ml) was purged with nitrogen (x 3) and heated at 80° C. under a hydrogen atmosphere (20 bar) for 16 h. The mixture was filtered, washed with methanol and concentrated in vacuo. To the residue was added hydrogen chloride (4M in dioxane, 14 ml). The solution was concentrated in vacuo and the residue was purified by azeotropic distillation with 2-propanol (2×50 ml). The residue was re-crystallised from 2-propanol:water (6:1, 150 ml) and again from 2-propanol:water (6:1, 80 ml) to give the title compound (6.5 g).

¹H-NMR (d₆-DMSO): 1.96-2.05 (1H), 2.30-2.38 (1H), 3.60-3.68 (1H), 4.08-4.15 (1H), 4.82-4.88 (1H), 6.45-6.50 (1H), 6.90-6.93 (1H), 6.97-7.01 (1H), 7.15-7.18 (1H)

### Preparation 10

### tert-Butyl [(6R,7R)-7-hydroxy-2-oxo-1,2,4,5,6,7-hexahydroimidazo[4,5,1-jk][1]benzazepin-6-yl]carbamate

The compound of Preparation 11 (500 mg, 1.6 mmol) was dissolved in 2-propanol containing 0.1% diethylamine (100 ml), with heating and sonicating. The solution was purified by supercritical fluid chromatography (Berger Multigram III, 250×30 mm Chiralcel OJ-H, 5 □m column, 35° C., 180 ml/min) using carbon dioxide: 2-propanol containing 0.1% diethylamine [85:15] as the mobile phase. The appropriate fractions were combined and concentrated to give the title compound as the desired enantiomer, which was used directly.

### Preparation 11

### tert-Butyl [7-hydroxy-2-oxo-1,2,4,5,6,7-hexahydroimidazo[4,5,1-jk][1]benzazepin-6-yl]carbamate

To a solution of the compound of Preparation 1 (1.0 g, 3.9 mmol) in methanol (20 ml) was added triethylamine (1.1 ml, 7.8 mmol), followed by the compound of Preparation 106 (1.7 g, 7.8 mmol). The reaction mixture was stirred for 1 h, concentrated in vacuo and to the residue was added dichloromethane (50 ml). This solution was washed with water (50 ml) and the precipitate was collected by filtration. The resulting solid was dried in a vacuum oven to give the title compound (500 mg), which was used directly.

### Preparation 12

### 4-(6-Methoxypyridin-2-yl)butan-2-one

A mixture of the compound of Preparation 29 (1.4 g, 7.9 mmol) and palladium (10 wt. % on carbon, 100 mg) in methanol (10 ml) was stirred under a hydrogen atmosphere (60 psi) for 1 h. The reaction mixture was filtered through Arbocel®, washing through with methanol, and the filtrate was concentrated in vacuo to give the title compound (940 mg).

Experimental MH⁺ 180.2; expected 180.1

### Preparation 13

### 4-(5-Fluoro-1H-indol-7-yl)butan-2-one

A mixture of the compound of Preparation 30 (8.9 g, 43.9 mmol) and the compound of Preparation 152 (1.0 g, 1.1 mmol) in ethyl acetate (120 ml) was stirred at room temperature, under hydrogen (1 atm), for 60 h. The mixture was filtered through Celite® and the filtrate was concentrated in vacuo. A portion of the residue was dissolved in dichloromethane (5 ml) and purified by flash chromatography (silica), with gradient elution, cyclohexane:ethyl acetate [98:2 to 50:50]. The appropriate fractions were combined and concentrated to give the title compound (836 mg).

¹H-NMR (CD₃OD): 2.13-2.15 (3H), 2.86-2.92 (2H), 3.05-3.11 (2H), 6.38-6.41 (1H), 6.67-6.72 (1H), 7.00-7.05 (1H), 7.24-7.26 (1H)

### Preparation 14

### 4-(1,3-Thiazol-5-yl)butan-2-one

A mixture of the compound of Preparation 55 (4.0 g, 21.0 mmol) and palladium (5 wt. % on alumina, 0.8 g) in ethanol (60 ml) was stirred at room temperature, under hydrogen (60 psi), for 18 h. The mixture was filtered and the filtrate was concentrated in vacuo. To the residue was added acetonitrile (30 ml) and the solution was washed with heptane (2×25 ml) and concentrated in vacuo to give the title compound (3.0 g).

¹H-NMR (CDCl₃): 2.16-2.19 (3H), 2.79-2.83 (2H), 3.09-3.13 (2H), 7.58-7.60 (1H), 8.60-8.62 (1H)

Similarly prepared were:

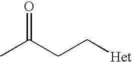

| | | From the |
|---|---|---|
| Preparation | Het | Compound of: |
| 15 | 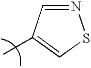 | Preparation 31 |
| 16 | 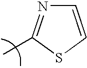 | Preparation 56 |
| 17 | 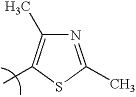 | Preparation 57 |
| 18 | 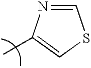 | Preparation 58 |
| 19 | 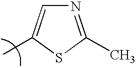 | Preparation 59 |
| 20 | 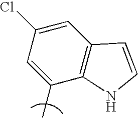 | Preparation 54 |
| 21 | 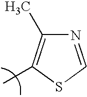 | Preparation 61 |
| 22 | 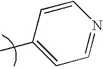 | Preparation 62 |
| 23 | 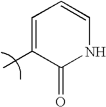 | Preparation 34 |
| 24 | 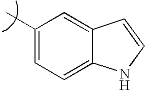 | Preparation 60 |
| 25 | 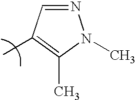 | Preparation 63 |
| 26 | 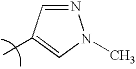 | Preparation 64 |
| 27 | 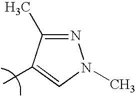 | Preparation 65 |
| 28 | 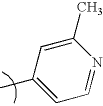 | Preparation 66 |

### Preparation 15

### 4-Isothiazol-4-ylbutan-2-one

¹H-NMR (CDCl₃): 2.17-2.19 (3H), 2.78-2.81 (2H), 2.97-3.00 (2H), 8.29-8.30 (1H), 8.36-8.37 (1H)

### Preparation 16

### 4-(1,3-Thiazol-2-yl)butan-2-one

¹H-NMR (CDCl₃): 2.17-2.20 (3H), 2.96-3.02 (2H), 3.25-3.30 (2H), 7.15-7.19 (1H), 7.61-7.65 (1H)

### Preparation 17

### 4-(2,4-Dimethyl-1,3-thiazol-5-yl)butan-2-one

¹H-NMR (CDCl₃): 2.12-2.15 (3H), 2.27-2.30 (3H), 2.56-2.60 (3H), 2.67-2.74 (2H), 2.90-2.96 (2H)

### Preparation 18

### 4-(1,3-Thiazol-4-yl)butan-2-one

¹H-NMR (CDCl₃): 2.14-2.17 (3H), 2.88-2.93 (2H), 3.06-3.12 (2H), 6.98-7.01 (1H), 8.71-8.74 (1H)

### Preparation 19

### 4-(2-Methyl-1,3-thiazol-5-yl)butan-2-one

¹H-NMR (CD₃OD): 2.13-2.15 (3H), 2.60-2.62 (3H), 2.81-2.85 (2H), 3.00-3.05 (2H), 7.28-7.31 (1H)

### Preparation 20

### 4-(5-Chloro-1H-indol-7-yl)butan-2-one

¹H-NMR (CDCl₃): 2.14-2.16 (3H), 2.88-2.94 (2H), 3.06-3.11 (2H), 6.44-6.47 (1H), 6.90-6.93 (1H), 7.22-7.26 (1H), 7.45-7.47 (1H)

### Preparation 21

### 4-(4-Methyl-1,3-thiazol-5-yl)butan-2-one

¹H-NMR (CDCl₃): 2.17-2.18 (3H), 2.40-2.41 (3H), 2.70-2.88 (2H), 3.00-3.06 (2H), 8.55-8.56 (1H)

### Preparation 22

### 4-Pyridin-4-ylbutan-2-one

¹H-NMR (CDCl₃): 2.13-2.17 (3H), 2.75-2.81 (2H), 2.85-2.91 (2H), 7.08-7.13 (2H), 8.46-8.51 (2H)

### Preparation 23

### 3-(3-Oxobutyl)pyridin-2(1H)-one

¹H-NMR (CD₃OD): 2.11-2.14 (3H), 2.57-2.61 (1H), 2.70-2.76 (2H), 2.78-2.81 (1H), 6.29-6.35 (1H), 7.23-7.26 (1H), 7.41-7.44 (1H)

### Preparation 24

### 4-(1H-Indol-5-yl)butan-2-one

¹H-NMR (CD₃OD): 2.07-2.09 (3H), 2.77-2.80 (2H), 2.86-2.89 (2H), 6.36-6.38 (1H), 6.90-6.92 (1H), 7.17-7.18 (1H), 7.24-7.26 (1H), 7.33-7.35 (1H)

### Preparation 25

### 4-(1,5-Dimethyl-1H-pyrazol-4-yl)butan-2-one

¹H-NMR (CDCl₃): 2.05-2.07 (3H), 2.10-2.12 (3H), 2.57-2.62 (4H), 3.65-3.67 (3H), 7.16-7.18 (1H)

### Preparation 26

### 4-(1-Methyl-1H-pyrazol-4-yl)butan-2-one

Experimental MH⁺ 153.2; expected 153.1

### Preparation 27

### 4-(1,3-Dimethyl-1H-pyrazol-4-yl)butan-2-one

Experimental MH⁺ 167.0; expected 167.1

### Preparation 28

### 4-(2-Methylpyridin-4-yl)butan-2-one

Experimental MH⁺ 164.3; expected 164.1

### Preparation 29

### 4-(6-Methoxypyridin-2-yl)but-3-en-2-one

To a solution of the compound of Preparation 68 (1.0 g, 7.3 mmol) in acetone (3.2 ml, 43.8 mmol), at 0° C., was added aqueous sodium hydroxide solution (5M, 2.2 ml). The reaction mixture was stirred at 0° C. for 1 h and then at room temperature for 18 h. The mixture was acidified with hydrochloric acid (4M, 4 ml) and then neutralised with sodium hydrogen carbonate. The mixture was extracted with ethyl acetate and the combined organic extracts were concentrated in vacuo to give the title compound (1.4 g).

¹H-NMR (CDCl₃): 2.40-2.42 (3H), 3.89-3.94 (3H), 6.73-6.78 (1H), 6.98-7.02 (1H), 7.37-7.44 (1H), 7.48-7.61 (3H)

### Preparation 30

### 4-(5-Fluoro-1H-indol-7-yl)but-3-en-2-one

A mixture of the compound of Preparation 36 (9.8 g, 60.0 mmol) and the compound of Preparation 93 (38.4 g, 121.0 mmol) in tetrahydrofuran (100 ml) was heated at reflux for 18 h. The mixture was concentrated in vacuo and the residue was partitioned between diethyl ether and water. The two layers were separated and the organic phase was washed with water and brine, dried (MgSO₄) and concentrated in vacuo. The residue was triturated with diethyl ether and the solid material was removed by filtration. The filtrate was concentrated in vacuo and the residue was dissolved in dichloromethane (80 ml) and purified by automated flash chromatography (Biotage™ 65i cartridge), with gradient elution, cyclohexane:ethyl acetate [98:2 to 50:50]. The appropriate fractions were combined and concentrated to give the title compound (836 mg).

¹H-NMR (CD₃OD): 2.43-2.45 (3H), 6.49-6.53 (1H), 6.85-6.91 (1H), 7.24-7.38 (3H), 8.04-8.11 (1H)

### Preparation 31

### 4-Isothiazol-4-ylbut-3-en-2-one

To a solution of the compound of Preparation 80 (900 mg, 8.0 mmol) in tetrahydrofuran (32 ml) was added the compound of Preparation 93 (5.1 g, 15.9 mmol) and the reaction mixture was heated at reflux for 3 h. The mixture was concentrated in vacuo and the residue was triturated with diethyl ether. The solution was filtered, washing through with diethyl ether, and the filtrate was concentrated in vacuo to give the title compound (1.2 g).

¹H-NMR (CDCl₃): 2.38-2.39 (3H), 6.66-6.67 (1H), 6.70-6.71 (1H), 8.70-8.71 (1H), 8.78-8.79 (1H)

Similarly prepared were:

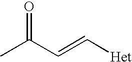

| | | From the |
|---|---|---|
| Preparation | Het | Compound of: |
| 32 | 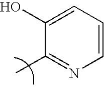 | Preparation 119 |
| 33 | 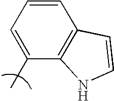 | Preparation 98 |
| 34 | 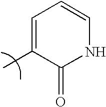 | Preparation 129 |
| 35 | 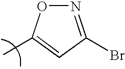 | Preparation 81 |

### Preparation 32

### 4-(3-Hydroxypyridin-2-yl)but-3-en-2-one

Experimental MH⁺ 163.9; expected 164.1

### Preparation 33

### 4-(1H-Indol-7-yl)but-3-en-2-one

¹H-NMR (CD₃OD): 2.43-2.45 (3H), 6.51-6.54 (1H), 6.86-6.92 (1H), 7.04-7.10 (1H), 7.30-7.33 (1H), 7.45-7.50 (1H), 7.63-7.67 (1H), 8.09-8.16 (1H)

### Preparation 34

### 3-(3-Oxo-butyl)-1H-pyridin-2-one

¹H-NMR (CD₃OD): 2.35-2.40 (3H), 6.41-6.46 (1H), 7.18-7.24 (1H), 7.50-7.53 (1H), 7.59-7.65 (1H), 7.89-7.93 (1H)

### Preparation 35

### 4-(3-Bromoisoxazol-5-yl)but-3-en-2-one

¹H-NMR (CDCl₃): 2.38-2.39 (3H), 6.82-6.84 (2H), 7.23-7.25 (1H), 7.50-7.52 (1H)

### Preparation 36

### 5-Fluoro-1H-indole-7-carbaldehyde

To a solution of the compound of Preparation 38 (36.7 g, 125.0 mmol) in tetrahydrofuran (300 ml) was added hydrochloric acid (1M, 30 ml) and the reaction mixture was stirred for 20 min. The mixture was neutralised with aqueous sodium hydrogen carbonate solution and extracted with diethyl ether. The combined extracts were washed with brine, dried (MgSO₄) and concentrated in vacuo and the residue was azeotroped with toluene to give the crude product. The crude product was pre-absorbed on to silica and purified by column chromatography (silica, 400 g, pre-wet with cyclohexane) with gradient elution, cyclohexane:ethyl acetate [100:0 to 85:15]. The appropriate fractions were combined and concentrated to give the title compound (9.8 g).

¹H-NMR (CDCl₃): 6.58-6.62 (1H), 7.38-7.42 (2H), 7.59-7.64 (1H), 10.06-10.08 (1H)

Similarly prepared was:

### Preparation 37

### 5-Chloro-1H-indole-7-carbaldehyde

From the compound of Preparation 39.

¹H-NMR (CDCl₃): 6.56-6.59 (1H), 7.35-7.39 (1H), 7.58-7.61 (1H), 7.86-7.90 (1H), 10.04-10.06 (1H)

### Preparation 38

### 7-(Dibutoxymethyl)-5-fluoro-1H-indole

To a solution of the compound of Preparation 41 (33.0 g, 110 mmol) in tetrahydrofuran (400 ml), at −70° C. and under nitrogen, was added dropwise the compound of Preparation 131 (1M in tetrahydrofuran, 330 ml), using a canula. After stirring at −70° C. for 1 h, the mixture was quenched by addition of aqueous ammonium chloride solution. The mixture was extracted with diethyl ether and the combined extracts were washed with brine, dried (MgSO₄) and concentrated in vacuo to give the title compound (36.7 g) which was used directly in the next stage.

Similarly prepared was:

### Preparation 39

### 5-Chloro-7-(dibutoxymethyl)-1H-indole

From the compound of Preparation 40.

The title compound (10.9 g) was used directly in the next stage

### Preparation 40

### 4-Chloro-2-(dibutoxymethyl)-1-nitrobenzene

A mixture of the compound of Preparation 104 (10.6 g, 57.0 mmol), p-toluenesulphonic acid (500 mg, 3.0 mmol) and the compound of Preparation 91 (16 ml, 171.0 mmol) in toluene (110 ml) was heated at reflux for 4 h and then stirred at room temperature for 18 h. The mixture was concentrated in vacuo and the residue was partitioned between ethyl acetate and water. The two layers were separated and the organic phase was washed with brine, dried (MgSO₄) and concentrated in vacuo. The residue was purified by column chromatography (silica), eluting with dichloromethane and the appropriate fractions were combined and concentrated to give the title compound (10.1 g).

¹H-NMR (CDCl₃): 0.88-0.97 (6H), 1.34-43(4H), 1.50-1.62 (4H), 3.50-3.65 (4H), 7.40-7.43 (1H), 7.79-7.81 (2H)

### Preparation 41

### 2-(Dibutoxymethyl)-4-fluoro-1-nitrobenzene

A mixture of the compound of Preparation 111 (6.6 g, 39.3 mmol), the compound of Preparation 91 (8.7 g, 118.0 mmol) and p-toluenesulphonic acid (400 mg, 2.0 mmol) in toluene (70 ml) was heated at reflux for 18 h and then stirred at room temperature for 18 h. The mixture was concentrated in vacuo and the residue was azeotroped with ethyl acetate and then partitioned between ethyl acetate and water. The two layers were separated and the organic phase was washed with brine, dried (MgSO₄) and concentrated in vacuo to give the title compound (11.4 g).

¹H-NMR (CDCl₃): 0.94-0.96 (6H), 1.39-1.42 (4H), 1.58-1.62 (4H), 3.55-3.65 (4H), 7.10-7.12 (1H), 7.54-7.56 (1H), 7.88-7.90 (1H)

### Preparation 42

### 4-(5-Chloro-1H-indol-3-yl)butan-2-one

To a mixture of the compound of Preparation 108 (2.5 g, 16.6 mmol) and the compound of Preparation 94 (1.4 ml, 16.6 mmol) in dichloromethane (10 ml) was added indium (III) chloride (333 mg, 1.5 mmol). After stirring for 90 min, the mixture was purified by automated flash chromatography (Biotage™ 65i cartridge conditioned with 15% ethyl acetate:cyclohexane) with gradient elution, ethyl acetate:cyclohexane [15:85 to 25:75]. The appropriate fractions were combined and concentrated to give the title compound (3.1 g).

¹H-NMR (CD₃OD): 2.08-2.10 (3H), 2.79-2.84 (2H), 2.89-2.94 (2H), 6.98-7.04 (2H), 7.22-7.26 (1H), 7.45-7.47 (1H)

### Preparation 43

### 4-(1H-Indol-3-yl)butan-2-one

To a solution of the compound of Preparation 121 (82.0 g, 700 mmol) in acetonitrile (1.5 l), under nitrogen, was added bismuth (III) triflate (13.8 g, 21.0 mmol) and the compound of Preparation 94 (58.3 ml, 49.1 g, 700 mmol). The reaction mixture was stirred at room temperature for 3 h and then partially concentrated in vacuo. To the residue was added water (800 ml) and the slurry was extracted with ethyl acetate (400 ml). The combined organic extracts were washed with brine, dried (MgSO₄) and concentrated in vacuo. The residue was re-crystallised from isobutanol (300 ml) at 65° C. and collected by filtration. The solid was washed with isobutanol (3×50 ml) and dried in a vacuum oven at 45° C. to give the title compound (61.2 g).

1H-NMR (d₆-DMSO): 2.09-2.11 (3H), 2.78-2.82 (2H), 2.82-2.85 (2H), 6.93-6.97 (1H), 7.00-7.05 (2H), 7.29-7.32 (1H), 7.46-7.50 (1H)

Similarly prepared were:

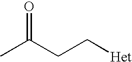

| | | From the |
|---|---|---|
| Preparation | Het | Compound of: |
| 44 | 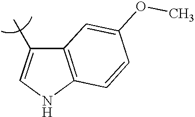 | Preparation 127 |
| 45 | 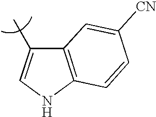 | Preparation 105 |
| 46 | 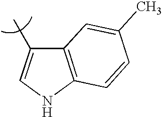 | Preparation 107 |
| 47 | 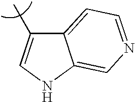 | Preparation 100 |
| 48 | 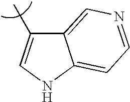 | Preparation 99 |
| 49 | 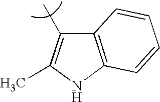 | Preparation 123 |
| 50 | 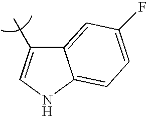 | Preparation 124 |
| 51 | 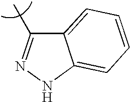 | Preparation 147 |

### Preparation 44

### 4-(5-Methoxy-1H-indol-3-yl)butan-2-one

¹H-NMR (CD₃OD): 2.11-2.13 (3H), 2.82-2.87 (2H), 2.93-2.98 (2H), 3.29-3.32 (3H), 6.72-6.76 (1H), 6.96-7.01 (2H), 7.17-7.21 (1H)

### Preparation 45

### 3-(3-Oxo-butyl)-1H-indole-5-carbonitrile

¹H-NMR (CD₃OD): 2.10-2.13 (3H), 2.80-2.86 (2H), 2.95-3.00 (2H), 7.12-7.13 (1H), 7.30-7.33 (1H), 7.40-7.43 (1H), 7.98-7.99 (1H)

### Preparation 46

### 4-(5-Methyl-1H-indol-3-yl)butan-2-one

¹H-NMR (CDCl₃): 2.13-2.16 (3H), 2.45-2.48 (3H), 2.81-2.87 (2H), 3.00-3.05 (2H), 6.93-6.96 (1H), 7.00-7.04 (1H), 7.22-7.27 (1H), 7.36-7.38 (1H)

### Preparation 47

### 4-(1H-Pyrrolo[2,3-c]pyridin-3-yl)butan-2-one

Experimental MH⁺ 189.1; expected 189.1

### Preparation 48

### 4-(1H-Pyrrolo[3,2-c]pyridin-3-yl)butan-2-one

Experimental MH⁺ 189.1; expected 189.1

### Preparation 49

### 4-(2-Methyl-1H-indol-3-yl)butan-2-one

Experimental MH⁺ 202.2; expected 202.1

### Preparation 50

### 4-(5-Fluoro-1H-indol-3-yl)butan-2-one

¹H-NMR (d₆-DMSO): 2.01-2.06 (3H), 2.70-1.84 (4H), 6.80-6.88 (1H), 7.10-7.12 (1H), 7.20-7.31 (2H)

### Preparation 51

### 4-(1H-Indazol-3-yl)butan-2-one

¹H-NMR (CDCl₃): 2.15-2.16 (3H), 3.18-3.20 (2H), 4.65-4.67 (2H), 7.02-7.04 (1H), 7.25-7.27 (1H), 7.61-7.65 (1H)

### Preparation 52

### 4-Methyl-4-(1-methyl-1H-indol-3-yl)pentan-2-one

To a mixture of the compound of Preparation 109 (1.0 g, 7.6 mmol) and the compound of Preparation 103 (748 mg, 7.6 mmol) in ethanol (20 ml) was added iodine (193 mg, 0.8 mmol) and the reaction mixture was stirred at room temperature for 18 h. The mixture was concentrated in vacuo and the residue was partitioned between ethyl acetate and water. The two layers were separated and the organic phase was washed with 10% aqueous sodium thiosulphate solution and brine, dried (MgSO₄) and concentrated in vacuo. The residue was purified by automated flash chromatography (Biotage™ 25M cartridge) with gradient elution, ethyl acetate:cyclohexane [20:80 to 80:20]. The appropriate fractions were combined and concentrated to give the title compound (470 mg).

¹H-NMR (CDCl₃): 1.52-1.56 (6H), 1.73-1.75 (3H), 2.93-2.96 (2H), 3.73-3.75 (3H), 6.79-6.81 (1H), 7.08-7.14 (1H), 7.19-7.26 (1H), 7.29-7.32 (1H), 7.78-7.81 (1H)

Similarly prepared was:

### Preparation 53

### 4-(1H-Indol-3-yl)-4-methylpentan-2-one

From the compound of Preparation 121.

¹H-NMR (CDCl₃): 1.53-1.56 (6H), 1.72-1.74 (3H), 2.94-2.97 (2H), 6.93-6.95 (1H), 7.10-7.22 (2H), 7.36-7.40 (1H), 7.79-7.83 (1H)

### Preparation 54

### 4-(5-Chloro-1H-indol-7-yl)but-3-en-2-one

To a solution of the compound of Preparation 37 (665 mg, 3.7 mmol) in tetrahydrofuran (10 ml) was added the compound of Preparation 93 (2.4 g, 7.4 mmol) and the reaction mixture was heated at reflux for 18 h. The mixture was concentrated in vacuo and the residue was partitioned between diethyl ether and water. The two layers were separated and the organic phase was washed with water and brine, dried (MgSO₄) and concentrated in vacuo. The residue was dissolved in dichloromethane (6 ml) purified by automated flash chromatography (Biotage™ 25M cartridge) with gradient elution, ethyl acetate: cyclohexane [2:98 to 50:50]. The appropriate fractions were combined and concentrated to give the title compound (680 mg).

¹H-NMR (CDCl₃): 2.43-2.45 (3H), 6.55-6.58 (1H), 6.84-6.89 (1H), 7.29-7.32 (1H), 7.40-7.43 (1H), 7.65-7.68 (1H), 7.86-7.93 (1H)

### Preparation 55

### 4-(1,3-Thiazol-5-yl)but-3-en-2-one

To a solution of the compound of Preparation 96 (1.1 g, 9.5 mmol) in tetrahydrofuran (30 ml) was added the compound of Preparation 93 (6.1 g, 19.1 mmol) and the reaction mixture was heated at reflux for 4 h. The mixture was concentrated in vacuo and the residue was partitioned between water (25 ml) and dichloromethane (25 ml). The two layers were separated and the aqueous phase was extracted with dichloromethane (2×25 ml). The combined organic phases were washed with brine, dried (MgSO₄) and concentrated in vacuo. The residue was purified by automated flash chromatography (Biotage™, 65i silica cartridge) with gradient elution, ethyl acetate:cyclohexane [20:80 to 60:40]. The appropriate fractions were combined and concentrated to give the title compound (1.2 g).

¹H-NMR (CD₃OD): 2.34-2.36 (3H), 6.56-6.63 (1H), 7.83-7.89 (1H), 8.14-8.18 (1H), 9.04-9.07 (1H)

Similarly prepared was:

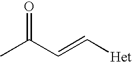

| | | From the |
|---|---|---|
| Preparation | Het | Compound of: |
| 56 | 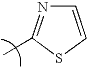 | Preparation 110 |
| 57 | 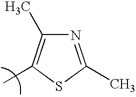 | Preparation 114 |
| 58 | 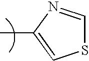 | Preparation 117 |
| 59 | 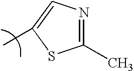 | Preparation 95 |
| 60 | 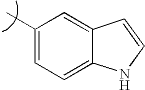 | Preparation 135 |

### Preparation 56

### 4-(1,3-Thiazol-2-yl)but-3-en-2-one

¹H-NMR (CDCl₃): 2.38-2.41 (3H), 6.91-6.97 (1H), 7.44-7.47 (1H), 7.60-7.67 (1H), 7.92-7.95 (1H)

### Preparation 57

### 4-(2,4-Dimethyl-1,3-thiazol-5-yl)but-3-en-2-one

¹H-NMR (CDCl₃): 2.31-2.34 (3H), 2.47-2.50 (3H), 2.66-2.69 (3H), 6.28-6.34 (1H), 7.57-7.62 (1H)

### Preparation 58

### 4-Thiazol-5-yl-but-3-en-2-one

¹H-NMR (CDCl₃): 2.36-2.38 (3H), 7.04-7.10 (1H), 7.50-7.55 (2H), 8.83-8.86 (1H)

### Preparation 59

### 4-(2-Methyl-thiazol-5-yl)-but-3-en-2-one

¹H-NMR (CD₃OD): 2.31-2.35 (3H), 2.70-2.73 (3H), 6.42-6.49 (1H), 7.74-7.89 (2H)

### Preparation 60

### 4-(1H-Indol-5-yl)but-3-en-2-one

Experimental (M-H⁺)⁻ 184.0; expected 184.1

### Preparation 61

### 4-(4-Methyl-1,3-thiazol-5-yl)but-3-en-2-one

To a solution of sodium hydride (60% dispersion in oil, 639 mg, 16.0 mmol) in tetrahydrofuran (5 ml) was added dropwise the compound of Preparation 115 (2.9 g, 14.8 mmol) in tetrahydrofuran (10 ml). After stirring for 1.5 h, the solution was cooled to 0° C. and the compound of Preparation 118 (1.5 g, 11.4 mmol) in tetrahydrofuran (10 ml) was added dropwise. The reaction mixture was stirred at room temperature for 18 h and then diluted with dichloromethane (20 ml). The solution was washed with water (20 ml) and the aqueous washings were extracted with dichloromethane (2×20 ml). The combined organic phases were washed with brine, dried (MgSO₄) and concentrated in vacuo. The residue was purified by automated flash chromatography (Biotage 40+M cartridge) with gradient elution, ethyl acetate:cyclohexane [12:88 to 100:0]. The appropriate fractions were combined and concentrated to give the title compound (1.5 g).

¹H-NMR (CDCl₃): 2.30-2.32(3H), 2.55-2.57 (3H), 6.39-6.43 (1H), 7.60-7.64 (1H), 8.62-8.64 (1H)

Similarly prepared was:

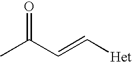

| | | From the |
|---|---|---|
| Preparation | Het | Compound of: |
| 62 | 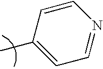 | Preparation 128 |
| 63 | 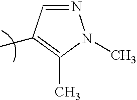 | Preparation 112 |
| 64 | 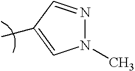 | Preparation 146 |
| 65 | 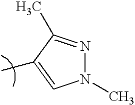 | Preparation 90 |
| 66 | 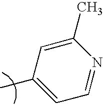 | Preparation 150 |

### Preparation 62

### 4-Pyridin-4-ylbut-3-en-2-one

¹H-NMR (CDCl₃): 2.39-2.42(3H), 6.80-6.86 (1H), 7.39-6.45 (3H), 8.70-8.73 (2H)

### Preparation 63

### 4-(1,5-Dimethyl-1H-pyrazol-4-yl)but-3-en-2-one

The title compound was used directly.

### Preparation 64

### 4-(1-Methyl-1H-pyrazol-4-yl)but-3-en-2-one

¹H-NMR (CDCl₃): 2.30-2.31 (3H), 3.91-3.92 (3H), 6.42-6.44 (1H), 7.39-7.41 (1H), 7.57-7.58 (1H), 7.70-7.71 (1H)

### Preparation 65

### 4-(1,3-Dimethyl-1H-pyrazol-4-yl)but-3-en-2-one

The title compound was used directly.

### Preparation 66

### 4-(2-Methylpyridin-4-yl)but-3-en-2-one

The title compound was used directly.

### Preparation 67

### 6-(3-Oxobutyl)pyridin-2(1H)-one

To a solution of the compound of Preparation 12 (400 mg, 2.2 mmol) in dichloromethane (5 ml) was added the compound of Preparation 132 (0.6 ml, 4.5 mmol) and the reaction mixture was heated at reflux for 2 h. The mixture was concentrated in vacuo and to the residue was added 20% methanol:dichloromethane. The solution was filtered to remove any solid material and the filtrate was concentrated in vacuo to give the title compound (50 mg).

¹H-NMR (d₆-DMSO): 2.07-2.09 (3H), 3.77-3.83 (4H), 7.29-7.36 (3H), 7.54-7.59 (1H)

### Preparation 68

### 6-Methoxypyridine-2-carbaldehyde

To a solution of the compound of Preparation 113 (5.6 g, 29.8 mmol) in anhydrous tetrahydrofuran (100 ml), at −78° C. and under nitrogen, was added n-butyllithium (1.6M in hexane, 19.5 ml), via syringe. The mixture was stirred at −78° C. for 30 min, before addition of N,N-dimethylformamide (2.5 ml, 32.8 mmol). The reaction mixture was allowed to warm to room temperature and stirred for 18 h, before being acidified with sulphuric acid (2M) and then neutralised by addition of sodium hydrogen carbonate. The mixture was concentrated in vacuo and the residue was extracted with ethyl acetate (4×150 ml). The combined extracts were dried (MgSO₄) and concentrated in vacuo to give the title compound (3.0 g).

¹H-NMR (CDCl₃): 4.01-4.05 (3H), 6.95-7.00 (1H), 7.54-7.58 (1H), 7.70-7.76 (1H), 9.95-9.98 (1H)

### Preparation 69

### 4-(1H-Benzimidazol-2-yl)butan-2-one

A mixture of the compound of Preparation 102 (10.0 g, 92.5 mmol) and the compound of Preparation 101 (9.9 ml, 92.5 mmol) in hydrochoric acid (6N, 100 ml) was heated at reflux for 18 h. Charcoal (5 g) was added and the mixture was stirred for 30 min. The mixture was filtered through Arbocel® and the filtrate was adjusted to pH 9 by addition of ammonia solution. The resulting mixture was extracted with ethyl acetate and the combined organic extracts were washed with water and brine, dried (MgSO₄) and concentrated in vacuo. The residue was re-crystallised from ethyl acetate:diethyl ether and the solid was washed with diethyl ether to give the title compound (4.5 g).

¹H-NMR (CDCl₃): 2.17-2.22 (3H), 3.01-3.08 (2H), 3.12-3.19 (2H), 7.17-7.24 (2H), 7.48-7.56 (2H)

### Preparation 70

### 4-(1,3,5-Trimethyl-1H-pyrazol-4-yl)butan-2-one

A mixture of the compound of Preparation 133 (48.0 g, 203.0 mmol), palladium (II) acetate (2.3 g, 10.2 mmol), the compound of Preparation 134 (53.9 ml, 610.0 mmol), N,N-diisopropylethylamine (142.0 ml, 813.0 mmol) and lithium chloride (25.9 g, 610.0 mmol) in N,N-dimethylformamide (480 ml) was heated at 120° C., under nitrogen, for 24 h. The mixture was cooled and concentrated in vacuo and to the residue was added water (250 ml). The solution was extracted with ethyl acetate (3×200 ml) and the combined organic extracts were washed with brine (250 ml), dried (MgSO₄) and concentrated in vacuo. The residue was purified by column chromatography (silica, 200 g), eluting with ethyl acetate. The appropriate fractions were combined and concentrated and to the residue was added cyclohexane (250 ml). The slurry was stirred for 2 h, keeping the temperature below 10° C., and then filtered. The residue was re-dissolved in tert-butyl methyl ether and concentrated in vacuo to give the title compound (22.0 g).

Experimental MH⁺ 181.2; expected 181.1

### Preparation 71

### 4-[2-(2,5-Dimethyl-1H-pyrrol-1-yl)pyridin-3-yl]butan-2-one

A mixture of the compound of Preparation 74 (237 mg, 0.9 mmol), the compound of Preparation 134 (0.3 ml, 3.3 mmol), triethylamine (0.5 ml, 3.3 mmol), palladium (II) acetate (21 mg) and lithium chloride (40 mg, 0.9 mmol) in N,N-dimethylformamide (10 ml) was de-gassed and heated at 150° C. in a microwave oven (CEM 300 W) for 20 min. To the reaction mixture was added diethyl ether (50 ml) and the solution was washed with water (50 ml and 2×20 ml). The organic phase was dried (K₂CO₃) and concentrated in vacuo to give the title compound (200 mg).

¹H-NMR (CDCl₃): 1.96-1.98 (6H), 2.06-2.07 (3H), 2.45-2.47 (2H), 2.62-2.68 (2H), 5.89-5.91 (2H), 7.29-7.31 (1H), 7.70-7.72 (1H), 8.47-8.49 (1H)

Similarly prepared were:

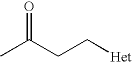

| | | From the |
|---|---|---|
| Preparation | Het | Compound of: |
| 72 | 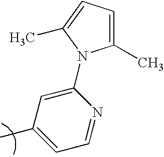 | Preparation 75 |
| 73 | 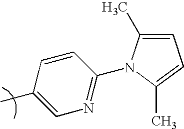 | Preparation 76 |

### Preparation 72

### 4-[5-(2,5-Dimethyl-1H-pyrrol-1-yl)pyridin-3-yl]butan-2-one

¹H-NMR (CDCl₃): 2.01-2.03 (6H), 2.17-2.19 (3H), 2.80-2.83 (2H), 2.96-2.99 (2H), 5.92-5.94 (2H), 7.40-7.41 (1H), 8.36-8.37 (1H), 8.48-8.49 (1H)

### Preparation 73

### 4-[6-(2,5-Dimethyl-1H-pyrrol-1-yl)pyridin-3-yl]butan-2-one

¹H-NMR (CDCl₃): 2.10-2.12 (6H), 2.19-2.20 (3H), 2.81-2.84 (2H), 2.94-2.97 (2H), 5.93-5.95 (2H), 7.14-7.16 (1H), 7.62-7.64 (1H), 8.41-8.42 (1H)

### Preparation 74

### 3-Bromo-2-(2,5-dimethyl-1H-pyrrol-1-yl)pyridine

A mixture of the compound of Preparation 137 (2.0 g, 11.6 mmol), the compound of Preparation 136 (1.6 g, 13.9 mmol) and p-toluenesulphonic acid monohydrate (22 mg, 0.1 mmol) in toluene (15 ml) was heated at reflux in a Dean Stark apparatus for 18 h. To the mixture was added ethyl acetate (60 ml) and the solution was washed with aqueous sodium hydrogen carbonate solution (30 ml) and water (2×10 ml), dried (K₂CO₃) and concentrated in vacuo. The residue was dissolved in diethyl ether and passed through a silica plug, eluting with diethyl ether. The filtrate was concentrated in vacuo to give the title compound (2.3 g).

¹H-NMR (CDCl₃): 1.99-2.01 (6H), 5.89-5.91 (2H), 7.24-7.27 (1H), 8.03-8.05 (1H), 8.57-8.59 (1H)

Similarly prepared were:

### Preparation 75

### 3-Bromo-5-(2,5-dimethyl-1H-pyrrol-1-yl)pyridine

From the compound of Preparation 138.

¹H-NMR (CDCl₃): 2.02-2.05 (6H), 5.93-5.95 (2H), 7.75-7.77 (1H), 8.43-8.45 (1H), 8.71-8.73 (1H)

### Preparation 76

### 5-Bromo-2-(2,5-dimethyl-1H-pyrrol-1-yl)pyridine

From the compound of Preparation 139.

¹H-NMR (CDCl₃): 2.12-2.14 (6H), 5.90-5.92 (2H), 7.10-7.1 (1H), 7.92-7.95 (1H), 8.62-8.64 (1H)

### Preparation 77

### (6R,7R)-6-({3-[2-(2,5-Dimethyl-1H-pyrrol-1-yl)pyridin-3-yl]-1-methylpropyl}amino)-7-hydroxy-4,5,6,7-tetrahydroimidazo[4,5,1-jk][1]benzazepin-2(1H)-one

To a mixture of the compound of Preparation 9 (300 mg, 1.2 mmol) and the compound of Preparation 71 (341 mg, 1.4 mmol) in methanol (10 ml), under nitrogen, was added triethylamine (49 ml, 0.4 mmol). After stirring for 20 min, sodium cyanoborohydride (111 mg, 1.8 mmol) was added and the reaction mixture was heated at 60° C., under nitrogen, for 18 h. After cooling, citric acid (500 mg) was added and the mixture was heated at 60° C. for 3 h. To the mixture was added water (0.2 ml), followed by excess sodium hydrogen carbonate and the mixture was stirred at room temperature for 18 h. The mixture was pre-absorbed onto silica (10 g) and passed through a silica plug (10 g), eluting with dichloromethane:2.5% methanolic ammonia [4:1]. The filtrate was concentrated in vacuo to give the title compound (500 mg) as a mixture of non-racemic diastereoisomers.

HPLC method A—retention times 13.45 and 13.89 min

Similarly prepared were:

### Preparation 78

### (6R,7R)-6-({3-[5-(2,5-Dimethyl-1H-pyrrol-1-yl)pyridin-3-yl]-1-methylpropyl}amino)-7-hydroxy-4,5,6,7-tetrahydroimidazo[4,5,1-jk][1]benzazepin-2(1H)-one

From the compound of Preparation 72, as a mixture of non-racemic diastereoisomers.

HPLC method A—retention times 14.14 and 14.46 min

### Preparation 79

### (6R,7R)-6-({3-[6-(2,5-Dimethyl-1H-pyrrol-1-yl)pyridin-3-yl]-1-methylpropyl}amino)-7-hydroxy-4,5,6,7-tetrahydroimidazo[4,5,1-jk][1]benzazepin-2(1H)-one

From the compound of Preparation 73, as a mixture of non-racemic diastereoisomers.

HPLC method A—retention times 14.62 and 14.88 min

### Preparation 80

### Isothiazole-4-carbaldehyde

To a solution of the compound of Preparation 140 (3.9 g, 9.1 mmol) in dichloromethane (31 ml) was added the compound of Preparation 82 (950 mg, 8.3 mmol). The reaction mixture was stirred at room temperature, under nitrogen, for 18 h and then filtered through Celite®, washing through with diethyl ether. The filtrate was concentrated in vacuo to give the title compound (1.2 g).

¹H-NMR (CD₃OD): 8.46-8.47 (1H), 8.80-8.81 (1H)

Similarly prepared was:

### Preparation 81

### 3-Bromoisoxazole-5-carbaldehyde

From the compound of Preparation 83.

The title compound was used directly.

### Preparation 82

### Isothiazol-4-ylmethanol

To a solution of the compound of Preparation 141 (1.4 g, 11.0 mmol) in tetrahydrofuran (6 ml), at −5° C., was added dropwise borane (1M in tetrahydrofuran, 16.5 ml). The reaction mixture was allowed to warm to room temperature and stirred for 18 h. The mixture was quenched by addition of water: acetic acid (1:1, 4 ml) and the mixture was concentrated in vacuo. The residue was added to saturated aqueous sodium hydrogen carbonate solution (5.5 ml) at 0° C. and the two layers were separated. The aqueous layer was extracted with ethyl acetate (750 ml) and the combined extracts were concentrated in vacuo to give the title compound (700 mg).

¹H-NMR (CDCl₃): 4.80-4.84 (2H), 8.69-8.71 (1H), 8.76-8.78 (1H)

Similarly prepared was

### Preparation 83

### (3-Bromoisoxazol-5-yl)methanol

From the compound of Preparation 143.

The title compound was used directly.

### Preparation 84

### 4-(1H-Benzimidazol-1-yl)butan-2-one

To a suspension of the compound of Preparation 142 (1.0 g, 8.5 mmol) and the compound of Preparation 94 (0.8 ml, 9.3 mmol) in dichloromethane (20 ml) was added zirconium (IV) chloride (100 mg, 0.4 mmol), The reaction mixture was stirred at room temperature for 16 h and then partitioned between dichloromethane and water. The organic phase was separated, dried (MgSO₄) and concentrated in vacuo. The residue was purified by column chromatography (silica) with gradient elution, dichloromethane 2% methanolic ammonia [99:1 to 95:5]. The appropriate fractions were combined and concentrated to give the title compound (1.0 g).

¹H-NMR (CDCl₃): 2.09-2.11 (3H), 2.97-3.00 (2H), 4.44-4.47 (2H), 7.26-7.32 (2H), 7.38-7.40 (1H), 7.78-7.80 (1H), 7.97-7.98 (1H)

Similarly prepared were:

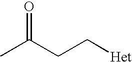

| | | From the |
|---|---|---|
| Preparation | Het | Compound of: |
| 85 | 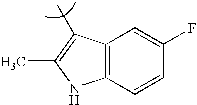 | Preparation 144 |
| 86 | 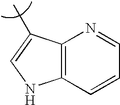 | Preparation 145 |
| 87 | 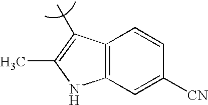 | Preparation 148 |
| 88 | 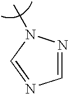 | Preparation 149 |
| 89 | 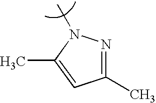 | Preparation 151 |

### Preparation 85

### 4-(5-Fluoro-2-methyl-1H-indol-3-yl)butan-2-one

¹H-NMR (CDCl₃): 2.08-2.10 (3H), 2.37-2.39 (3H), 2.70-2.73 (2H), 2.88-2.91 (2H), 6.80-6.84 (1H), 7.09-7.11 (1H), 7.12-7.15 (1H)

### Preparation 86

### 4-(1H-Pyrrolo[3,2-b]pyridin-3-yl)butan-2-one

No n.m.r. data available.

### Preparation 87

### 3-(3-Oxobutyl)-1H-indole-6-carbonitrile

¹H-NMR (d₆-DMSO): 2.08-2.09 (3H), 2.78-2.80 (2H), 2.83-2.85 (2H), 7.25-7.27 (1H), 7.39-7.40 (1H), 7.65-7.68 (2H)

### Preparation 88

### 4-(1H-1,2,4-Triazol-1-yl)butan-2-one

¹H-NMR (CDCl₃): 2.17-2.19 (3H), 3.02-3.05 (2H), 4.40-4.43 (2H), 7.88-7.89 (1H), 8.12-8.13 (1H)

### Preparation 89

### 4-(3,5-Dimethyl-1H-pyrazol-1-yl)butan-2-one

Experimental MH⁺ 167.1; expected 167.1

### Preparation 90-151

The following compounds were obtained commercially:

| Preparation | Compound |
|---|---|
| 90 | 1,3-Dimethyl-1H-pyrazole-4-carbaldehyde |
| 91 | 1-Butanol |
| 92 | 4-Pyrazol-1-yl-butan-2-one |
| 93 | 1-Triphenylphosphoranylidene-2-propanone |
| 94 | Methyl vinyl ketone |
| 95 | 2-Methyl-1,3-thiazole-5-carbaldehyde |
| 96 | 1,3-Thiazole-5-carbaldehyde |
| 97 | 1-Furan-2-yl-propan-2-one |
| 98 | 1H-Indole-7-carbaldehyde |
| 99 | 1H-Pyrrolo[3,2-c]pyridine |
| 100 | 1H-Pyrrolo[2,3-c]pyridine |
| 101 | 4-Oxo-pentanoic acid |
| 102 | Benzene-1,2-diamine |
| 103 | 4-Methyl-pent-3-en-2-one |
| 104 | 5-Chloro-2-nitro-benzaldehyde |
| 105 | 1H-Indole-5-carbonitrile |
| 106 | Di-tert-butyl dicarbonate |
| 107 | 5-Methyl-1H-indole |
| 108 | 5-Chloro-1H-indole |
| 109 | 1-Methyl-1H-indole |
| 110 | Thiazole-2-carbaldehyde |
| 111 | 5-Fluoro-2-nitro-benzaldehyde |
| 112 | 1,5-Dimethyl-1H-pyrazole-4-carbaldehyde |
| 113 | 2-Bromo-6-methoxy-pyridine |
| 114 | 2,4-Dimethyl-thiazole-5-carbaldehyde |
| 115 | (2-Oxo-propyl)-phosphonic acid diethyl ester |
| 116 | 4-(2-Chloro-thiazol-5-yl)-butan-2-one |
| 117 | Thiazole-4-carbaldehyde |
| 118 | 4-Methyl-thiazole-5-carbaldehyde |
| 119 | 3-Hydroxy-pyridine-2-carbaldehyde |
| 120 | Benzofuran-5-carbaldehyde |
| 121 | 1H-Indole |
| 122 | 3-(2-Bromo-ethyl)-1H-indole |
| 123 | 2-Methyl-1H-indole |
| 124 | 5-Fluoro-1H-indole |
| 125 | Ethyl acetoacetate |
| 126 | 1-(5-Methylamino-[1,2,4]thiadiazol- |
| | 3-yl)-propan-2-one |
| 127 | 5-Methoxy-1H-indole |
| 128 | Pyridine-4-carbaldehyde |
| 129 | 2-Oxo-1,2-dihydro-pyridine-3-carbaldehyde |
| 130 | Ethyl, 4-bromobutyrate |
| 131 | Vinyl magnesium bromide |
| 132 | Trimethyl silyl iodide |
| 133 | 4-Iodo-1,3,5-trimethyl-1H-pyrazole |
| 134 | But-3-en-2-ol |
| 135 | 1H-Indole-5-carbaldehyde |
| 136 | Hexane-2,5-dione |
| 137 | 3-Bromopyridin-2-amine |
| 138 | 5-Bromopyridin-3-amine |
| 139 | 5-Bromopyridin-2-amine |
| 140 | Dess-Martin periodinane |
| 141 | Isothiazole-4-carboxylic acid |
| 142 | 1H-Benzimidazole |
| 143 | 3-Bromoisoxazole-5-carboxylic acid |
| 144 | 5-Fluoro-2-methyl-1H-indole |
| 145 | 1H-Pyrrolo[3,2-b]pyridine |
| 146 | 1-Methyl-1H-pyrazole-4-carbaldehyde |
| 147 | 1H-Indazole |
| 148 | 1H-Indole-6-carbonitrile |
| 149 | 1H-1,2,4-Triazole |
| 150 | 2-Methylisonicotinaldehyde |
| 151 | 3,5-Dimethyl-1H-pyrazole |
| 152 | Chlorotris(triphenylphosphine) rhodium(I) |

Compounds may be obtained from the following commercial suppliers:

Sigma-Aldrich, P O Box 14508, St. Louis, Mo., 63178, USA Lancaster Synthesis Ltd., Newgate, White Lund, Morecambe, Lancashire, LA3 3BN, UK

Maybridge, Trevillett, Tintagel, Cornwall, PL34 0HW, UK

Fluorochem Ltd., Wesley Street, Old Glossop, Derbyshire, SK13 7RY, UK

ASDI Inc, 601 Interchange Blvd., Newark, Del., 19711, USA

Alfa Aesar, 26 Parkridge Road, Ward Hill, Mass., 01835, USA

Bionet Research Ltd., Highfield Industrial Estate, Camelford, Cornwall, PL32 9QZ, UK

Fulcrum Scientific Ltd.,P O Box 1489, Huddersfield, West Yorkshire, HD1 9FG, UK

MicroChemistry Ltd., Kosygin St. 4, Moscow 119993, Russia

### Preparation 153

### 3-(1H-Indol-3-yl)propanal

WO 2005051878 A1

### Preparation 154

### 4-(1-Methyl-1H-indol-3-yl)-butan-2-one

Tetrahedron (2005), 61(40), 9541-9544

### Preparation 155

### 1H-Benzimidazole-5-carbaldehyde

Journal of Heterocyclic Chemistry (1976), 13(5), 1121-3.

### Preparation 156

Methyl 5-(3-oxobutyl)-2-furoate

Chemistry Letters (1978), (5), 529-532.

### Preparation 157

### 4-(1-Benzyl-1H-indol-3-yl)-butan-2-one

U.S. Pat. No. 3,671,544 Example 1

### Preparation 158

### 4-(5-Benzyloxy-1H-indol-3-yl)-butan-2-one

Journal of Organic Chemistry (2003), 68(6), 2109-2114.

### Preparation 159

### 3-(2-Oxo-propyl)-1H-indole-2-carboxylic acid ethyl ester

Journal of Heterocyclic Chemistry (1981), 18(5), 889-92.

### Preparation 160

### 4-Pyridin-2-yl-butan-2-one

Bulletin de la Societe Chimique de France (1960), No. 2, 322-5

### Preparation 161

### 4-Imidazol-1-yl-butan-2-one U.S. Pat. No. 3,949,076 Example 3.

### Preparation 162

### 6-Chloro-3-(2-chloro-ethyl)-1H-indole-2-carboxylic acid ethyl ester

EP396124 Example 47.

1. A compound of formula (I) 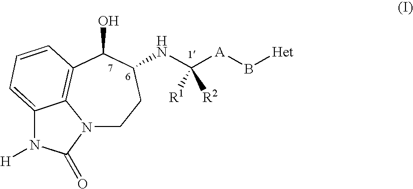
or a pharmaceutically acceptable prodrug thereof, or a pharmaceutically or veterinarily acceptable salt of said compound or prodrug, wherein:
A is CH₂, CH(C₁-C₃ alkyl) or C(C₁-C₃ alkyl)₂; and
B is a covalent bond, —CR^{A}R^{B}—, —CR^{A}R^{B}—CR^{C}R^{D}, CR^{A}R^{B}—CR^{C}R^{D}—CR^{E}R^{F}—, —CR^{A}R^{B}—O—, —O—CR^{A}R^{B}, —O—CR^{A}R^{B}—CR^{C}R^{D}, —CR^{A}R^{B}—O—CR^{C}R^{D}, or —CR^{A}R^{B}—CR^{C}R^{D}—O—;
or -A-B— is —CR^{A}═CR^{B}—;
R^{A}, R^{B}, R^{C}, R^{D}, R^{E} and R^{F} are each independently H or C₁-C₃ alkyl;
R¹ and R² are each independently H or C₁-C₃ alkyl, or R¹ and R² together with the carbon atom to which they are attached form a 3- to 6-membered saturated carbocyclic ring; and
Het is a 5- or 6-membered monocyclic or 9- or 10-membered bicyclic heteroaryl group which may optionally be substituted with up to 3 groups independently selected from halo, —CN, C₁-C₄ alkyl, —CH₂Ph, —OH, —O—(C₁-C₄ alkyl), —O—CH₂—(C₃-C₆)cycloalkyl, —O—CH₂Ph, —NH₂, —NH(C₁-C₄ alkyl), —N(C₁-C₄ alkyl)₂, —CONH₂, —CONH(C₁-C₄ alkyl), —CON(C₁-C₄ alkyl)₂, —CO₂H and —CO₂(C₁-C₄ alkyl).

2. A compound according to claim 1, or a pharmaceutically acceptable prodrug thereof, or a pharmaceutically or veterinarily acceptable salt of said compound or prodrug, wherein R^{A}, R^{B}, R^{C}, R^{D}, R^{E} and R^{F} are each independently H or methyl.

3. A compound according to claim 2, or a pharmaceutically acceptable prodrug thereof, or a pharmaceutically or veterinarily acceptable salt of said compound or prodrug, wherein A is CH₂ and B is a covalent bond, CH₂ or C(CH₃)₂, or -A-B— is —CH═CH—.

4. A compound according to claim 3, or a pharmaceutically acceptable prodrug thereof, or a pharmaceutically or veterinarily acceptable salt of said compound or prodrug, wherein A is CH₂ and B is CH₂.

5. A compound according to claim 1, or a pharmaceutically acceptable prodrug thereof, or a pharmaceutically or veterinarily acceptable salt of said compound or prodrug, wherein R¹ and R² are each independently H or CH₃.

6. A compound according to claim 5, or a pharmaceutically acceptable prodrug thereof, or a pharmaceutically or veterinarily acceptable salt of said compound or prodrug, wherein one of R¹ and R² is CH₃ and the other is H.

7. A compound according to claim 6, or a pharmaceutically acceptable prodrug thereof, or a pharmaceutically or veterinarily acceptable salt of said compound or prodrug, wherein R¹ is H and R² is CH₃.

8. A compound according to claim 7, or a pharmaceutically acceptable prodrug thereof, or a pharmaceutically or veterinarily acceptable salt of said compound or prodrug, wherein the absolute stereochemistry at C-1′, C-6 and C-7 is R, R, R.

9. A compound according to claim 1, or a pharmaceutically acceptable prodrug thereof, or a pharmaceutically or veterinarily acceptable salt of said compound or prodrug, wherein Het is selected from pyrazolyl, imidazolyl, thiazolyl, isothiazolyl, pyridyl, indolyl and pyrrolopyridinyl, each of which may optionally be substituted with up to 3 groups independently selected from halo, —CN, (C₁-C₄)alkyl, —OH, —O—(C₁-C₄ alkyl), —NH(C₁-C₄ alkyl), —CO₂H, —CO₂(C₁-C₄ alkyl), —CH₂Ph, —O—CH₂Ph and —NH₂.

10. A compound according to claim 1 selected from:
(6R*,7R*)-7-hydroxy-6-{[(1R*)-1-methyl-3-(1,3-thiazol-5-yl)propyl]amino}-4,5,6,7-tetrahydroimidazo[4,5,1-jk][1]benzazepin-2(1H)-one;
(6R*,7R*)-7-hydroxy-6-{[(1S*)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1RS)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1R)-1-methyl-3-(1,3-thiazol-5-yl)propyl]amino}-4,5,6,7-tetrahydroimidazo[4,5,1-jk][1]benzazepin-2(1H)-one;
(6R,7R)-7-hydroxy-6-{[(1S)-1-methyl-3-(1,3-thiazol-5-yl)propyl]amino}-4,5,6,7-tetrahydroimidazo[4,5,1-jk][1]benzazepin-2(1H)-one;
(6R*,7R*)-7-hydroxy-6-{[(1R*)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R*,7R*)-7-hydroxy-6-{[(1S*)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1RS)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1R)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R,7R)-7-hydroxy-6-{[(1S)-1-methyl-3-(1,3,5-trimethyl-1H-pyrazol-4-yl)propyl]amino}-4,5,6,7-tetrahydroimidazo[4,5,1-jk][1]benzazepin-2(1H)-one;
(6R*,7R*)-7-hydroxy-6-{[(1R*)-3-isothiazol-4-yl-1-methylpropyl]amino}-4,5,6,7-tetrahydroimidazo[4,5,1-jk][1]benzazepin-2(1H)-one;
(6R*,7R*)-7-hydroxy-6-{[(1S*)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1RS)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1R)-3-isothiazol-4-yl-1-methylpropyl]amino}-4,5,6,7-tetrahydroimidazo[4,5,1-jk][1]benzazepin-2(1H)-one;
(6R,7R)-7-hydroxy-6-{[(1S)-3-isothiazol-4-yl-1-methylpropyl]amino}-4,5,6,7-tetrahydroimidazo[4,5,1-jk][1]benzazepin-2(1H)-one;
(6R*,7R*)-6-{[(1R*)-3-(2-aminopyridin-3-yl)-1-methylpropyl]amino}-7-hydroxy-4,5,6,7-tetrahydroimidazo[4,5,1-jk][1]benzazepin-2(1H)-one;
(6R*,7R*)-6-{[(1S*)-3-(2-aminopyridin-3-yl)-1-methylpropyl]amino}-7-hydroxy-4,5,6,7-tetrahydroimidazo[4,5,1-jk][1]benzazepin-2(1H)-one;
(6R,7R)-6-{[(1RS)-3-(2-aminopyridin-3-yl)-1-methylpropyl]amino}-7-hydroxy-4,5,6,7-tetrahydroimidazo[4,5,1-jk][1]benzazepin-2(1H)-one;
(6R,7R)-6-{[(1R)-3-(2-aminopyridin-3-yl)-1-methylpropyl]amino}-7-hydroxy-4,5,6,7-tetrahydroimidazo[4,5,1-jk][1]benzazepin-2(1H)-one; and
(6R,7R)-6-{[(1S)-3-(2-aminopyridin-3-yl)-1-methylpropyl]amino}-7-hydroxy-4,5,6,7-tetrahydroimidazo[4,5,1-jk][1]benzazepin-2(1H)-one;
or a pharmaceutically acceptable prodrug thereof, or a pharmaceutically or veterinarily acceptable salt of said compound or prodrug.

11. A feed additive for a livestock animal comprising a compound according to claim 1, or a pharmaceutically acceptable prodrug thereof, or a pharmaceutically or veterinarily acceptable salt of said compound or prodrug.

12. A method of improving meat yield or meat quality in a livestock animal, comprising administering to said livestock animal an effective amount of a compound according to claim 1, or a pharmaceutically acceptable prodrug thereof, or a pharmaceutically or veterinarily acceptable salt of said compound or prodrug.

13. The method of claim 12 wherein the compound is administered in the animal feed.

14. The method of claim 12 wherein the compound is administered in combination with one or more other agents selected from steroids, bovine or porcine somatotropin, antibiotics, polyether ionophores, anticoccidials, other anabolic agents, antiparasitic agents, sodium bicarbonate, acarbose or other amylase or glycosidase inhibitors, enzymes, amino-acids, minerals and other supplements.

15. The method of claim 12 wherein the livestock animal is bovine or porcine.

16. The method of claim 12 wherein the livestock animal is an avian.

17. A compound according to claim 1, or a pharmaceutically acceptable prodrug thereof, or a pharmaceutically or veterinarily acceptable salt of said compound or prodrug, for use as a medicament.

18. A pharmaceutical composition comprising a compound according to claim 1, or a pharmaceutically acceptable prodrug thereof, or a pharmaceutically or veterinarily acceptable salt of said compound or prodrug, and a pharmaceutically acceptable carrier.
